(19) United States
(12) Patent Application Publication
(10) Pub. No.: US 2026/0275401 A1
(43) Pub. Date: Sep. 17, 2026

(54) MICROBIOME AS A BIOMARKER FOR PROSTATE CANCER

(71) Applicants: The Board of Regents of the University of Texas System, Austin, TX (US); The United States Government as represented by the Department of Veterans Affairs, Washington, DC (US)

(72) Inventor: Michael A. LISS, San Antonio, TX (US)

(21) Appl. No.: 19/471,017

(22) PCT Filed: Mar. 29, 2024
(86) PCT No.: PCT/US2024/022324

§ 371 Date: Sep. 30, 2025

Related U.S. Application Data
(60) Provisional application No. 63/456,086, filed on Mar. 31, 2023.
(60) Provisional application No. 63/546,591, filed on Oct. 31, 2023.

(51) Int. Cl. C12Q 1/68; C12Q 1/06; G16B 25/10; G16H 10/40; G16H 10/60; G16H 50/30

(52) U.S. Cl. CPC C12Q 1/68; C12Q 1/06; G16B 25/10; G16H 10/40; G16H 10/60; G16H 50/30; C12Q 2600/112

(57) ABSTRACT

Systems and methods are provided for a gut microbiome signature for cancers, including prostate cancer (PCa), using a prostate specific antigen (PSA)-independent, gut microbiome-based PCa biomarker, which can be used for diagnosis, suitability' for screening, treatment decisions and as part of a treatment method.

### CROSS-REFERENCE TO RELATED APPLICATIONS

This application claims benefit of U.S. Provisional Application No. 63/456,086, filed Mar. 31, 2023 and of U.S. Provisional Application No. 63/546,591, filed Oct. 31, 2023, the contents of each of which are hereby incorporated by reference.

### FIELD OF INVENTION

The present invention generally relates to systems and method for using a microbiome score and interpreting the same in relation to cancers. In embodiments, the present invention relates to using artificial intelligence with respect to the same.

### BACKGROUND OF THE INVENTION

Throughout this application various publications are referred to, including by number in square brackets. Full citations for these references may be found at the end of the specification. The disclosures of these publications, and all patents, patent application publications and books referred to herein, are hereby incorporated by reference in their entirety into the subject application to more fully describe the art to which the subject invention pertains.

The microbiome describes the sum of microorganisms and their genetic information that play a role in human microbial ecology [1, 2]. While microbial genetic profiles intuitively can affect the gut, associations with organs outside the gut has drawn attention and criticism [3]. The concept of a gut microbiome-based signature to predict disease outside the intestine is gaining traction, most recently in liver cirrhosis, Alzheimer's disease, lung cancer, pancreatic cancer, and others [4-7].

One of the most extreme examples of gut alteration (dysbiosis) linked to prostate cancer is inflammatory bowel diseases (IBD), which has a four-fold (HR: 4.84 (95% CI 3.34-7.02), p<0.001) increased risk of clinically significant PCa among age- and race-matched men (n=1000) [8]. Diet and obesity have associations with prostate cancer and can significantly influence the gut microbiome [9-11]. These studies provide indirect evidence that microbes may be inducing a pro-carcinogenic systemic response [12-15]. Considering the decline in prostate cancer screening with subsequent rise in metastatic disease, a PSA-independent biomarker indicating PCa risk would be advantageous.

### SUMMARY OF THE INVENTION

A gut microbiome signature has been developed herein for cancers, including prostate cancer (PCa), using a prostate specific antigen (PSA)-independent, gut microbiome-based PCa biomarker, which can be used for diagnosis, suitability for screening, treatment decisions and as part of a treatment method. Serial sampling using the methods can be employed to track efficacy of treatments or activities, and inform future screening or indicate suitability of different treatments.

A method is provided of determining whether a gut microbiome sample from a human subject is associated with a clinically significant cancer comprising:
(a) obtaining or having obtained a gut microbiome-containing sample from a human subject;
(b) performing a genetic analysis on the gut microbiome-containing sample so as to determine a (i) presence or not and (ii) abundance value of each of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g Finegoldia;
   p_Proteobacteria;
   f_Bacteroidaceae; and
   c_Deltaproteobacteria;
(c) generating a numerical microbiome score from the genetic analysis performed in (b);
(d) comparing the numerical microbiome score generated in (c) with a predetermined reference value which delineates a cutoff between (i) clinically significant cancer and (ii) low-risk cancer/negative biopsy, so as to determine if the gut microbiome sample is associated with a clinically significant cancer,
wherein a numerical microbiome score equal to or greater than the predetermined reference value indicates the sample comes from a human subject at risk of clinically significant cancer and a numerical microbiome score less than the predetermined reference value indicates the sample comes from a human subject with low-risk of cancer/negative biopsy.

A method is provided of determining whether a gut microbiome sample from a human subject is associated with a cancer risk comprising:
(a) obtaining or having obtained a gut microbiome-containing sample from a human subject;
(b) performing a genetic analysis on the gut microbiome-containing sample so as to determine a (i) presence or not and (ii) abundance value of each of the following taxa:
g_Oscillospora:
g_Lachnospira;
f_Corynebacteriaceae;
g_Corynebacterium;
o_Actinomycetales;
f_Paraprevotellaceae;
g_Eubacterium;
g_Peptoniphilus;
o_Enterobacteriales;
c_Erysipelotrichi;
c_Gammaproteobacteria;
g_Finegoldia;
p_Proteobacteria;
f_Bacteroidaceae; and
c_Deltaproteobacteria;
(c) generating a numerical microbiome score from the genetic analysis performed in (b);
(d) comparing the numerical microbiome score generated in (c) with a predetermined reference value which delineates a cutoff between (i) a cancer risk and (ii) no current risk of cancer, so as to determine if the gut microbiome sample is associated with a cancer risk,
   wherein a numerical microbiome score equal to or greater than the predetermined reference value indicates the sample comes from a human subject at risk of a cancer and a numerical microbiome score less than the predetermined reference value indicates the sample comes from a human subject not currently at risk of cancer.

A method is provided of identifying a subject as in need of a cancer screening test, comprising:
(a) obtaining or having obtained a gut microbiome-containing sample from a human subject;
(b) performing a genetic analysis on the gut microbiome-containing sample so as to determine a (i) presence or not and (ii) abundance value of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) comparing the numerical microbiome score generated in (c) with a predetermined reference value which delineates a cutoff between (i) clinically significant cancer and (ii) low-risk cancer/negative biopsy, so as to determine if the gut microbiome sample is associated with a clinically significant cancer,
   wherein a numerical microbiome score equal to or greater than the cutoff indicates the sample comes from a human subject in need of a cancer screening test and a numerical microbiome score less than the cutoff indicates the sample comes from a human subject not in need of a cancer screening test.

A method is provided of treating a prostate cancer in a subject comprising:
(A) obtaining, or having obtained, a diagnosis of the subject as having a clinically significant prostate cancer by a method comprising, on a gut microbiome-containing sample from a human subject,
   a) performing a genetic analysis on the sample so as to determine a (i) presence or not and (ii) abundance value of each of the following taxa:
      g_Oscillospora;
      g_Lachnospira;
      f_Corynebacteriaceae;
      g Corynebacterium;
      o_Actinomycetales;
      f_Paraprevotellaceae;
      g_Eubacterium;
      g_Peptoniphilus;
      o_Enterobacteriales;
      c_Erysipelotrichi;
      c_Gammaproteobacteria;
      g_Finegoldia;
      p_Proteobacteria;
      f Bacteroidaceae; and
      c_Deltaproteobacteria;
   b) generating a numerical microbiome score from the genetic analysis performed in a),
   c) comparing the numerical microbiome score generated in b) with a predetermined reference value which delineates a cutoff between (i) clinically significant cancer and (ii) low-risk cancer/negative biopsy, so as to determine if the gut microbiome sample is associated with a clinically significant cancer,
   wherein a numerical microbiome score equal to or greater than the predetermined reference value indicates the sample comes from a human subject at risk of a clinically significant cancer and a numerical microbiome score less than the predetermined reference value indicates the sample comes from a human subject with low-risk of cancer/negative biopsy,
(B) treating a subject, having a diagnosis in (A) as having a clinically significant prostate cancer, comprising administering an androgen suppression therapy, abiraterone, external beam radiation (with or without brachytherapy), and/or radical prostatectomy (with or without pelvic lymph node dissection) to the subject.

A kit is provided comprising (a) primer pairs and (b) one or more reagents, wherein in (a) the primer pairs consists essentially of primer pairs for all of:
g_Oscillospora;
g_Lachnospira;
f_Corynebacteriaceae;
g_Corynebacterium;
o_Actinomycetales;
f_Paraprevotellaceae;
g_Eubacterium;
g_Peptoniphilus;
o_Enterobacteriales;
c_Erysipelotrichi;
c_Gammaproteobacteria;
g_Finegoldia;
p_Proteobacteria;
f_Bacteroidaceae; and
c_Deltaproteobacteria.

A kit comprising primers or probes specific for the 16s V1V2 variable regions of each of the following, and a human fecal sample:
k_Bacteria.p_Bacteroidetes;
k_Bacteria.p_Proteobacteria;
k_Bacteria.p_Actinobacteria.c_Actinobacteria;
k_Bacteria.p_Bacteroidetes.c_Bacteroidia;
k_Bacteria.p_Firmicutes.c_Bacilli;
k_Bacteria.p_Firmicutes.c_Erysipelotrichi;
k_Bacteria.p_Proteobacteria.c_Deltaproteobacteria:
k_Bacteria.p_Proteobacteria.c_Epsilonproteobacteria;
k_Bacteria.p_Proteobacteria.c_Gammaproteobacteria;
k_Bacteria.p_Actinobacteria.c_Actinobacteria.o_Actinomycetales;
k_Bacteria.p_Bacteroidetes.c_Bacteroidia.o_Bacteroidales;
k_Bacteria.p_Firmicutes.c_Bacilli.o_Lactobacillus;
k_Bacteria.p_Firmicutes.c_Erysipelotrichi.o_Erysipelotrichales;
k_Bacteria.p_Proteobacteria.c_Deltaproteobacteria.o_Desulfovibrionales;
k_Bacteria.p_Proteobacteria.c_Epsilonproteobacteria.o_Campylobacterales;
k_Bacteria.p_Proteobacteria.c_Gammaproteobacteria.o_Enterobacteriales;
k_Bacteria.p_Actinobacteria.c_Actinobacteria.o_Actinomycetales.f_Corynebacteriaceae;
k_Bacteria.p_Bacteroidetes.c_Bacteroidia.o_Bacteroidales.f_Bacteroidaceae;
k_Bacteria.p_Bacteroidetes.c_Bacteroidia.o_Bacteroidales.f_Odoribacteraceae;
k_Bacteria.p_Bacteroidetes.c_Bacteroidia.o_Bacteroidales.f_Paraprevotellaceae;
k_Bacteria.p_Bacteroidetes.c_Bacteroidia.o_Bacteroidales.f_Porphyromonadaceae;
k_Bacteria.p_Bacteroidetes.c_Bacteroidia.o_Bacteroidales.f_Rikenellaceae;
k_Bacteria.p_Firmicutes.c_Clostridia.o_Clostridiales.f_Ruminococcaceae;
k_Bacteria.p_Firmicutes.c_Clostridia.o_Clostridiales.f_Tissierellaceae;
k_Bacteria.p_Firmicutes.c_Erysipelotrichi.o_Erysipelotrichales.f_Erysipelotrichaceae;
k_Bacteria.p_Proteobacteria.c_Deltaproteobacteria.o_Desulfovibrionales.f_Desulfovibrionaceae;
k_Bacteria.p_Proteobacteria.c_Epsilonproteobacteria.o_Campylobacterales.f_Campylobacteraceae;
k_Bacteria.p_Proteobacteria.c_Gammaproteobacteria.o_Enterobacteriales.f_Enterobacteriaceae;
k_Bacteria.p_Actinobacteria.c_Actinobacteria.o_Actinomycetales.f_Corynebacteriaceae.g_Corynebacterium;
k_Bacteria.p_Bacteroidetes.c_Bacteroidia.o_Bacteroidales.f_Bacteroidaceae.g_Bacteroides;
k_Bacteria.p_Firmicutes.c_Clostridia.o_Clostridiales.f_Lachnospiraceae.g_Coprococcus;
k_Bacteria.p_Firmicutes.c_Clostridia.o_Clostridiales.f_Lachnospiraceae.g_Lachnospira;
k_Bacteria.p_Firmicutes.c_Clostridia.o_Clostridiales.f_Ruminococcaceae.g_Oscillospira;
k_Bacteria.p_Firmicutes.c_Clostridia.o_Clostridiales.f_Tissierellaceae.g_Finegoldi;
k_Bacteria.p_Firmicutes.c_Clostridia.o_Clostridiales.f_Tissierellaceae.g_Peptoniphilus;
k_Bacteria.p_Firmicutes.c_Clostridia.o_Clostridiales.f_Veillonellaceae.g_Dialister; k_Bacteria.p_Firmicutes.c_Erysipelotrichi.o_Erysipelotrichales.f_Erysipelotrichaceae.g_Eubacterium;
k_Bacteria.p_Proteobacteria.c_Epsilonproteobacteria.o_Campylobacterales.f_Campylobacteraceae.g_Campylobacter; and
k_Bacteria.p_Proteobacteria.c_Gammaproteobacteria.o_Pasteurellales.f_Pasteurellaceae.g_Haemophilus.

In embodiments, the primers or probes comprise pairs for each taxa.

A method is provided for determining cancer risk using a computer system comprising one or more processors operably connected to one or more memories, the method comprising:
(a) accessing, by the one or more processors, from the one or more memories, digitized sequence data associated with a gut microbiome-containing sample;
(b) generating, by the one or more processors, 16S rRNA sequence data based on the digitized sequence data;
(c) generating, by the one or more processors, a normalized matrix representing (i) presence or not and (ii) abundance of taxa associated with the 16S rRNA sequence data;
(d) selecting, by the one or more processors, prevalence data in the normalized matrix which is associated with specific taxonomic features;
(e) obtaining, by an artificial intelligence module of the computer system comprising at least one artificial intelligence model, the prevalence data;
(f) generating an output, by the artificial intelligence module comprising the at least one artificial intelligence model trained using a training set, based on a query wherein:
   i) the query comprises the prevalence data;
   ii) the output comprises a numerical microbiome score;
   iii) the training set comprises data representing the prevalence of the specific taxonomic features and associated cancer development in patients.

A method for determining whether a gut microbiome sample from a human subject is associated with a risk of a future prostate cancer or risk of a future clinically significant prostate cancer comprising:
(a) obtaining, or having obtained, a gut microbiome-containing sample from a human subject;
(b) performing a genetic analysis on the gut microbiome-containing sample so as to determine a (i) presence or not and (ii) abundance value of each of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) comparing the numerical microbiome score generated in (c) with a predetermined reference value which delineates a cutoff between (i) risk of future prostate cancer or at risk of a future clinically significant prostate cancer and (ii) low-risk of future prostate cancer/negative biopsy, so as to determine if the gut microbiome sample is associated with a risk of future prostate cancer or risk of a future clinically significant prostate cancer,
wherein a numerical microbiome score equal to or greater than the predetermined reference value indicates the sample comes from a human subject at risk of future prostate cancer or risk of a future clinically significant prostate cancer, and a numerical microbiome score less than the predetermined reference value indicates the sample comes from a human subject with low-risk of future prostate cancer/negative biopsy.

A method for reducing a risk of a prostate cancer or risk of a future prostate cancer or clinically significant prostate cancer comprising determining, or receiving a determination that, a human subject is at risk of a future prostate cancer by a method described herein, and then administering to the subject one or more agents effective to modify the numerical microbiome score of the gut microbiome of the subject so as to thereby reduce a risk of a future prostate cancer or risk of a future clinically significant prostate cancer in the subject.

### BRIEF DESCRIPTION OF THE DRAWINGS

Exemplary embodiments of the present invention will be described with references to the below referenced accompanying figures.

FIG. 1: CONSORT diagram. On the left we display the biopsy cohort who largely was recruited from the San Antonio Audie Murphy VA as part of a genitourinary tissue bank collection before prostate biopsy. The second group on the right is the men presenting for their follow up within the San Antonio Biomarkers of Risk cohort that undergoes standardized prostate cancer screening. SABOR provides yearly screening and communicates these results to their primary care physician and urologist as part of a community care collaboration. PCa Dx=prostate cancer diagnosis, GG1=grade group 1, GG2+=greater than grade group 2 prostate cancer.

FIG. 2: Line graph showing biologic gradient of PRIMUS (also referred to herein as the cancer model). We divided the PRIMUS score (also referred to as the microbiome score herein) into deciles with color coded bar graph to demonstrate the representative score. We provide the proportion of any prostate cancer (% cancer) and proportion of GG2+ (% GG2) prostate cancer in the chart. The proportion are also represented in the line graph showing increasing risk of either cancer with rising PRIMUS Score. The final two rows of the table show the raw number of patients the make up the proportions.

FIGS. 3A-3I: Association with prostate cancer detection and performance. Operational performance of the microbiome score. (3A) Training and validation. The ROC-AUC curve depicts training (2/3) and validation (1/3) model development with associated histograms for cancer and no cancer, we show that this validation techniques of the score yield significant results. (3B) 10-fold cross-validation. An alternative strategy for model testing involved 10-fold cross-validation during microbiome score development compared to the hold-out validation due to overfitting of 10-fold cross-validations. (3C) Decision analysis curve compared to that of PSA. The decision analysis curve showed that the use of the microbiome score for predicting prostate cancer had the greatest benefit relative to PSA level, biopsy all or biopsy none at thresholds ranging from 10% to 35%. Higher PSA concentrations had a greater net benefit above 35%. (3D) ROC curve analysis of the ability of the microbiome score to predict GG2+ prostate cancer. A microbiome score of 0.25 was used as the threshold to call a positive test and was associated with a sensitivity, specificity, PPV and NPV of 84.8%, 46.9%, 22.2% and 94.5%, respectively. We then used the AUC curve to test the scores performance for the association with clinically significant cancer (GG2+) and noted a similar and significant curve compare the detection of any prostate cancer. (3E) 10-fold cross-validation for GG2+. We again secondarily tested an ROC curve using 10-fold cross validation noting an AUC of 0.712 (95% CI 0.708-0.712) to compliment the hold-out validation. (3F) Decision analysis curve for GG2+ compared to PSA. The decision analysis curve showed that the ability of the microbiome score to predict GG2+ prostate cancer was similar to that of PSA in the lower PSA categories, biopsy all or biopsy none at thresholds with a limit around the 25% threshold. (3G) Correlations between the cross-validation and training/validation models. There was a strong correlation (r=0.79, p<0.001) between the scores. (3H) Association between PPV or NPV and PCa detection or GG2+PCa detection, by decile. The PRIMUS score was divided equally into 10 groups (deciles). The increasing PPV and decreasing PPV per decile show the biological gradient of the score. (3I) Microbiome score alluvial plot. Alluvial plot showing microbiome score scores in relation to a range of important serum PSA level cutoffs and outcomes used to develop and test the microbial score. (3B). The PRIMUS score cut off of 0.25 will divide the patients into a positive or negative test. These then flow into the “case” (i.e., cancer) or “control” (i.e., no cancer).

FIGS. 4A-4B: (4A) Time to any PCa diagnosis. A Kaplan-Meier plot showed decreased prostate cancer-free survival in patients with a baseline PRIMUS score of 0.25 or greater (hazard ratio (HR) 3.97, 95% CI 2.14-7.36, p<0.001). (4B) Time to GG2+PCa diagnosis. A Kaplan-Meier plot showed decreased GG2+ prostate cancer-free survival in patients with a baseline microbiome score of 0.25 or greater (HR 11.12, 95% CI 1.42-87.17; p<0.001).

FIGS. 5A-5C: External validation with calculation of the PRIMUS score from patients with inflammatory bowel disease noting modifiability in a high-risk cohort. (5A) We obtained 16S rRNA gene DNA sequences from external studies the human microbiome project 2 (HMP2) (top two box and whisker plots) and Crother's et al. study of FMT in IBD (bottom 3 figures). We calculated the PRIMUS score for PCa and compared the scores of patients with IBD (red) and without IBD (blue). The box and whisker plots in the left subpanel show microbiome score scores of women with and without IBD, which were not significantly different (Student's t test, p=0.98). On the right, scores were significantly higher among men with IBD (p=0.0024). (5B) Next, we explored the microbiome score in IBD patients who did or did not undergo fecal microbiota transplantation (FMT). We found lower microbiome scores in donor stool samples than in either the international or control stool samples from patients with IBD (both p<0.001). A comparison of the microbiome score scores 25-35 days post FMT revealed that the intervention group had similar low microbiome scores (p=0.12), while the score of the placebo group was significantly greater than that of the intervention group (p=0.002). (5C) Microbiome score plotted continuously with a smooth line showing separation 20 days post FMT with elevation starting after the end of transplantation treatment on day 35.

FIGS. 6A-6C: Microbiome diversity profiles. (6A) Alpha diversity according to cancer status (top). Stacked bar plots showing the phylum level of microbes within each sample grouped based on cancer status. GG2+ represents men with grade group 2 or higher (intermediate to high grade) prostate cancer. GG1 represents low-risk prostate cancer. The negative biopsy group included men with a risk high enough to warrant a prostate biopsy; however, the biopsy returned negative. This group may have false negatives. The low-risk group included men who underwent prostate cancer screening at usual risk low enough to not warrant a prostate biopsy. (6B) Alpha diversity: Box and whisker plots comparing alpha diversity by cancer status. Each dot represents an individual microbiome according to the Shannon diversity index (SDI). The box represents the 50% interquartile range (IQR) of the SDI distribution, and the line represents the median SDI. There was no difference between groups (P=0.49). (6C) Beta diversity: A scatter plot based on principal coordinates analysis (PCoA) to represent microbial variation is shown. Each color dot represents a different cancer status. We noted a statistically significant difference among groups (PERMANOVA result for cancer grade status: P<0.01).

FIGS. 7A-7B: (7A) Box plots of alpha diversity by race. Box plots for White (Hispanic and non-Hispanic), Black, and Other individuals showed no significant difference in alpha diversity. (7B) Principal component analysis plot for the gut microbiome and race. PCoA plot of microbial variation by race (White, Black, Other) showed significant differences in beta diversity among groups (PERMANOVA: P<0.01).

FIGS. 8A-8B: Pipeline showing the development and analysis of the PRIMUS Microbiome Score of Cancer of the Prostate (MiSCaP). 8A The gut microbiome score development schema. The process of analysis included 16S rRNA gene V1-V2 sequencing, cleaning, OTU assignment, and normalization. After, we selected taxonomic features for the machine learning algorithm by forest plot analysis, resulting in an output score ranging from 0 to 1, with higher scores indicating a greater risk of clinically significant prostate cancer. Microbial analysis to select taxonomic features for analysis. We used LEfSe analysis to identify the most differentially abundant taxa. We used LDA score>2 (x-axis) (left). On the right is the representative cladogram. Machine Learning Models. Using the same 39 bacteria was used machine learning for a 10-fold cross validation and compared this to the scores of a training-validation set. 8B Features list. The features correspond to a particular microbial representative (c-class, o-order, f=family, g-genus)

FIG. 9: Microbiome scores generated from different bacterial variable regions are significantly correlated. The correlation scatter plot shows 16S rRNA gene V1-V2 sequence scores on the x-axis and the microbiome score generated via the 16S rRNA gene V3-V4 sequence from the same samples on the y-axis (Pearson correlation coefficient=0.77, p<0.001).

FIG. 10: Microbiome score scores correlate with PSA levels. Scatter plot with log (PSA level) on the y-axis and the microbiome score on the x-axis showing good correlation between values (r=0.12, p<0.001).

FIGS. 11A-11B: Microbiome model using GG2+ as the endpoint. A. Creation and validation of the GG2+ microbiome score. The same methods used to create our microbiome score were implemented for all prostate cancer grades compared to no prostate cancer in order to create a new microbiome score with the endpoint comprising only clinically significant prostate cancer (i.e., GG2+). We again used a trained and validated model and compared it to a 10-fold cross-validation technique. These two scores showed excellent correlation (r=0.8, P<0.001). The operational characteristics at a threshold of 0.158 included a sensitivity, specificity, negative predictive value, and positive predictive value of 72.2%, 58.9%, 24.1%, and 92.2%, respectively. However, the score was not predictive of future risk of GG2+ prostate cancer (HR 1.39, p=0.45; data not shown). The new models focused only on the GG2+ correlation with the original score. Three scatter plots representing a second, separately developed microbiome score focused only on GG2+ detection compared to the original microbiome score. The original microbiome score for detecting all prostate cancers had better performance for predicting GG2+ cancer even though the newer model was built with the endpoint of GG2+ cancer. B. We constructed correlation plots for no cancer, GG1 cancer and GG2+ cancer and noted a significant correlation between scores (Spearman rho, r=0.82, p<0.001). Decision analysis curve: The decision analysis curve showed that the ability of the microbiome score to predict GG2+ prostate cancer was similar to that of PSA in the lower PSA categories, biopsy all or biopsy none at thresholds with a limit around the 25% threshold.

FIG. 12: Alterations in the microbiome score with previous antibiotic use. Box plots showing the median and 95% confidence intervals (95% CIs) of the microbiome score in patients with and without a previous biopsy (antibiotic exposure) and the resultant endpoint of cancer compared to patients without cancer. There was no difference between patients without cancer and those with cancer with or without previous antibiotic exposure as a result of prostate biopsy.

FIG. 13: Line chart showing the change in the microbiome over time. The x-axis shows the microbiome score, and the y-axis shows the time in days. Fifty-three patients without cancer were recruited from the SABOR cohort at the day 0 time point, and data for 1 or more downstream time points were plotted with colored dots. The line represents change over time. Data for the day 0 timepoint and one or more subsequent timepoints are plotted with colored dots, and the line represents the change over a median of 1.17 years (IQR: 0.99-1.34 years). Overall, the microbiome composition did not significantly change over the time frame measured (paired t test, p=0.833). The gray dots and lines represent scores that started below the threshold of 0.25 (n=25, 47.2%); the yellow dots/lines represent scores that started below the threshold and were identified as above the threshold at a later date without intervention (n=12, 22.6%); the purple dots/lines represent scores that started above the threshold and were identified as below the threshold without intentional intervention (n=7, 13.2%); and the red dots/lines represent scores that started above and remained above the threshold (n=9, 17.0%). None of the subjects developed prostate cancer during the follow-up period, although 6 subjects underwent prostate biopsy. The longitudinal cohort was diverse, with 30% (n=16) Black, 38% (n=20) Latino/Hispanic, and 32% (n=17) White individuals.

FIG. 14: Predicted metabolic functional analysis between high (red/bottom) and low (blue/top) PRIMUS scores. Differential abundance analysis identified the most differentially abundant pathways between high and low PRIMUS score groups. Comparison of PICRUSt profiles utilized the Mann-Whitney test with FDR P-value adjustment. The bar plot figure describes the top categories related to cancer or metabolism enriched in either high (red) or low (blue) PRIMUS score. All results shown achieved FDR adjusted P-values <1 e-3 with a relative fold change exceeding 1.2. Relative fold change values were capped at 10.

FIGS. 15A-15B: Polyketide Synthase (PKS) as an added variable to the PRIMUS score. (15A) Time to any PCa diagnosis with PKS as an added variable. A Kaplan-Meier plot showed the highest increasing incidence of prostate cancer in patients with a baseline PRIMUS score of 0.25 or greater and pks+ however did not show a statistically significant difference compared to the PRIMUS^{high} pks⁻ group (p=0.26). (15B) Time to GG2+ PCa diagnosis with PKS as an added variable. A Kaplan-Meier plot showed increased incidence of GG2+ prostate cancer in patients with a baseline microbiome score of 0.25 or greater and pks+, however was not a statistically difference when compared to PRIMUS^{high} pks⁻ (p=0.55).

FIG. 16: Consort Diagram. Sample collection of the gut microbiome was collected from 2016 to 2021 with 500 samples being collected in 2021. Several types of sequencing were used for different; however, samples underwent 16s V1-V2 sequencing and was used as the baseline sequencing method for biomarker development.

FIG. 17: Alpha diversity of cancer status (top). Standard bar plots are shown noting the phylum level of microbes within each sample grouped based on cancer status. GG2+ represents the men with grade group 2 or high (intermediate to high grade) prostate cancer. GG1 represents low risk prostate cancer. The negative biopsy group are men with a risk high enough to warrant a prostate biopsy; however, the biopsy returned negative. This group may have false negatives. The “low-risk” group are men that underwent prostate cancer screening at usual risk low enough to not warrant a prostate biopsy. Alpha diversity analysis (Bottom left): bar and whisker plots are shown that compare the cancer status groups noting. Each dot represents a person's microbiome plotted based on the Shannon Diversity Index (SDI). The box represents the 95% confidence interval, and the line represents the median SDI; however there was no difference between groups (P>0.05). Beta Diversity (Bottom Right): a scatter plot is shown based on principle coordinates analysis (PCoA) as the axis to represent microbial variation. Each color dot represents a different cancer status. A statistically significant difference between groups is noted (PERMANOVA result for cancer grade status: P<0.01).

FIGS. 18A-18C: Microbiome Score of Cancer the Prostate (MiSCaP) development and analysis. 18A. Gut microbiome score development schema. The process of analysis is shown after 16s V1-2 sequencing, cleaning, OTU assignment, and normalization. After this process taxonomic features were selected for the machine learning algorithm by forest plot analysis. At the end of the analysis the output is a score valued 0 to 1 with higher scores having more risk of clinically significant prostate cancer. 18B Microbial analysis to select taxonomic features for analysis. LEfSe analysis was selected to identify the most differentially abundant taxon. An LDA score>2 was used shown (x-axis) on the left. On the right is the representative cladogram. Cladogram of significant changes at all taxonomic levels. The root of the cladogram represents the domain bacteria. The size of node represents the abundance of taxa. 18C AUC of Microbiome Score prediction of GG2+ prostate cancer. The upper left of the figure shows the AUC of 69.7 (95% CI 69.4-69.7). The threshold used to call a positive test was a microbiome score of 0.158 that led to a sensitivity, specificity, positive predictive value (PPV) and negative predictive value (NPV) of 75.4%, 57%, 24.17% and 92.72%. The top left shows the top 15 microbes and their importance level in the model. The bottom two histograms show the microbiomes scores of those without cancer or grade group 1 prostate cancer on top (green) compared to the distribution of men with grade group 2 or higher prostate cancer on the bottom (orange).

FIG. 19: Measures of internal microbiome score robustness. Left. Correlation plot comparing the microbiome score generated from different bacterial variable regions. The correlation scatter plot shows V1V2 16s sequencing generated scores on the x axis and the microbiome score generated via V3V4 16s sequencing on the same samples. The scores are significantly correlated (Pearson correlation=0.77, p<0.001). Right. Line chart noted the change in microbiome over time. The x axis shows the microbiome score, and y axis shows time in days. N=53 subjects without cancer were recruited from the SABOR cohort with day 0 time point and 1 or more downstream time points are plotted with a colored dot and line representing the change over time. The grey dot and lines started below the GG2+ microbiome score of 0.158 (n=13, 24.5%). The yellow dot/line started below the threshold and tracked above the threshold at a later date without intervention (n=8, 15.1%). The purple dot/line started above the threshold and tracked below without intentional intervention (n=5, 9.4%). Lastly, the red dot/line started above the threshold and stayed above the threshold (n=27, 50.9%). Overall, the microbiome was not statistically different over the timeframe compared (paired T test, p=0.78). None of the subject developed prostate cancer during the follow up period although 6 subjects underwent prostate biopsy. A diverse longitudinal cohort of 30% (n=16) Black, 38% (n=20) Latino/Hispanic, and 32% (n=17) White was used.

FIG. 20: Results of the model built for any prostate cancer that includes low risk prostate cancer compared to no cancer. The model required the same base of 39 microbes as the GG2+ model; however, had a different output in machine learning random forest analysis. The features list of microbes is described in the top right of the figure. (Top Left) Using the microbiome score output threshold of 0.256 for the diagnosis of any prostate cancer. Using a training/validation set the microbiome score achieved an AUC of 0.685 (95% CI 0.674-0.686, p<0.001). In a second analysis using a 10-fold cross validation approach the microbiome score attained an AUC of 0.732 (95% CI 0.728-0.732, p<0.001). The operational characteristics of sensitivity, specificity, positive predictive value, and negative predictive value at this threshold are 84.4%, 53.9%, 41.9%, and 89.8%, respectively. A histogram of each microbiome score shown in blue for negative and red for positive for cancer. Because the two scores have different methods of generation, the outputs were compared on a scatter plot with correlation (Pearson 0.78 In univariant analysis, the microbiome score (any prostate cancer vs. no prostate cancer) was statistically significant (p<0.001) and in logistic regression had an odds ratio of 11.7 (95% CI 4.36-33.2, P<0.001).

FIGS. 21A-21B: Operational performance of the microbiome signature. (A) Microbiome Score Performance in the detection of prostate cancer. (i) Train-Validate. The ROC curve first is displayed with a training (2/3) and validation (1/3) model development with associated histograms for cancer and no cancer (AUC 0.686, 95% CI 0681-0.687, p<0.001). (ii) 10-fold cross validation. Second, the AUC is displayed for 10-fold cross validation method for microbiome signature development (AUC 0.717, 95% CI 0.713-0.717, p<0.001). The sensitivity, specificity, positive predictive value and negative predictive value are 82.7%, 52.1%, 40.2%, and 88.5%, respectively. (iii) Features of the microbiome signature. The top 10 feature characteristics of the microbiome signature with the length of each line demonstrating their relative importance. (iv) Decision analysis curve compared to PSA. The decision analysis curve shows the microbiome signature to predict any prostate cancer has the greatest benefit relative to PSA, biopsy all or biopsy none at thresholds from 10% to 35%. PSA has the greatest net benefit beyond 35%. (v) Time to any PCa. A Kaplan-Meier graph is shown noting less prostate cancer free survival in those with a baseline microbiome signature of 0.25 or greater (HR 3.97, 95% CI 2.14-7.36, p<0.001). (B). AUC of Microbiome Score prediction of GG2+ prostate cancer. (vi) ROC curve using the microbiome signature for detection of GG2+ prostate cancer. The ROC curve displays an AUC of 0.712 (95% CI 0.708-0.712). The threshold used to call a positive test was a microbiome score of 0.25 that led to a sensitivity, specificity, positive predictive value (PPV) and negative predictive value (NPV) of 84.8%, 46.9%, 22.2% and 94.5%, respectively. (vii) Time to GG2+. A Kaplan-Meier graph is shown noting less GG2+prostate cancer free survival in those with a baseline microbiome signature of 0.25 or greater (HR 11.12, 95% CI 1.42-87.17, p<0.001).

FIG. 22: Microbiome signature alluvial plot. An alluvial plot is shown for the microbiome signature in relation to a range of important PSA cut offs and outcomes used to develop and test the microbial signature.

FIGS. 23A-23C: Microbiome score is modifiable in a high-risk cohort. (A) The 16s sequences were obtained and calculated the microbiome signature for prostate cancer. The score of those with IBD (red) was compared to those without IBD (blue). On the left the microbiome signatures were compare using box and whisker plot in women and show no difference in the score (Students T, p=0.98). On the right men with and without IBD are shown noting a significantly higher signature score in men with IBD (p=0.0024). (B) The microbiome signature was then explored in IBD subjects undergoing fecal microbiota transplant or not. The donor stool had a significantly low microbiome score and compared to both groups (international and control) with IBD (both, p<0.001). Then comparing the microbiome signatures after 25-35 days of FMT the intervention group assumed a similar low microbiome score as the donors (p=0.12), while the placebo group was significantly higher than the intervention group (p=0.002). (C) When the microbiome signature is plotted continuously with a smooth line and there is a nic separation after 20 days with elevation starting after stopping the FMT at day 35.

FIG. 24: Association of genotoxin presence with cancer presence and grade. SABOR and PSA cohorts are pooled (n=388).

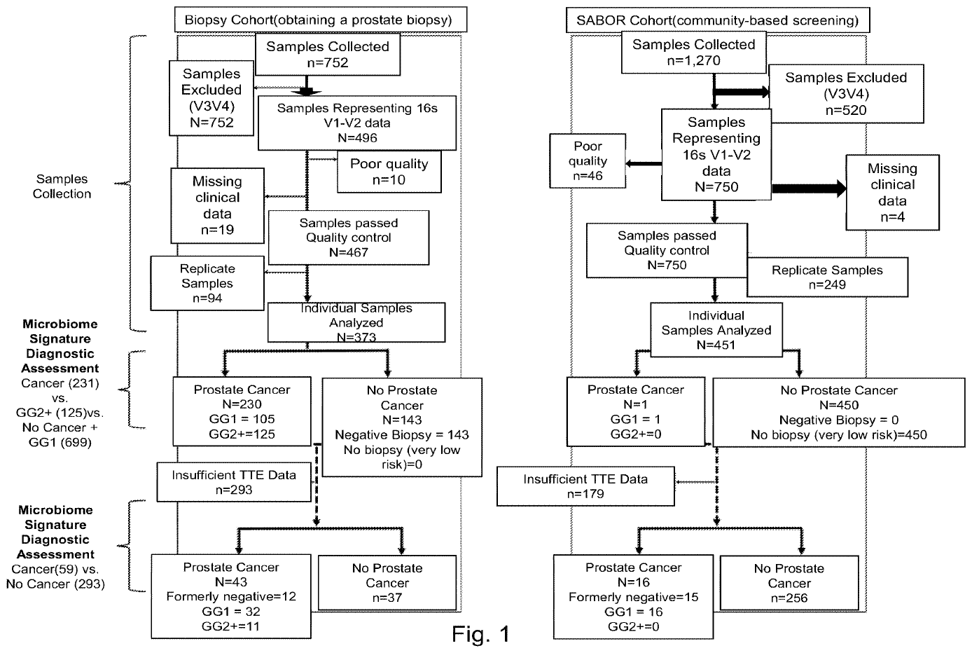

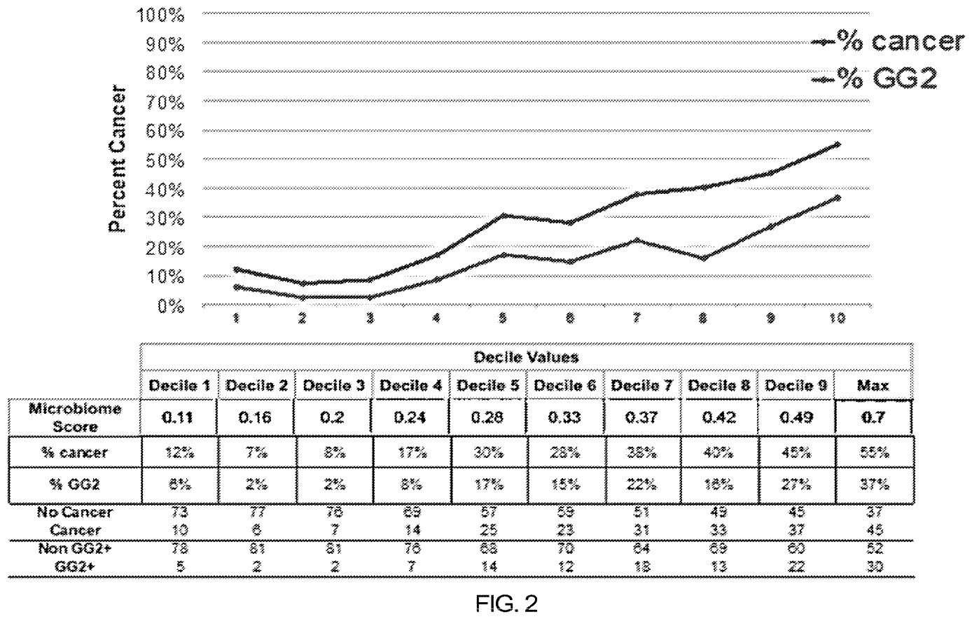

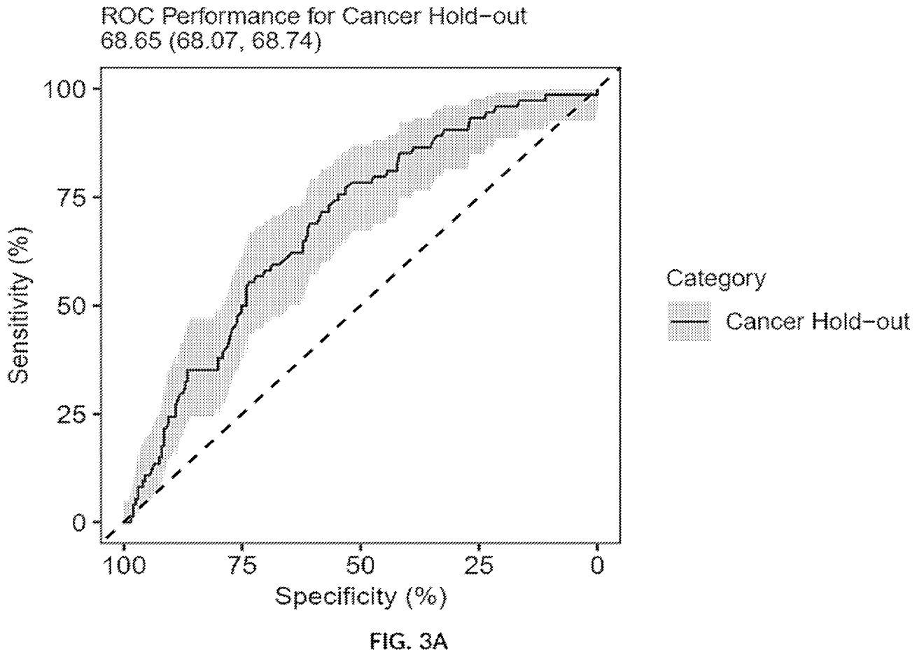

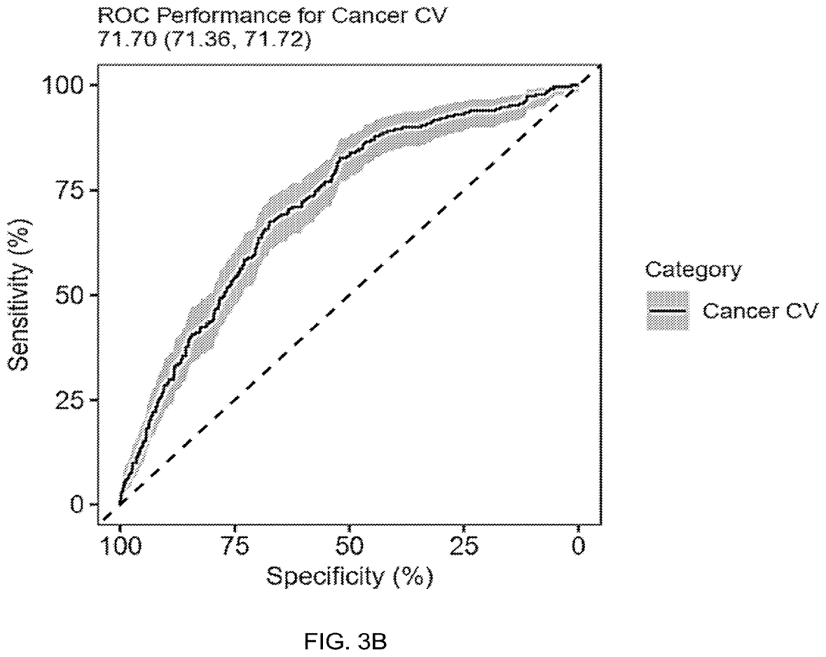

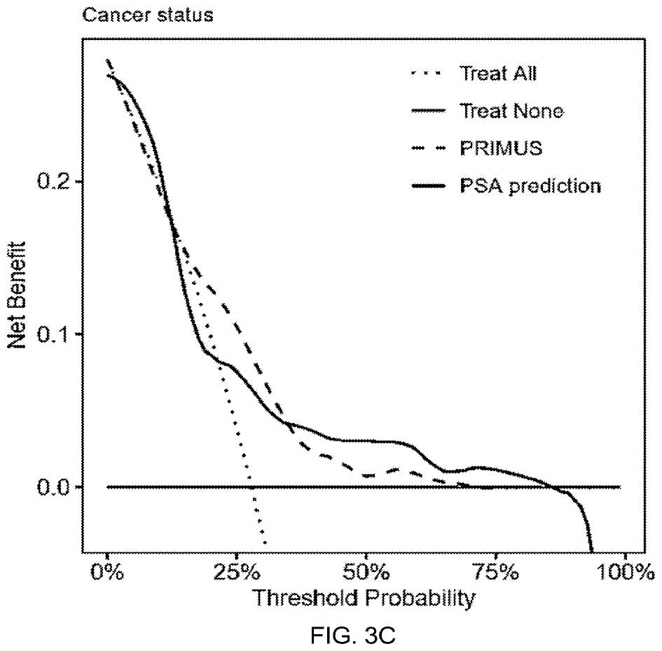

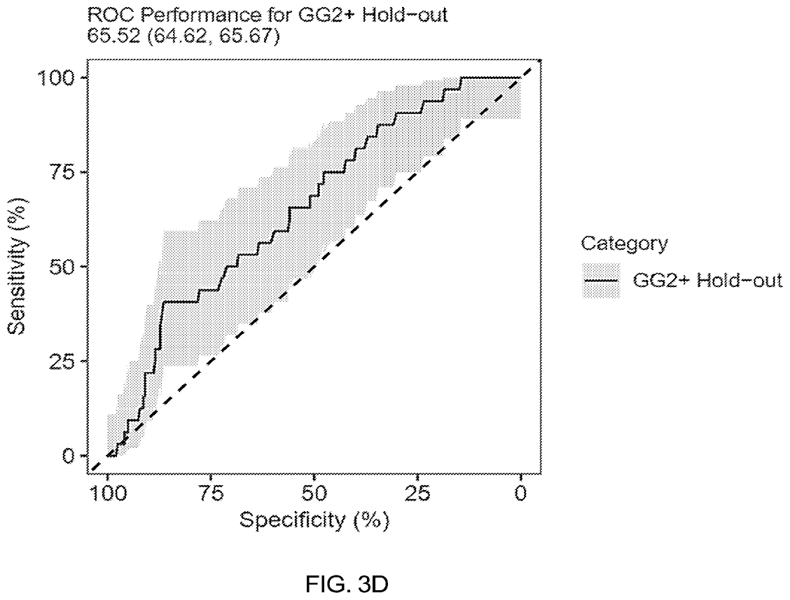

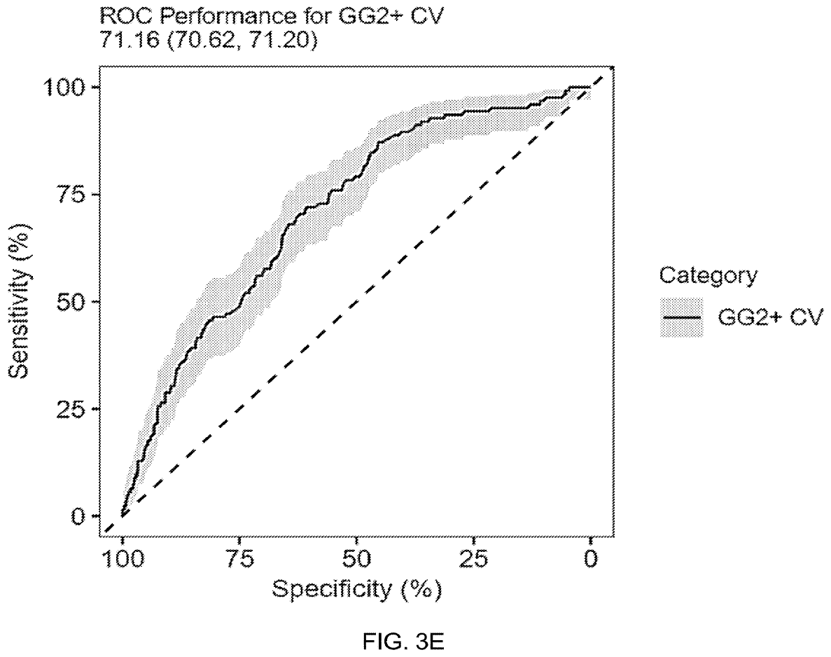

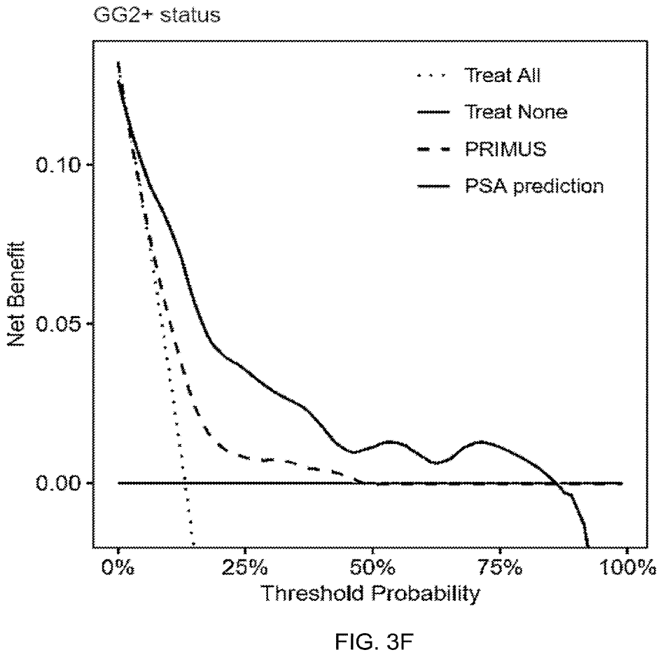

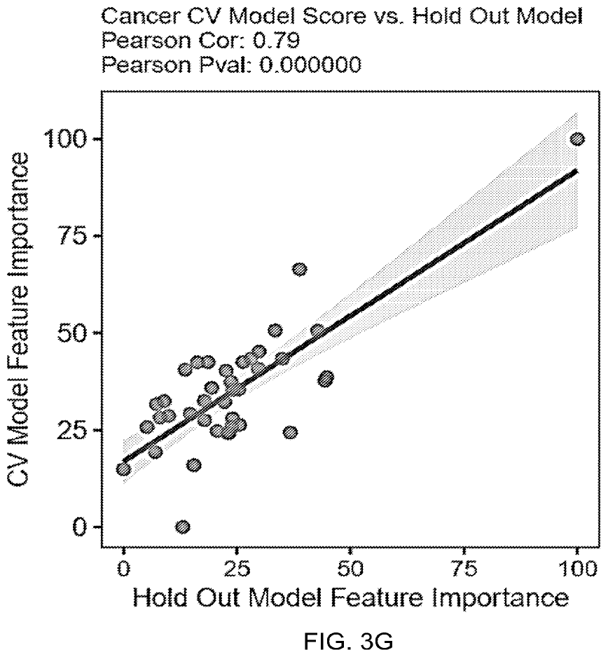

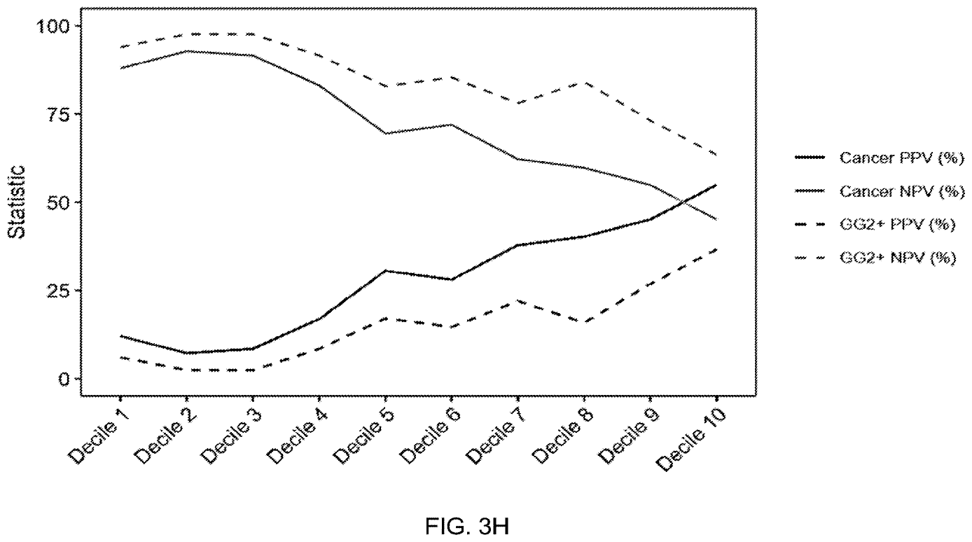

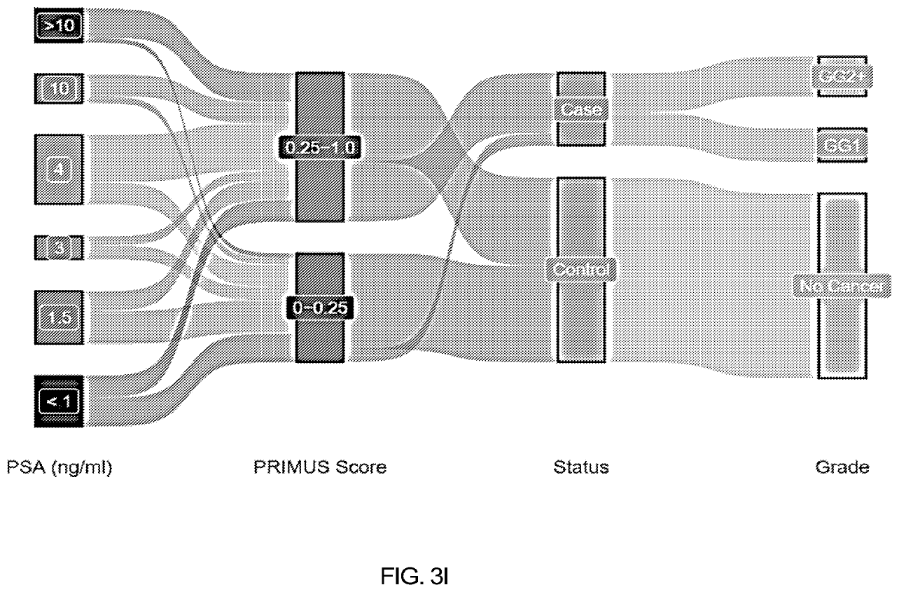

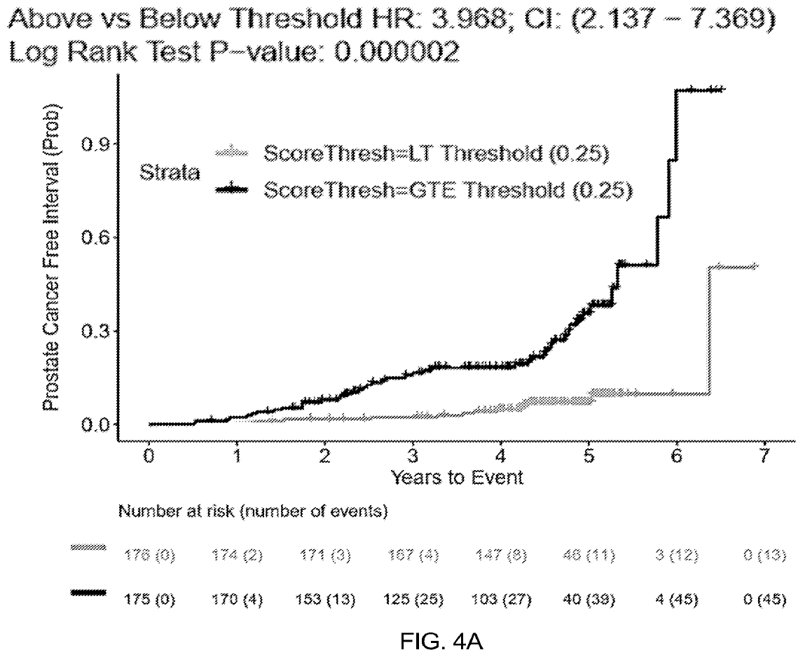

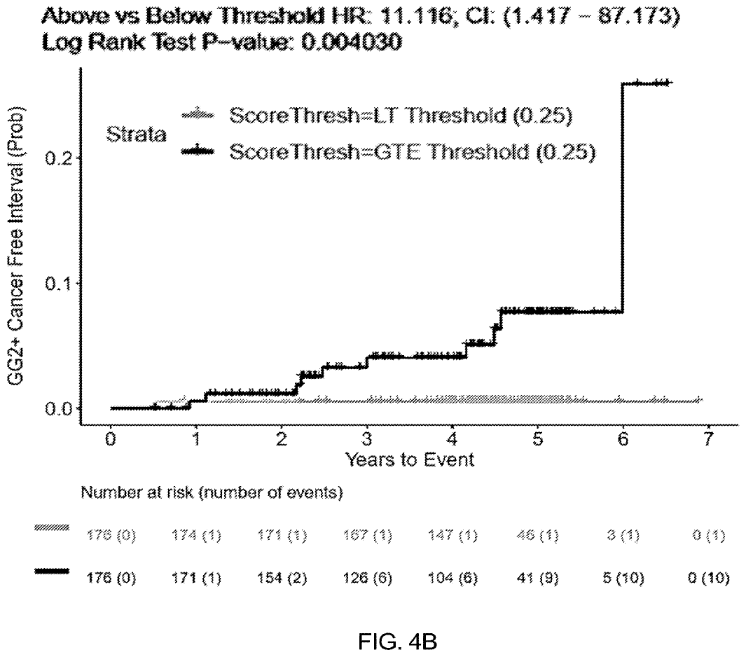

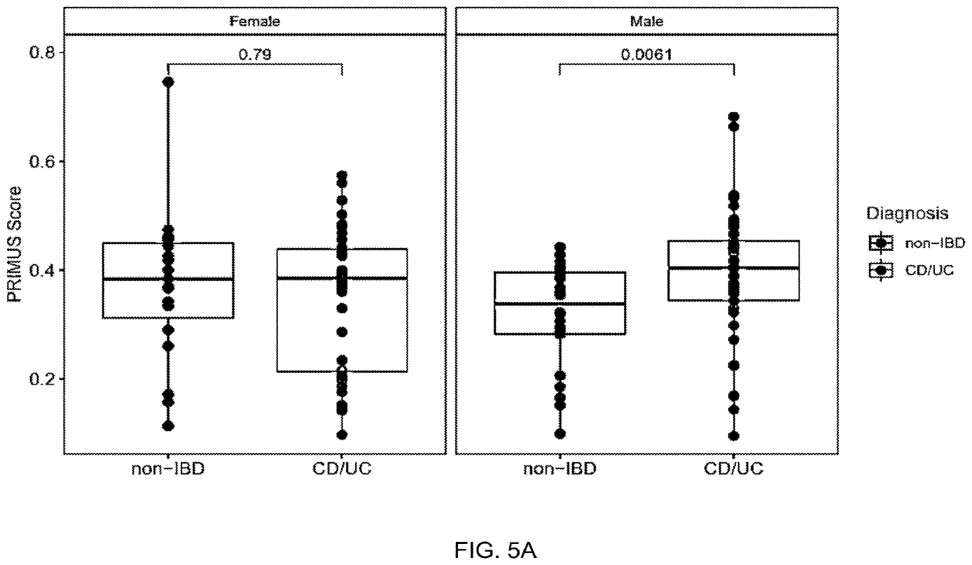

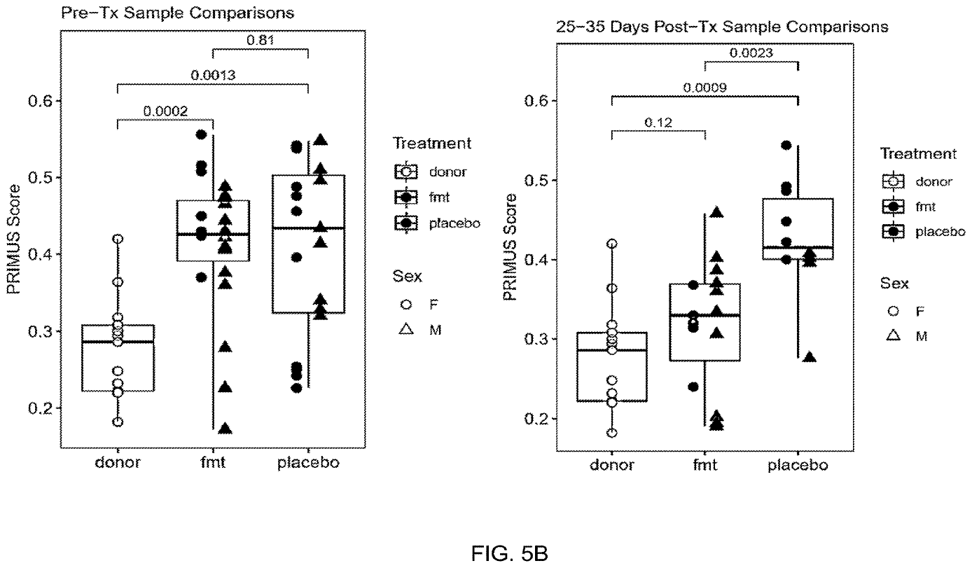

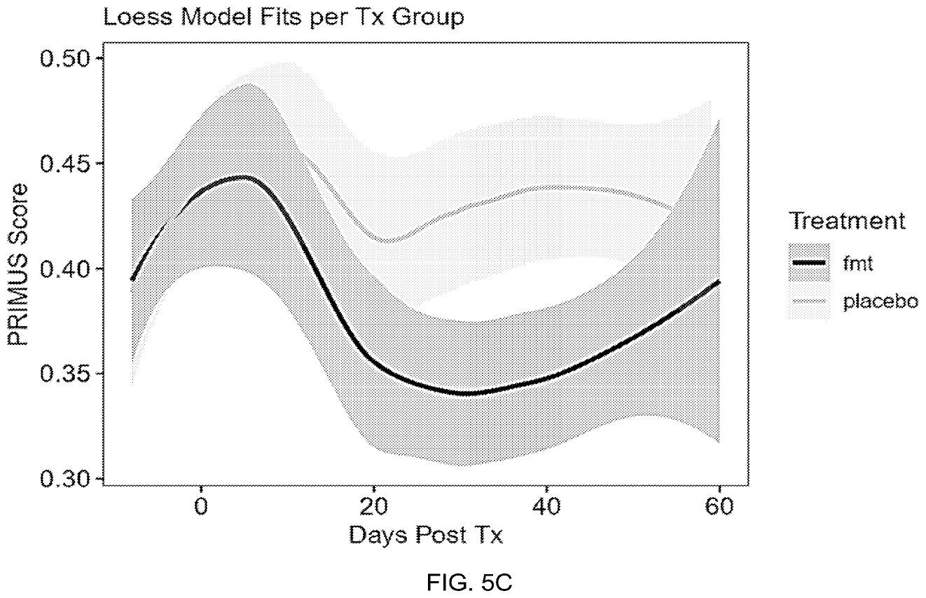

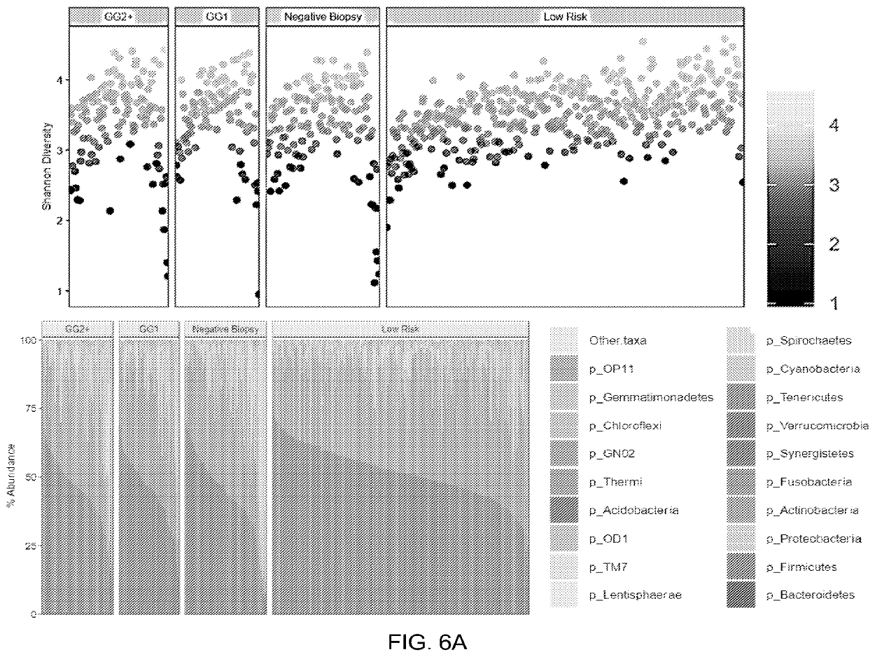

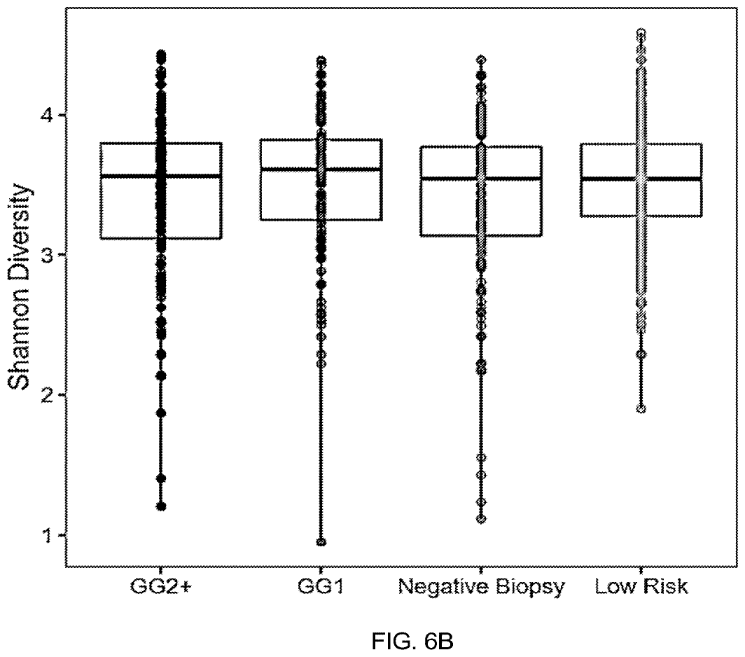

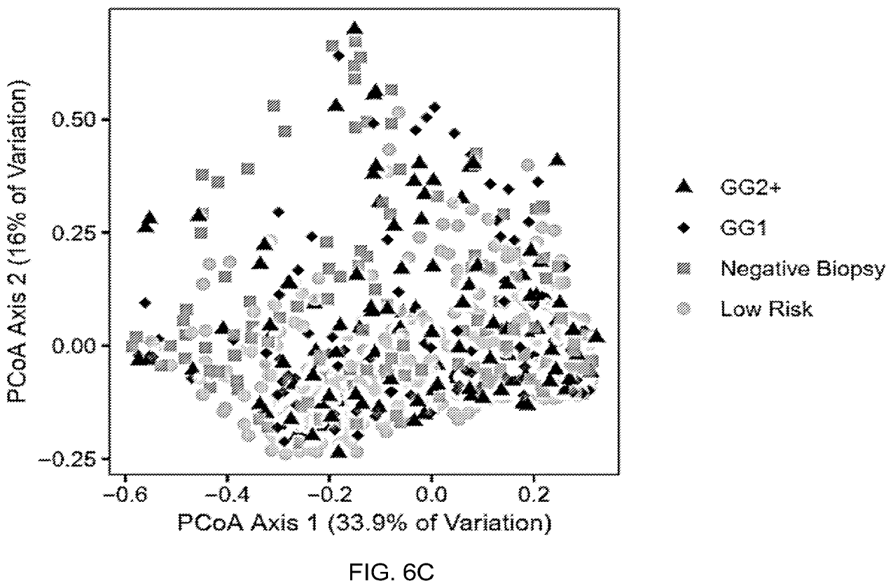

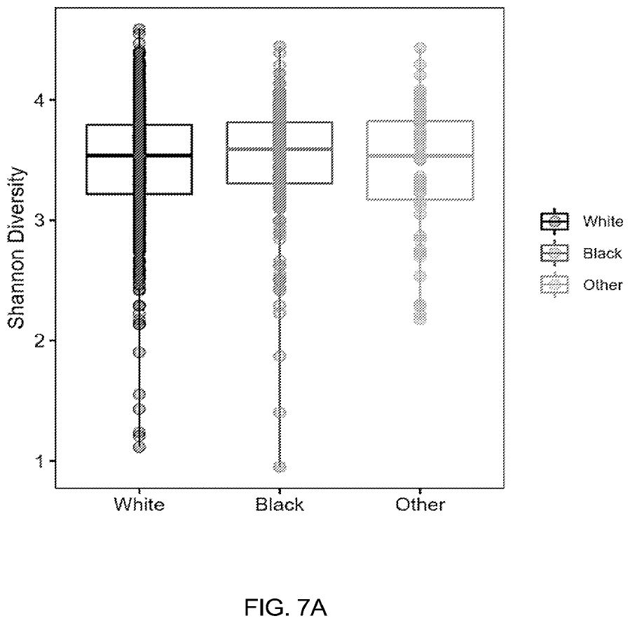

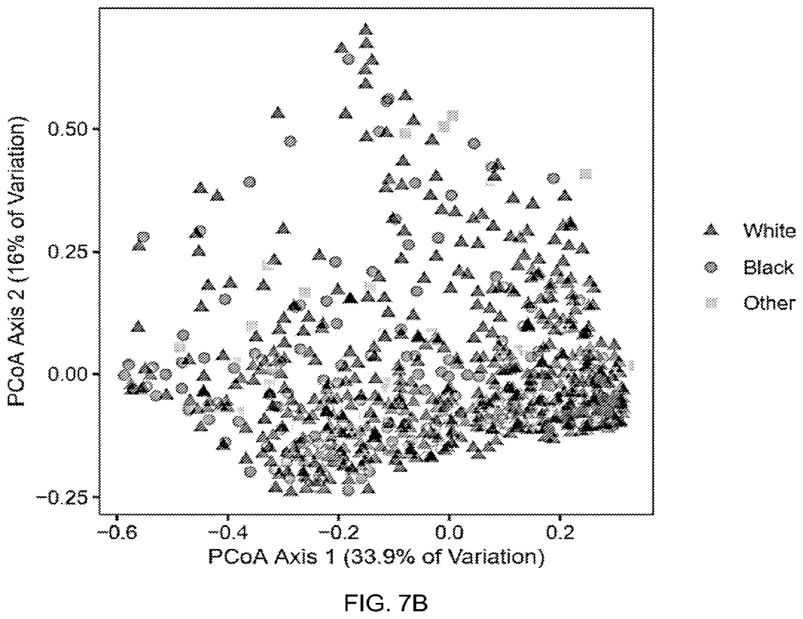

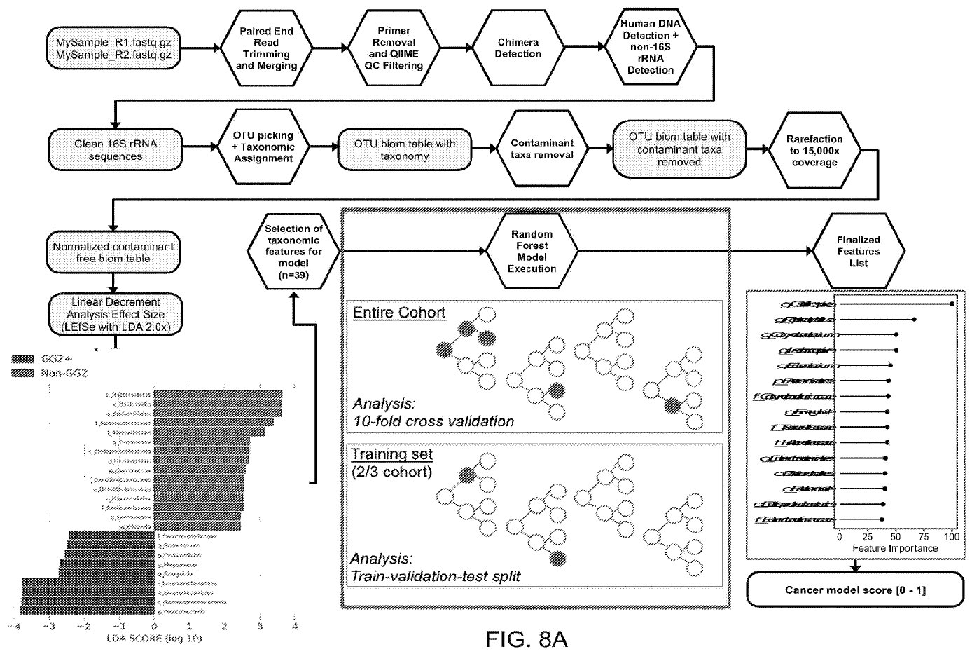

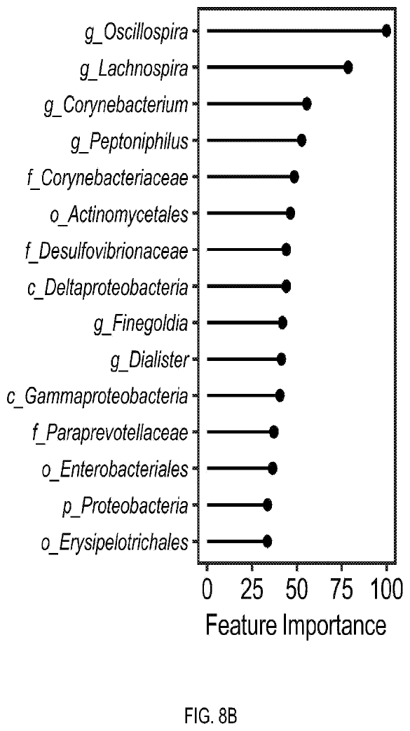

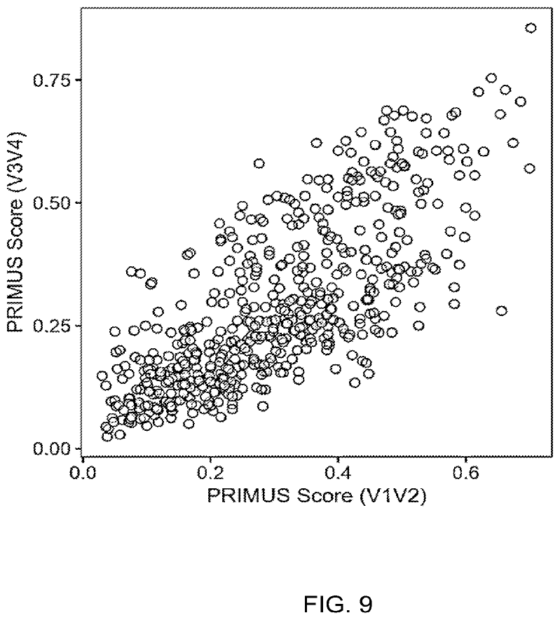

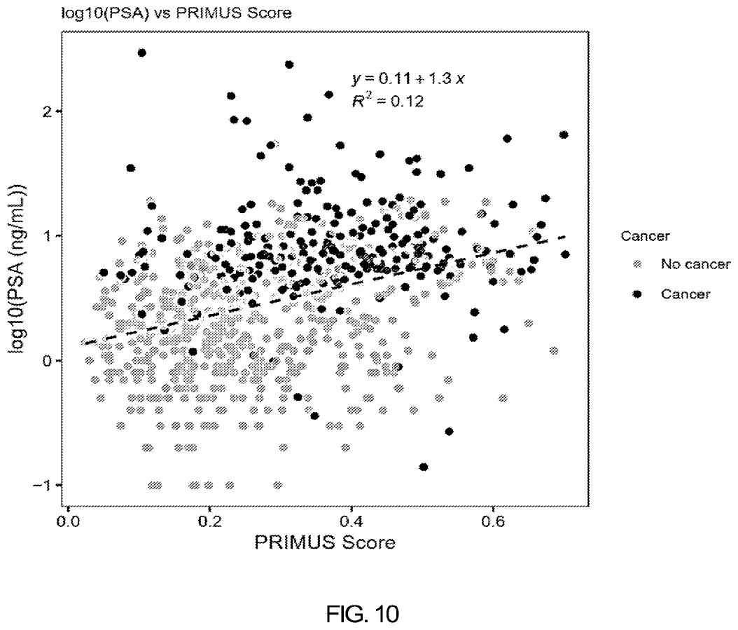

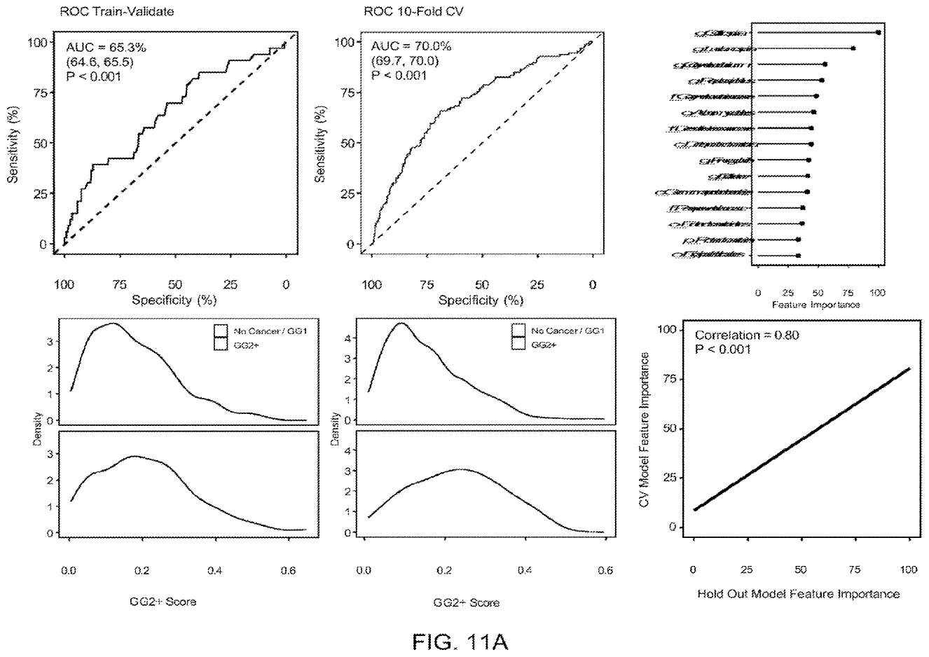

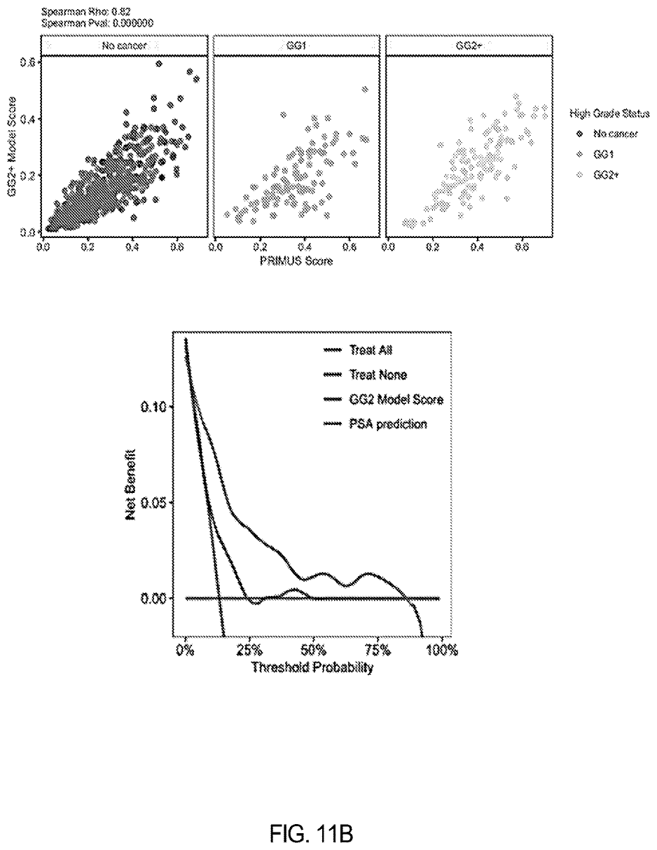

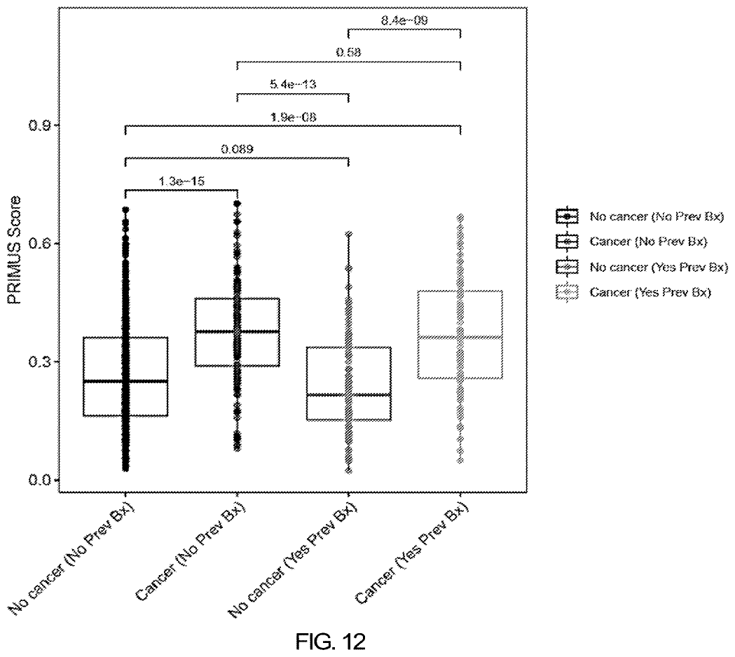

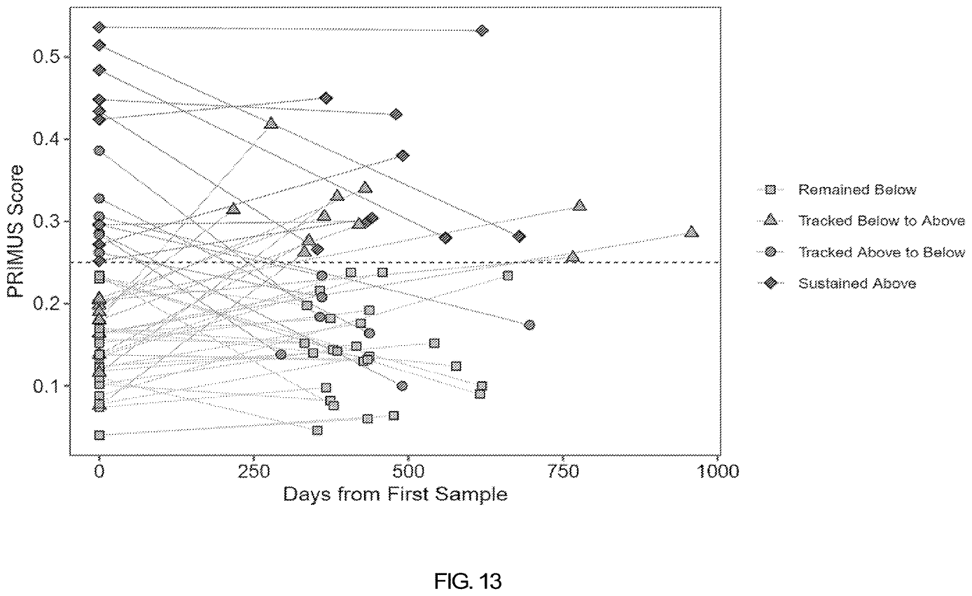

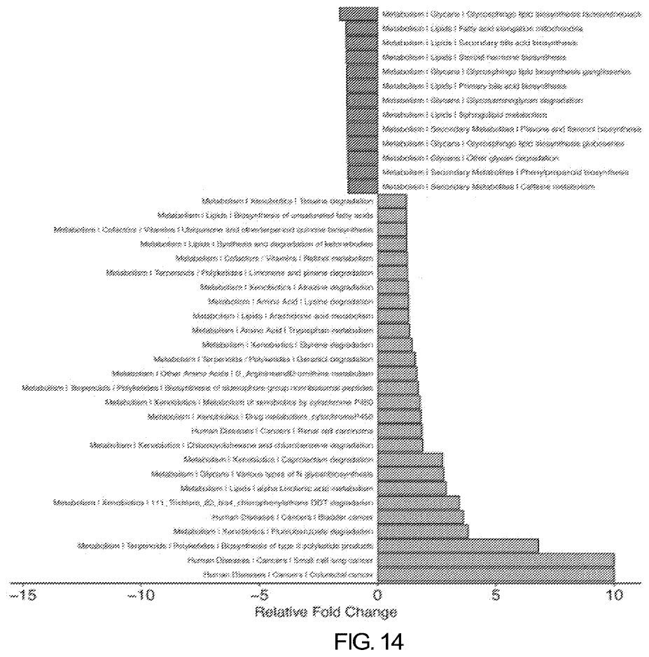

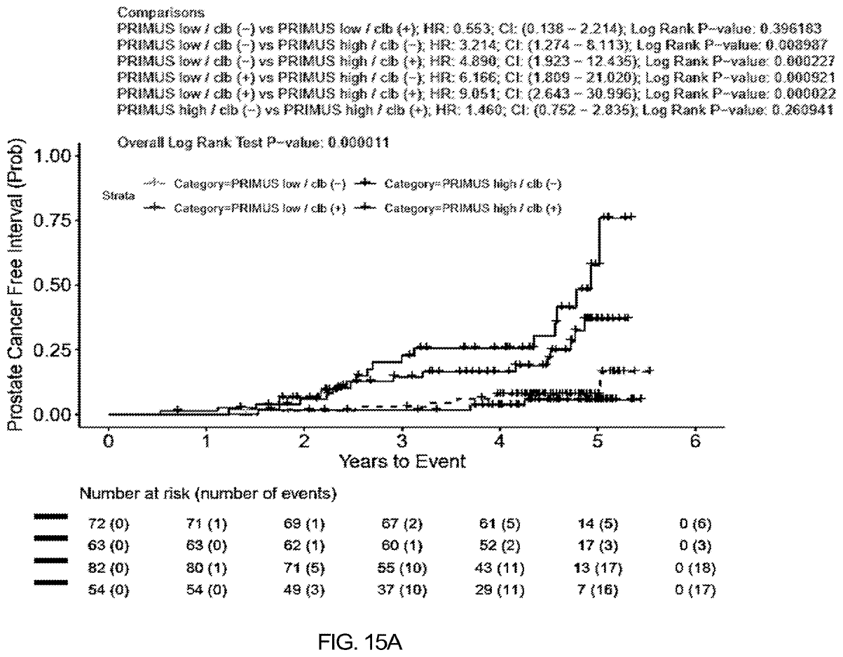

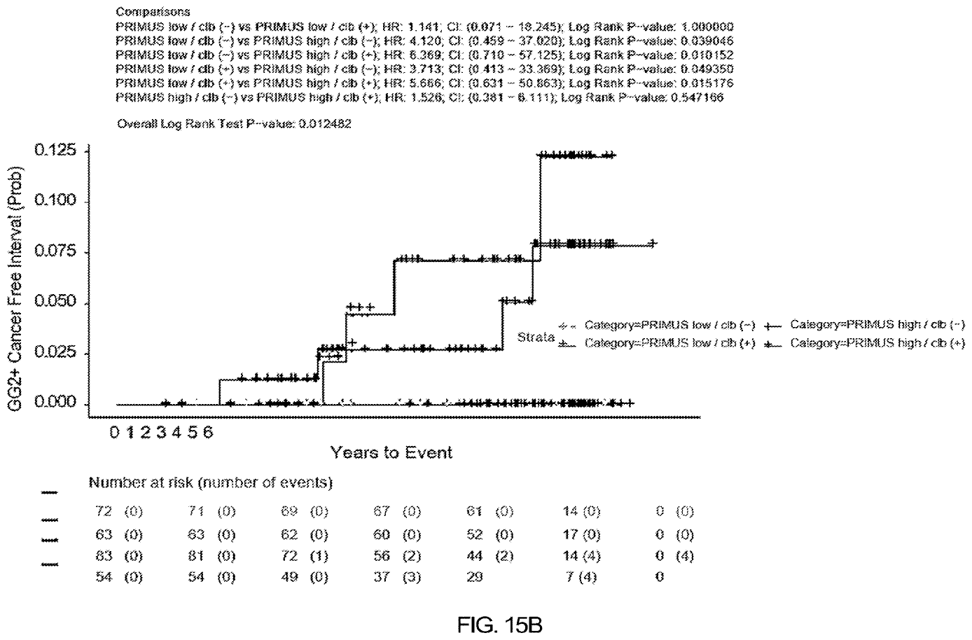

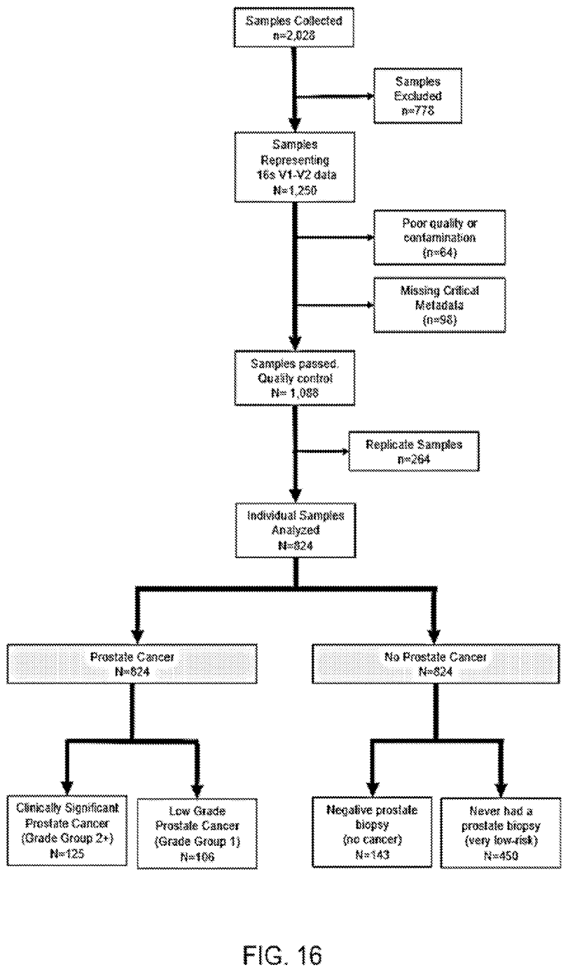

### DETAILED DESCRIPTION OF THE INVENTION

The present invention generally relates to a gut microbiome signature for cancers, including prostate cancer (PCa), using a prostate specific antigen (PSA)-independent, gut microbiome-based PCa biomarker, which can be used for diagnosis, suitability for screening, treatment decisions and as part of a treatment method.

A method is provided of determining whether a gut microbiome sample from a human subject is associated with a clinically significant cancer comprising:
(a) obtaining or having obtained a gut microbiome-containing sample from a
(b) performing a genetic analysis on the gut microbiome-containing sample so as to determine a (i) presence or not and (ii) abundance value of each of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) comparing the numerical microbiome score generated in (c) with a predetermined reference value which delineates a cutoff between (i) clinically significant cancer and (ii) low-risk cancer/negative biopsy, so as to determine if the gut microbiome sample is associated with a clinically significant cancer,
wherein a numerical microbiome score equal to or greater than the predetermined reference value indicates the sample comes from a human subject at risk of clinically significant cancer and a numerical microbiome score less than the predetermined reference value indicates the sample comes from a human subject with low-risk of cancer/negative biopsy.

A method is provided for detecting the presence or absence of a clinically significant cancer in a human subject, the method comprising:
(a) obtaining or having obtained a gut microbiome-containing sample from a human subject;
(b) performing a genetic analysis on the gut microbiome-containing sample, so as to determine a (i) presence or not and (ii) abundance value of each of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) detecting:
   i) the presence of a clinically significant cancer if the numerical microbiome score generated in (c) is equal to or greater than a predetermined reference value which delineates a cutoff between clinically significant cancer and low-risk cancer/negative biopsy; and
   ii) the absence of a clinically significant cancer, defined as low-risk of cancer/negative biopsy, if the numerical microbiome score generated in (c) is less than the predetermined reference value.

In embodiments, the numerical microbiome score generated in (c) is generated by:
(Y) for each bacterial taxon listed in (b):
   tabulating the bacterial taxon abundance value according to its read count and converting to a percentage abundance value; and
   providing the percentage abundance value to a random forest model, wherein each tree in the random forest model applies its own threshold(s) to produce a classification output;
   and
(Z) summing all classification outputs produced in (Y) from all the bacterial taxa so as to create a numerical microbiome score reflecting the majority vote for the sample.

In embodiments, the percentage abundance values are provided to a random forest model associated with determining a risk of a clinically significant cancer.

In embodiments, the percentage abundance values are provided to a random forest associated with determining a risk of a cancer.

In embodiments, the method further comprises determining whether a gut microbiome sample from a human subject is associated with a risk of a cancer and wherein a second numerical microbiome score is generated by:
(W) for each bacterial taxon listed in (b):
   tabulating the bacterial taxon abundance value according to its read count and converting to a percentage abundance value; and
   providing the percentage abundance value to a random model associated with determining a risk of cancer, wherein each tree in the random forest model applies its own threshold(s) to produce a classification output; and
(X) summing all classification outputs produced in (W) from all the bacterial taxa so as to create a second numerical microbiome score reflecting the majority vote for the sample,
wherein a second numerical microbiome score equal to or greater than a second predetermined reference value which delineates a cutoff between (i) a risk of a cancer and (ii) no current risk of cancer indicates the sample comes from a human subject at risk of a cancer and a second numerical microbiome score less than the second predetermined reference value indicates the sample comes from a human subject not currently at risk of cancer.

In embodiments, the method further comprises whether a gut microbiome sample from a human subject is associated with a risk of a clinically significant cancer and

wherein a numerical second numerical microbiome score is generated by:
(U) for each bacterial taxon listed in (b):
   tabulating the bacterial taxon abundance according to its read count and converting to a percentage abundance value; and
   providing the percentage abundance value to a random model associated with determining a clinically significant cancer, wherein each tree in the random forest model applies its own threshold(s) to produce a classification output;
   and
(V) summing all tree classification outputs produced in (U) from all the bacterial taxa so as to create a second numerical microbiome score reflecting the majority vote for the sample,
wherein a second numerical microbiome score equal to or greater than a second predetermined reference value which delineates a cutoff between (i) a clinically significant cancer and (ii) low-risk cancer/negative biopsy indicates the sample comes from a human subject at risk of a clinically significant cancer and a numerical microbiome score less than the second predetermined reference value indicates the sample comes from a human subject of low-risk cancer/negative biopsy.

In embodiments, the following taxa are the subject of the genetic analysis:
(a) k_Bacteria.p_Proteobacteria
(b) k_Bacteria.p_Actinobacteria.c_Actinobacteria
(c) k_Bacteria.p_Bacteroidetes.c_Bacteroidia
(d) k_Bacteria.p_Firmicutes.c_Bacilli
(e) k_Bacteria.p_Firmicutes.c_Erysipelotrichi
(f) k_Bacteria.p_Proteobacteria.c_Deltaproteobacteria
(g) k_Bacteria.p_Proteobacteria.c_Epsilonproteobacteria
(h) k_Bacteria.p_Proteobacteria.c_Gammaproteobacteria
(i) k_Bacteria.p_Actinobacteria.c_Actinobacteria.o_Actinomycetales
(i) k_Bacteria.p_Bacteroidetes.c_Bacteroidia.o_Bacteroidales
(k) k_Bacteria.p_Firmicutes.c_Bacilli.o_Lactobacilles
(l) k_Bacteria.p_Firmicutes.c_Erysipelotrichi.o_Erysipelotrichales
(m) k_Bacteria.p_Proteobacteria.c_Deltaproteobacteria.o_Desulfovibrionales
(n) k_Bacteria.p_Proteobacteria.c_Epsilonproteobacteria.o_Campylobacterales
(o) k_Bacteria.p_Proteobacteria.c_Gammaproteobacteria.o_Enterobacteriales
(p) k_Bacteria.p_Actinobacteria.c_Actinobacteria.o_Actinomycetales.f_Corynebacteriaceae
(q) k_Bacteria.p_Bacteroidetes.c_Bacteroidia.o_Bacteroidales.f_Bacteroidaceae
(r) k_Bacteria.p_Bacteroidetes.c_Bacteroidia.o_Bacteroidales.f_Odoribacteraceae(s) k_Bacteria.p_Bacteroidetes.c_Bacteroidia.o_Bacteroidales.f_Paraprevotellaceae
(t) k_Bacteria.p_Bacteroidetes.c_Bacteroidia.o_Bacteroidales.f_Porphyromonadaceae
(u) k_Bacteria.p_Bacteroidetes.c_Bacteroidia.o_Bacteroidales.f_Rikenellaceae
(v) k_Bacteria.p_Firmicutes.c_Clostridia.o_Clostridiales.f_Ruminococcaceae
(w) k_Bacteria.p_Firmicutes.c_Clostridia.o_Clostridiales.f_Tissierellaceae
(x) k_Bacteria.p_Firmicutes.c_Erysipelotrichi.o_Erysipelotrichales.f_Erysipelotrichaceae
(y) k_Bacteria.p_Proteobacteria.c_Deltaproteobacteria.o_Desulfovibrionales.f_Desulfovibrionaceae
(z) k_Bacteria.p_Proteobacteria.c_Epsilonproteobacteria.o_Campylobacterales.f_Campylobacteraceae
(aa) k_Bacteria.p_Proteobacteria.c_Gammaproteobacteria.o_Enterobacteriales.f_Enterobacteriaceae
(bb) k_Bacteria.p_Actinobacteria.c_Actinobacteria.o_Actinomycetales.f_Corynebacteriaceae.g_Corynebacterium
(cc) k_Bacteria.p_Bacteroidetes.c_Bacteroidia.o_Bacteroidales.f_Bacteroidaceae.g_Bacteroides
(dd) k_Bacteria.p_Firmicutes.c_Clostridia.o_Clostridiales.f_Lachnospiraceae.g_Coprococcus
(ee) k_Bacteria.p_Firmicutes.c_Clostridia.o_Clostridiales.f_Lachnospiraceae.g_Lachnospira
(ff) k_Bacteria.p_Firmicutes.c_Clostridia.o_Clostridiales.f_Ruminococcaceae.g_Oscillospira
(gg) k_Bacteria.p_Firmicutes.c_Clostridia.o_Clostridiales.f_Tissierellaceae.g_Finegoldia
(hh) k_Bacteria.p_Firmicutes.c_Clostridia.o_Clostridiales.f_Tissierellaceae.g_Peptoniphilus
(ii) k_Bacteria.p_Firmicutes.c_Clostridia.o_Clostridiales.f_Veillonellaceae.g_Dialister
(jj) k_Bacteria.p_Firmicutes.c_Erysipelotrichi.o_Erysipelotrichales.f_Erysipelotrichaceae.g_Eubacterium
(kk) k_Bacteria.p_Proteobacteria.c_Epsilonproteobacteria.o_Campylobacterales.f_Campylobacteraceae.g_Campylobacter,
(ll) k_Bacteria.p_Proteobacteria.c_Gammaproteobacteria.o_Pasteurellales.f_Pasteurellaceae.g_Haemophilus).

In embodiments, the cancer is prostate cancer.

In embodiments, the clinically significant cancer is Grade Group 2 (GG2) or greater and the low-risk cancer/negative biopsy is Grade Group 1 (GG1) or below.

In embodiments, the cancer is colon cancer or lung cancer.

In embodiments, the sample is a fecal sample.

In embodiments, the sample is a fecal sample obtained by a swab.

In embodiments, the sample has been obtained by the subject.

In embodiments, the genetic analysis is performed for a variable region of bacterial 16S rRNA genes.

In embodiments, the genetic analysis comprises one or more of paired end read trimming and merging; primer removal; QIIME; QC filtering; chimera detection; human DNA detection; and non-16S rRNA gene detection, so as to produce a set of 16S rRNA gene sequences for analysis.

In embodiments, the method further comprises filtering out from the analysis genetic analysis, presence indication, and/or abundance values of the following background taxa prior to calculating the numerical microbiome score:
(a) c_Chloroplast
(b) c_Alphaproteobacteria
(c) f Propionibacteriaceae
(d) o_Oceanospirillales
(e) f_Intrasporangiaceae
(f) f_Micrococcaceae
(g) f_Cytophagaceae
(h) o_Hydrogenophilales
(i) f_Alcaligenaceae
(j) f Burkholderiaceae
(k) f Burkholderiales_incertae_sedis
(l) f Comamonadaceae
(m) f Oxalobacteraceae.

In embodiments, the genetic analysis is performed on variable regions 1 and 2 and/or variable regions 3 and 4 of the 16S rRNA gene of each taxon.

In embodiments, the numerical microbiome score generated in (c) is generated by, for each bacterial taxon listed in (b):
i) ascribing a 1 or a 0 if present or not present in the sample, respectively, so as to generate a presence value;
ii) ascribing a predetermined weighting value to each bacterial taxon, so as to generate a weighted bacteria taxon value;
iii) modifying the abundance value determined by a predetermined weighting for each bacterial taxon, so as to generate a weighted abundance value; and
iv) inputting the presence value, weighted abundance value, and weighted bacterial taxon value into a formula and applying the formula so as to generate an output which is the numerical microbiome score.

In embodiments, the methods further comprise treating a subject, identified as having a clinically significant prostate cancer by the method, comprising administering an androgen suppression therapy, abiraterone, external beam radiation (with or without brachytherapy), and/or a radical prostatectomy (with or without pelvic lymph node dissection).

A method is provided of determining whether a gut microbiome sample from a human subject is associated with a cancer risk comprising:
(a) obtaining or having obtained a gut microbiome-containing sample from a
(b) performing a genetic analysis on the gut microbiome-containing sample so as to determine a (i) presence or not and (ii) abundance value of each of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) comparing the numerical microbiome score generated in (c) with a predetermined reference value which delineates a cutoff between (i) a cancer risk and (ii) no current risk of cancer, so as to determine if the gut microbiome sample is associated with a cancer risk,
wherein a numerical microbiome score equal to or greater than the predetermined reference value indicates the sample comes from a human subject at risk of a cancer and a numerical microbiome score less than the predetermined reference value indicates the sample comes from a human subject not currently at risk of cancer.

A method is provided of determining whether a gut microbiome sample from a human subject is associated with a cancer risk comprising:
(a) obtaining or having obtained a gut microbiome-containing sample from a
(b) performing a genetic analysis on the gut microbiome-containing sample so as to determine a (i) presence or not and (ii) abundance value of each of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) determining:
   i) that the gut microbiome sample from the human subject is associated with a current cancer risk if the numerical microbiome score generated in (c) is equal to or greater than a predetermined reference value which delineates a cutoff between a cancer risk and no current risk of cancer;
   ii) that the gut microbiome sample from the human subject is not associated with a current cancer risk if the numerical microbiome score generated in (c) is less than the predetermined reference value.

A method is provided of identifying a subject as in need of a cancer screening test, comprising:
(a) obtaining or having obtained a gut microbiome-containing sample from a human subject;
(b) performing a genetic analysis on the gut microbiome-containing sample so as to determine a (i) presence or not and (ii) abundance value of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) comparing the numerical microbiome score generated in (c) with a predetermined reference value which delineates a cutoff between (i) clinically significant cancer and (ii) low-risk cancer/negative biopsy, so as to determine if the gut microbiome sample is associated with a clinically significant cancer,
wherein a numerical microbiome score equal to or greater than the cutoff indicates the sample comes from a human subject in need of a cancer screening test and a numerical microbiome score less than the cutoff indicates the sample comes from a human subject not in need of a cancer screening test.

A method is provided of identifying a subject as in need or not in need of a cancer screening test, comprising:
(a) obtaining or having obtained a gut microbiome-containing sample from a
(b) performing a genetic analysis on the gut microbiome-containing sample so as to determine a (i) presence or not and (ii) abundance value of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) identifying the subject as:
   i) in need of a cancer screening test if the numerical microbiome score generated in (c) is equal to or greater than a predetermined reference value which delineates a cutoff between clinically significant cancer and low-risk cancer/negative biopsy;
   ii) not in need of a cancer screening test if the numerical microbiome score generated in (c) is less than the predetermined reference value.

In embodiments, the following taxa are the subject of the genetic analysis:
(a) k_Bacteria.p_Proteobacteria
(b) k_Bacteria.p_Actinobacteria.c_Actinobacteria
(c) k_Bacteria.p_Bacteroidetes.c_Bacteroidia
(d) k_Bacteria.p_Firmicutes.c_Bacilli
(e) k_Bacteria.p_Firmicutes.c_Erysipelotrichi
(f) k_Bacteria.p_Proteobacteria.c_Deltaproteobacteria
(g) k_Bacteria.p_Proteobacteria.c_Epsilonproteobacteria
(h) k_Bacteria.p_Proteobacteria.c_Gammaproteobacteria
(i) k_Bacteria.p_Actinobacteria.c_Actinobacteria.o_Actinomycetales
(i) k_Bacteria.p_Bacteroidetes.c_Bacteroidia.o_Bacteroidales
(k) k_Bacteria.p_Firmicutes.c_Bacilli.o_Lactobacillus
(l) k_Bacteria.p_Firmicutes.c_Erysipelotrichi.o_Erysipelotrichales
(m) k_Bacteria.p_Proteobacteria.c_Deltaproteobacteria.o_Desulfovibrionales
(n) k_Bacteria.p_Proteobacteria.c_Epsilonproteobacteria.o_Campylobacterales
(o) k_Bacteria.p_Proteobacteria.c_Gammaproteobacteria.o_Enterobacteriales
(p) k_Bacteria.p_Actinobacteria.c_Actinobacteria.o_Actinomycetales.f_Corynebacteriaceae
(q) k_Bacteria.p_Bacteroidetes.c_Bacteroidia.o_Bacteroidales.f_Bacteroidaceae
(r) k_Bacteria.p_Bacteroidetes.c_Bacteroidia.o_Bacteroidales.f_Odoribacteraceae(s)
(s) k_Bacteria.p_Bacteroidetes.c_Bacteroidia.o_Bacteroidales.f_Paraprevotellaceae
(t) k_Bacteria.p_Bacteroidetes.c_Bacteroidia.o_Bacteroidales.f_Porphyromonadaceae
(u) k_Bacteria.p_Bacteroidetes.c_Bacteroidia.o_Bacteroidales.f_Rikenellaceae
(v) k_Bacteria.p_Firmicutes.c_Clostridia.o_Clostridiales.f_Ruminococcaceae
(w) k_Bacteria.p_Firmicutes.c_Clostridia.o_Clostridiales.f_Tissierellaceae
(x) k_Bacteria.p_Firmicutes.c_Erysipelotrichi.o_Erysipelotrichales.f_Erysipelotrichaceae
(y) k_Bacteria.p_Proteobacteria.c_Deltaproteobacteria.o_Desulfovibrionales.f_Desulfovibrionaceae
(z) k_Bacteria.p_Proteobacteria.c_Epsilonproteobacteria.o_Campylobacterales.f_Campylobacteraceae
(aa) k_Bacteria.p_Proteobacteria.c_Gammaproteobacteria.o_Enterobacteriales.f_Enterobacteriaceae
(bb) k_Bacteria.p_Actinobacteria.c_Actinobacteria.o_Actinomycetales.f_Corynebacteriaceae.g_Corynebacterium
(cc) k_Bacteria.p_Bacteroidetes.c_Bacteroidia.o_Bacteroidales.f_Bacteroidaceae.g_Bacteroides
(dd) k_Bacteria.p_Firmicutes.c_Clostridia.o_Clostridiales.f_Lachnospiraceae.g_Coprococcus
(ee) k_Bacteria.p_Firmicutes.c_Clostridia.o_Clostridiales.f_Lachnospiraceae.g_Lachnospira
(ff) k_Bacteria.p_Firmicutes.c_Clostridia.o_Clostridiales.f_Ruminococcaceae.g_Oscillospira
(gg) k_Bacteria.p_Firmicutes.c_Clostridia.o_Clostridiales.f_Tissierellaceae.g_Finegoldia
(hh) k_Bacteria.p_Firmicutes.c_Clostridia.o_Clostridiales.f_Tissierellaceae.g_Peptoniphilus
(ii) k_Bacteria.p_Firmicutes.c_Clostridia.o_Clostridiales.f_Veillonellaceae.g_Dialister
(jj) k_Bacteria.p_Firmicutes.c_Erysipelotrichi.o_Erysipelotrichales.f_Erysipelotrichaceae.g_Eubacterium
(kk) k_Bacteria.p_Proteobacteria.c_Epsilonproteobacteria.o_Campylobacterales.f_Campylobacteraceae.g_Campylobacter,
(ll) k_Bacteria.p_Proteobacteria.c_Gammaproteobacteria.o_Pasteurellales.f_Pasteurellaceae.g_Haemophilus.

In embodiments, the methods further comprise treating a subject, identified as having a clinically significant prostate cancer by the method, comprising administering an androgen suppression therapy, abiraterone, external beam radiation (with or without brachytherapy), and/or a radical prostatectomy (with or without pelvic lymph node dissection).

A method is provided of treating a prostate cancer in a subject comprising:
(A) obtaining, or having obtained, a diagnosis of the subject as having a clinically significant prostate cancer by a method comprising, on a gut microbiome-containing sample from a human subject,
   a) performing a genetic analysis on the sample so as to determine a (i) presence or not and (ii) abundance value of each of the following taxa:
      g_Oscillospora;
      g_Lachnospira;
      f_Corynebacteriaceae;
      g_Corynebacterium;
      o_Actinomycetales;
      f_Paraprevotellaceae;
      g_Eubacterium;
      g_Peptoniphilus;
      o_Enterobacteriales;
      c_Erysipelotrichi;
      c_Gammaproteobacteria;
      g_Finegoldia;
      p_Proteobacteria;
      f Bacteroidaceae; and
      c_Deltaproteobacteria;
   b) generating a numerical microbiome score from the genetic analysis performed in a),
   c) comparing the numerical microbiome score generated in b) with a predetermined reference value which delineates a cutoff between (i) clinically significant cancer and (ii) low-risk cancer/negative biopsy, so as to determine if the gut microbiome sample is associated with a clinically significant cancer,
   wherein a numerical microbiome score equal to or greater than the predetermined reference value indicates the sample comes from a human subject at risk of a clinically significant cancer and a numerical microbiome score less than the predetermined reference value indicates the sample comes from a human subject with low-risk of cancer/negative biopsy,
(B) treating a subject, having a diagnosis in (A) as having a clinically significant prostate cancer, comprising administering an androgen suppression therapy, abiraterone, external beam radiation (with or without brachytherapy), and/or radical prostatectomy (with or without pelvic lymph node dissection) to the subject.

A method of treating a prostate cancer in a subject comprising:
(A) obtaining, or having obtained, a diagnosis of the subject as having a clinically significant prostate cancer by a method comprising, on a gut microbiome-containing sample from a human subject,
   a) performing a genetic analysis on the sample so as to determine a (i) presence or not and (ii) abundance value of each of the following taxa:
      g_Oscillospora,
      g_Lachnospira;
      f_Corynebacteriaceae;
      g_Corynebacterium;
      o_Actinomycetales;
      f_Paraprevotellaceae;
      g_Eubacterium;
      g_Peptoniphilus;
      o_Enterobacteriales;
      c_Erysipelotrichi;
      c_Gammaproteobacteria;
      g_Finegoldia;
      p_Proteobacteria;
      f_Bacteroidaceae; and
      c_Deltaproteobacteria;
   b) generating a numerical microbiome score from the genetic analysis performed in a),
   c) diagnosing the subject as:
      (i) having a clinically significant prostate cancer if the numerical microbiome score generated in b) is equal to or greater than a predetermined reference value which delineates a cutoff between clinically significant cancer and low-risk cancer/negative biopsy,
      (ii) not having a clinically significant prostate cancer, defined as low-risk cancer/negative biopsy, if the numerical microbiome score generated in b) is less than the predetermined reference value,
(B) treating the subject, having a diagnosis in (A) as having a clinically significant prostate cancer, comprising administering an androgen suppression therapy, abiraterone, external beam radiation (with or without brachytherapy), and/or radical prostatectomy (with or without pelvic lymph node dissection) to the subject.

In embodiments, the numerical microbiome score generated in b) is generated by:
(Y) for each bacterial taxon listed in a):
   tabulating the bacterial taxon abundance value according to its read count and converting to a percentage abundance value; and
   providing the percentage abundance value to a random forest model, wherein each tree in the random forest model applies its own threshold(s) to produce a classification output;
   and
(Z) summing all classification outputs produced in (Y) from all the bacterial taxa so as to create a numerical microbiome score reflecting the majority vote for the sample.

In embodiments, the percentage abundance values are provided to a random forest model associated with determining a clinically significant cancer.

In embodiments, the numerical microbiome score is from 0 to 1 and the predetermined reference value for risk of a clinically significant cancer is 0.158.

In embodiments, the numerical microbiome score is from 0 to 1 and the predetermined reference value for risk of a cancer is 0.25 or above.

In embodiments, the numerical microbiome score is from 0 to 1 and the predetermined reference value for risk of a cancer is 0.256.

In embodiments, the predetermined refence value associates with a prostate cancer or prostate cancer risk is determined as set forth below under “Microbiome Signature identification”.

In embodiments, the following taxa are the subject of the genetic analysis:
(a) k_Bacteria.p_Proteobacteria
(b) k_Bacteria.p_Actinobacteria.c_Actinobacteria
(c) k_Bacteria.p_Bacteroidetes.c_Bacteroidia
(d) k_Bacteria.p_Firmicutes.c_Bacilli
(e) k_Bacteria.p_Firmicutes.c_Erysipelotrichi
(f) k_Bacteria.p_Proteobacteria.c_Deltaproteobacteria
(g) k_Bacteria.p_Proteobacteria.c_Epsilonproteobacteria
(h) k_Bacteria.p_Proteobacteria.c_Gammaproteobacteria
(i) k_Bacteria.p_Actinobacteria.c_Actinobacteria.o_Actinomycetales
(i) k_Bacteria.p_Bacteroidetes.c_Bacteroidia.o_Bacteroidales
(k) k_Bacteria.p_Firmicutes.c_Bacilli.o_Lactobacilles
(l) k_Bacteria.p_Firmicutes.c_Erysipelotrichi.o_Erysipelotrichales
(m) k_Bacteria.p_Proteobacteria.c_Deltaproteobacteria.o_Desulfovibrionales
(n) k_Bacteria.p_Proteobacteria.c_Epsilonproteobacteria.o_Campylobacterales
(o) k_Bacteria.p_Proteobacteria.c_Gammaproteobacteria.o_Enterobacteriales
(p) k_Bacteria.p_Actinobacteria.c_Actinobacteria.o_Actinomycetales.f_Corynebacteriaceae
(q) k_Bacteria.p_Bacteroidetes.c_Bacteroidia.o_Bacteroidales.f_Bacteroidaceae
(r) k_Bacteria.p_Bacteroidetes.c_Bacteroidia.o_Bacteroidales.f_Odoribacteraceae(s)
k_Bacteria.p_Bacteroidetes.c_Bacteroidia.o_Bacteroidales.f_Paraprevotellaceae
(t) k_Bacteria.p_Bacteroidetes.c_Bacteroidia.o_Bacteroidales.f_Porphyromonadaceae
(u) k_Bacteria.p_Bacteroidetes.c_Bacteroidia.o_Bacteroidales.f_Rikenellaceae
(v) k_Bacteria.p_Firmicutes.c_Clostridia.o_Clostridiales.f_Ruminococcaceae
(w) k_Bacteria.p_Firmicutes.c_Clostridia.o_Clostridiales.f_Tissierellaceae
(x) k_Bacteria.p_Firmicutes.c_Erysipelotrichi.o_Erysipelotrichales.f_Erysipelotrichaceae
(y) k_Bacteria.p_Proteobacteria.c_Deltaproteobacteria.o_Desulfovibrionales.f_Desulfovibrionaceae
(z) k_Bacteria.p_Proteobacteria.c_Epsilonproteobacteria.o_Campylobacterales.f_Campylobacteraceae
(aa) k_Bacteria.p_Proteobacteria.c_Gammaproteobacteria.o_Enterobacteriales.f_Enterobacteriaceae
(bb) k_Bacteria.p_Actinobacteria.c_Actinobacteria.o_Actinomycetales.f_Corynebacteriaceae.g_Corynebacterium (cc) k_Bacteria.p_Bacteroidetes.c_Bacteroidia.o_Bacteroidales.f_Bacteroidaceae.g_Bacteroides
(dd) k_Bacteria.p_Firmicutes.c_Clostridia.o_Clostridiales.f_Lachnospiraceae.g_Coprococcus
(ee) k_Bacteria.p_Firmicutes.c_Clostridia.o_Clostridiales.f_Lachnospiraceae.g_Lachnospira
(ff) k_Bacteria.p_Firmicutes.c_Clostridia.o_Clostridiales.f_Ruminococcaceae.g_Oscillospira
(gg) k_Bacteria.p_Firmicutes.c_Clostridia.o_Clostridiales.f_Tissierellaceae.g_Finegoldia
(hh) k_Bacteria.p_Firmicutes.c_Clostridia.o_Clostridiales.f_Tissierellaceae.g_Peptoniphilus
(ii) k_Bacteria.p_Firmicutes.c_Clostridia.o_Clostridiales.f_Veillonellaceae.g Dialister
(jj) k_Bacteria.p_Firmicutes.c_Erysipelotrichi.o_Erysipelotrichales.f_Erysipelotrichaceae.g_Eubacterium (kk) k_Bacteria.p_Proteobacteria.c_Epsilonproteobacteria.o_Campylobacterales.f_Campylobacteraceae.g_Campylobacter,
(ll) k_Bacteria.p_Proteobacteria.c_Gammaproteobacteria.o_Pasteurellales.f_Pasteurellaceae.g_Haemophilus.

In embodiments, the methods further comprise performing the method on the first individual subject at one or more timepoints subsequent to an initial performance of the method on the first individual subject when the subject has been identified by the method as having risk of clinically significant cancer by the initial performance, and comparing the numerical microbiome score for the initial and subsequent one or more timepoint performances so as to determine if risk of clinically significant cancer identified in the subject in initial performance is (a) worsening, (b) improving, or (c) showing no change.

In embodiments, the first individual subject is administered a clinical cancer treatment subsequent to the initial performance of the method on the first individual subject and prior to at least one of the one or more timepoints subsequent.

In embodiments of the methods, the percentage abundance values are provided to a random forest model associated with determining a clinically significant cancer.

In embodiments, the random forest plot, which may be a machine learning model, may be trained by randomly generating decision trees. Each node in a decision tree of the random forest may be configured to consider a random subset of features, which may be various taxa, to calculate an output of the decision tree. The random forest may then combine the output of individual decision trees to generate a final output. In embodiments, bootstrapping, which is the process of randomly selecting items from a training dataset, may also be employed. In embodiments, bootstrapping may assemble randomized decisions based on several decisions and makes the final decision based on the majority voting.

In embodiments of the methods, the numerical microbiome score is from 0 to 1 and the predetermined reference value for risk of a clinically significant cancer is 0.158.

In embodiments of the methods, the numerical microbiome score is from 0 to 1 and the predetermined reference value for risk of a cancer is 0.256.

A kit is provided comprising (a) primer pairs and (b) one or more reagents, wherein in (a) the primer pairs consists essentially of primer pairs for all of:
g_Oscillospora;
g_Lachnospira;
f_Corynebacteriaceae;
g_Corynebacterium;
o_Actinomycetales;
f_Paraprevotellaceae;
g_Eubacterium;
g Peptoniphilus;
o_Enterobacteriales;
c_Erysipelotrichi;
c_Gammaproteobacteria;
g_Finegoldia;
p_Proteobacteria;
f Bacteroidaceae; and
c_Deltaproteobacteria.

A method is provided for determining cancer risk using a computer system comprising one or more processors operably connected to one or more memories, the method comprising:
(a) accessing, by the one or more processors, from the one or more memories, digitized sequence data associated with a gut microbiome-containing sample;
(b) generating, by the one or more processors, 16S rRNA sequence data based on the digitized sequence data;
(c) generating, by the one or more processors, a normalized matrix representing (i) presence or not and (ii) abundance of taxa associated with the 16S rRNA sequence data;
(d) selecting, by the one or more processors, prevalence data in the normalized matrix which is associated with specific taxonomic features;
(e) (e) obtaining, by an artificial intelligence module of the computer system comprising at least one artificial intelligence model, the prevalence data;
(f) generating an output, by the artificial intelligence module comprising the at least one artificial intelligence model trained using a training set, based on a query wherein:
   i) the query comprises the prevalence data;
   ii) the output comprises a numerical microbiome score;
   iii) the training set comprises data representing the prevalence of the specific taxonomic features and associated cancer development in patients;
In embodiments, the artificial intelligence module may be a machine learning module. In embodiments, the at least one artificial intelligence model may be a machine learning model. For example, the at least one artificial intelligence model may utilize a model including or approximating a random forest model.

In embodiments, the specific taxonomic features may include:
(a) k_Bacteria.p_Proteobacteria
(b) k_Bacteria.p_Actinobacteria.c_Actinobacteria
(c) k_Bacteria.p_Bacteroidetes.c_Bacteroidia
(d) k_Bacteria.p_Firmicutes.c_Bacilli
(e) k_Bacteria.p_Firmicutes.c_Erysipelotrichi
(f) k_Bacteria.p_Proteobacteria.c_Deltaproteobacteria
(g) k_Bacteria.p_Proteobacteria.c_Epsilonproteobacteria
(h) k_Bacteria.p_Proteobacteria.c_Gammaproteobacteria
(i) k_Bacteria.p_Actinobacteria.c_Actinobacteria.o_Actinomycetales
(i) k_Bacteria.p_Bacteroidetes.c_Bacteroidia.o_Bacteroidales
(k) k_Bacteria.p_Firmicutes.c_Bacilli.o_Lactobacillus
(l) k_Bacteria.p_Firmicutes.c_Erysipelotrichi.o_Erysipelotrichales
(m) k_Bacteria.p_Proteobacteria.c_Deltaproteobacteria.o_Desulfovibrionales
(n) k_Bacteria.p_Proteobacteria.c_Epsilonproteobacteria.o_Campylobacterales
(o) k_Bacteria.p_Proteobacteria.c_Gammaproteobacteria.o_Enterobacteriales
(p) k_Bacteria.p_Actinobacteria.c_Actinobacteria.o_Actinomycetales.f_Corynebacteriaceae
(q) k_Bacteria.p_Bacteroidetes.c_Bacteroidia.o_Bacteroidales.f_Bacteroidaceae
(r) k_Bacteria.p_Bacteroidetes.c_Bacteroidia.o_Bacteroidales.f_Odoribacteraceae(s)
k_Bacteria.p_Bacteroidetes.c_Bacteroidia.o_Bacteroidales.f_Paraprevotellaceae
(t) k_Bacteria.p_Bacteroidetes.c_Bacteroidia.o_Bacteroidales.f_Porphyromonadaceae
(u) k_Bacteria.p_Bacteroidetes.c_Bacteroidia.o_Bacteroidales.f_Rikenellaceae
(v) k_Bacteria.p_Firmicutes.c_Clostridia.o_Clostridiales.f_Ruminococcaceae
(w) k_Bacteria.p_Firmicutes.c_Clostridia.o_Clostridiales.f_Tissierellaceae
(x) k_Bacteria.p_Firmicutes.c_Erysipelotrichi.o_Erysipelotrichales.f_Erysipelotrichaceae
(y) k_Bacteria.p_Proteobacteria.c_Deltaproteobacteria.o_Desulfovibrionales.f_Desulfovibrionaceae
(z) k_Bacteria.p_Proteobacteria.c_Epsilonproteobacteria.o_Campylobacterales.f_Campylobacteraceae
(aa) k_Bacteria.p_Proteobacteria.c_Gammaproteobacteria.o_Enterobacteriales.f_Enterobacteriaceae
(bb) k_Bacteria.p_Actinobacteria.c_Actinobacteria.o_Actinomycetales.f_Corynebacteriaceae.g_Corynebacterium
(cc) k_Bacteria.p_Bacteroidetes.c_Bacteroidia.o_Bacteroidales.f_Bacteroidaceae.g_Bacteroides
(dd) k_Bacteria.p_Firmicutes.c_Clostridia.o_Clostridiales.f_Lachnospiraceae.g_Coprococcus
(ee) k_Bacteria.p_Firmicutes.c_Clostridia.o_Clostridiales.f_Lachnospiraceae.g_Lachnospira
(ff) k_Bacteria.p_Firmicutes.c_Clostridia.o_Clostridiales.f_Ruminococcaceae.g_Oscillospira
(gg) k_Bacteria.p_Firmicutes.c_Clostridia.o_Clostridiales.f_Tissierellaceae.g_Finegoldia
(hh) k_Bacteria.p_Firmicutes.c_Clostridia.o_Clostridiales.f_Tissierellaceae.g_Peptoniphilus
(ii) k_Bacteria.p_Firmicutes.c_Clostridia.o_Clostridiales.f_Veillonellaceae.g_Dialister
(jj) k_Bacteria.p_Firmicutes.c_Erysipelotrichi.o_Erysipelotrichales.f_Erysipelotrichaceae.g_Eubacterium
(kk) k_Bacteria.p_Proteobacteria.c_Epsilonproteobacteria.o_Campylobacterales.f_Campylobacteraceae.g_Campylobacter,
(ll) k_Bacteria.p_Proteobacteria.c_Gammaproteobacteria.o_Pasteurellales.f_Pasteurellaceae.g_Haemophilus.

In embodiments, the specific taxonomic features are hard coded. In embodiments, the specific taxonomic features are stored in data as a table.

In embodiments, the generating the 16S rRNA sequence data comprises removing artifacts from the digitized sequence data.

In embodiments, removing artifacts from the digitized sequence data comprises one or more of the steps of:
(1) trimming data associated with paired sequences,
(2) denoising the data associated with the paired sequences,
(3) merging the data associated with the paired sequences
(4) removing data representing RNA primers;
(5) performing filtering on the digitized sequence data;
(6) detecting and filtering data associated with chimeras;
(7) detecting and filtering data associated with non-Human rRNA sequences;
(8) detecting and filtering data associated with non-16S rRNA sequences.

In embodiments, the method further comprises storing the 16S rRNA sequence data in the one or more memories. For example, a function call may be used to save the 16S rRNA sequence data in csv format.

In embodiments, generating the normalized matrix comprises:
(1) generating a taxonomic matrix, by the steps of:
   i. generating clusters of similar sequences represented in the 16S rRNA sequence data, wherein the clustering is based on a similarity threshold;
   ii. assigning one or more taxonomies to the clusters; and
   iii. assigning counts to each of the one or more taxonomies based on the 16S rRNA data.
(2) generating a filtered matrix by removing data from the taxonomic matrix which associated with one or more contaminant taxa;
(3) conducting rarefaction based on a specified sample size;
(4) assigning data to the normalized matrix based on the rarefaction.

In embodiments, the clusters represent operational taxonomic units.

In embodiments, the similarity threshold is defined by a user.

In embodiments, the one or more contaminant taxa are selected from a group consisting of:
c_Chloroplast;
c_Alphaproteobacteria;
f_Propionibacteriaceae;
o_Oceanospirillales;
f_Intrasporangiaceae;
f_Micrococcaceae;
f_Cytophagaceae;
o_Hydrogenophilales;
f_Alcaligenaceae;
f_Burkholderiaceae;
f_Burkholderiales_incertae_sedis;
f Comamonadaceae; and
f_Oxalobacteraceae.

In embodiments, the one or more contaminant taxa are hard coded. In embodiments, the one or more contaminant taxa are stored in the one or more memories as a table.

In embodiments, the data are assigned at the genus level.

In embodiments, the prevalence data is modified to include data related to bacterial abundance and relative importance.

In embodiments, the at least one artificial intelligence model may be a machine learning model. In embodiments, the at least one artificial intelligence model comprises a random forest model. In embodiments, the at least one artificial intelligence model is trained using actual patient data. In embodiments, the at least one artificial intelligence model is trained using simulated patient data. In embodiments, the at least one artificial intelligence model comprises two artificial intelligence models and wherein the output comprises two distinct numerical microbiome scores. The two distinct numerical microbiome scores may represent scores associated two different grades of cancer. In embodiments, the at least one artificial intelligence model may comprise a greater number of artificial intelligence models than two.

In embodiments, the random forest plot, which may be a machine learning model, may be trained by randomly generating decision trees. Each node in a decision tree of the random forest may be configured to consider a random subset of features, which may be various taxa, to calculate an output of the decision tree. The random forest may then combine the output of individual decision trees to generate a final output. In embodiments, bootstrapping, which is the process of randomly selecting items from a training dataset, may also be employed. In embodiments, bootstrapping may assemble randomized decisions based on several decisions and makes the final decision based on the majority voting.

In embodiments, the numerical prevalence score comprises at least one of a cancer score and a Grade Group 2 (GG2) prostate cancer score.

In embodiments, the method further comprises storing the taxonomic matrix in the one or more memories. In embodiments, the method further comprises storing the filtered matrix in the one or more memories. In embodiments, the method further comprises storing the normalized matrix in the one or more memories.

In embodiments, the method further comprises displaying, by the one or more processors, elements of the taxonomic matrix in a graphical format. In embodiments, the method further comprises displaying, by the one or more processors, elements of the filtered matrix in a graphical format. In embodiments, the method further comprises displaying, by the one or more processors, elements of the normalized matrix in a graphical format.

In embodiments, the method further comprises displaying, by the one or more processors, the relative abundance of the specific taxonomic features.

In embodiments, the one or more processors may be operably connected to a display device (e.g., a monitor) capable of displaying elements of the taxonomic, filtered and/or normalized matrices, in a graphical format and/or the relative abundance of the specific taxonomic features. In embodiments, displaying elements of the taxonomic, filtered and/or normalized matrices and/or the relative abundance of the specific taxonomic features may comprise transmitting to a database to later be displayed upon request by a user.

In embodiments, the numerical microbiome score is a real number between zero and one and the estimated risk of cancer is calculated by the steps of:
i) multiplying the cancer score by 4.476;
ii) subtracting 4.421 from the result of the multiplying step; and
iii) calculating the exponent of Euler's Number to the power of the result of the subtracting step.

In embodiments, the method further comprises calculating a high risk of cancer estimate and a low risk of cancer estimate. In embodiments, the high risk of cancer estimate is half of the estimated risk of cancer. In embodiments, the low risk of cancer estimate is half of the estimated risk of cancer. In embodiments, the high and/or low risk of cancer estimates by the output of the at least one artificial intelligence model.

Also provided is a method for determining whether a gut microbiome sample from a human subject is associated with a risk of a future prostate cancer or risk of a future clinically significant prostate cancer comprising:
(a) obtaining, or having obtained, a gut microbiome-containing sample from a human subject;
(b) performing a genetic analysis on the gut microbiome-containing sample so as to determine a (i) presence or not and (ii) abundance value of each of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) comparing the numerical microbiome score generated in (c) with a predetermined reference value which delineates a cutoff between (i) risk of future prostate cancer or at risk of a future clinically significant prostate cancer and (ii) low-risk of future prostate cancer/negative biopsy, so as to determine if the gut microbiome sample is associated with a risk of future prostate cancer or risk of a future clinically significant prostate cancer,
wherein a numerical microbiome score equal to or greater than the predetermined reference value indicates the sample comes from a human subject at risk of future prostate cancer or risk of a future clinically significant prostate cancer, and a numerical microbiome score less than the predetermined reference value indicates the sample comes from a human subject with low-risk of future prostate cancer/negative biopsy.

Also provided is a method for determining whether bacteria of a gut microbiome sample from a human subject are associated with a risk of a future prostate cancer or risk of a future clinically significant prostate cancer comprising:
(a) obtaining, or having obtained, a gut microbiome-containing sample from a human subject;
(b) performing a genetic analysis on the gut microbiome-containing sample so as to determine a (i) presence or not and (ii) abundance value of each of the following taxa:
   g_Oscillospora;
   g_Lachnospira;
   f_Corynebacteriaceae;
   g_Corynebacterium;
   o_Actinomycetales;
   f_Paraprevotellaceae;
   g_Eubacterium;
   g_Peptoniphilus;
   o_Enterobacteriales;
   c_Erysipelotrichi;
   c_Gammaproteobacteria;
   g_Finegoldia;
   p_Proteobacteria;
   f_Bacteroidaceae; and
   c_Deltaproteobacteria;
(c) generating a numerical microbiome score from the genetic analysis performed in (b);
(d) determining:
   i) that the bacteria of the gut microbiome sample from the human subject are associated with a risk of a future prostate cancer or risk of a future clinically significant prostate cancer if the numerical microbiome score generated in (c) is equal to or greater than a predetermined reference value which delineates a cutoff between risk of future prostate cancer or at risk of a future clinically significant prostate cancer and low-risk of future prostate cancer/negative biopsy
   ii) that the bacteria of the gut microbiome sample from the human subject are not associated with a risk of a future prostate cancer or risk of a future clinically significant prostate cancer if the numerical microbiome score generated in (c) is less than a predetermined reference value.

In embodiments, the specific taxonomic features in determining a (i) presence or not and (ii) abundance value of each of the taxa comprise:
k_Bacteria.p_Bacteroidetes;
k_Bacteria.p_Proteobacteria;
k_Bacteria.p_Actinobacteria.c_Actinobacteria;
k_Bacteria.p_Bacteroidetes.c_Bacteroidia;
k_Bacteria.p_Firmicutes.c_Bacilli;
k_Bacteria.p_Firmicutes.c_Erysipelotrichi;
k_Bacteria.p_Proteobacteria.c_Deltaproteobacteria;
k_Bacteria.p_Proteobacteria.c_Epsilonproteobacteria;
k_Bacteria.p_Proteobacteria.c_Gammaproteobacteria;
k_Bacteria.p_Actinobacteria.c_Actinobacteria.o_Actinomycetales;
k_Bacteria.p_Bacteroidetes.c_Bacteroidia.o_Bacteroidales;
k_Bacteria.p_Firmicutes.c_Bacilli.o_Lactobacillus;
k_Bacteria.p_Firmicutes.c_Erysipelotrichi.o_Erysipelotrichales;
k_Bacteria.p_Proteobacteria.c_Deltaproteobacteria.o_Desulfovibrionales;
k_Bacteria.p_Proteobacteria.c_Epsilonproteobacteria.o_Campylobacterales;
k_Bacteria.p_Proteobacteria.c_Gammaproteobacteria.o_Enterobacteriales;
k_Bacteria.p_Actinobacteria.c_Actinobacteria.o_Actinomycetales.f_Corynebacteriaceae;
k_Bacteria.p_Bacteroidetes.c_Bacteroidia.o_Bacteroidales.f_Bacteroidaceae;
k_Bacteria.p_Bacteroidetes.c_Bacteroidia.o_Bacteroidales.f_Odoribacteraceae;
k_Bacteria.p_Bacteroidetes.c_Bacteroidia.o_Bacteroidales.f_Paraprevotellaceae;
k_Bacteria.p_Bacteroidetes.c_Bacteroidia.o_Bacteroidales.f_Porphyromonadaceae;
k_Bacteria.p_Bacteroidetes.c_Bacteroidia.o_Bacteroidales.f_Rikenellaceae;
k_Bacteria.p_Firmicutes.c_Clostridia.o_Clostridiales.f_Ruminococcaceae;
k_Bacteria.p_Firmicutes.c_Clostridia.o_Clostridiales.f_Tissierellaceae;
k_Bacteria.p_Firmicutes.c_Erysipelotrichi.o_Erysipelotrichales.f_Erysipelotrichaceae;
k_Bacteria.p_Proteobacteria.c_Deltaproteobacteria.o_Desulfovibrionales.f_Desulfovibrionaceae;
k_Bacteria.p_Proteobacteria.c_Epsilonproteobacteria.o_Campylobacterales.f_Campylobacteraceae;
k_Bacteria.p_Proteobacteria.c_Gammaproteobacteria.o_Enterobacteriales.f_Enterobacteriaceae;
k_Bacteria.p_Actinobacteria.c_Actinobacteria.o_Actinomycetales.f_Corynebacteriaceae.g_Corynebacterium;
k_Bacteria.p_Bacteroidetes.c_Bacteroidia.o_Bacteroidales.f_Bacteroidaceae.g_Bacteroides;
k_Bacteria.p_Firmicutes.c_Clostridia.o_Clostridiales.f_Lachnospiraceae.g_Coprococcus;
k_Bacteria.p_Firmicutes.c_Clostridia.o_Clostriales.f._Lachnospiraceae.g_Lachnospira;
k_Bacteria.p_Firmicutes.c_Clostridia.o_Clostridiales.f_Ruminococcaceae.g_Oscillospira;
k_Bacteria.p_Firmicutes.c_Clostridia.o_Clostridiales.f_Tissierellaceae.g_Finegoldia;
k_Bacteria.p_Firmicutes.c_Clostridia.o_Clostridiales.f_Tissierellaceae.g_Peptoniphilus;
k_Bacteria.p_Firmicutes.c_Clostridia.o_Clostridiales.f_Veillonellaceae.g_Dialister;
k_Bacteria.p_Firmicutes.c_Erysipelotrichi.o_Erysipelotrichales.f_Erysipelotrichaceae.g_Eubacterium;
k_Bacteria.p_Proteobacteria.c_Epsilonproteobacteria.o_Campylobacterales.f_Campylobacteraceae.g_Campylobacter; and
k_Bacteria.p_Proteobacteria.c_Gammaproteobacteria.o_Pasteurellales.f_Pasteurellaceae.g_Haemophilus.

In embodiments, the numerical microbiome score is from 0 to 1 and the predetermined reference value for future risk of prostate cancer or future risk of clinically significant prostate cancer is 0.25 or above.

In embodiments, the methods further comprise determining whether a gut microbiome sample from a human is positive for a colibactin-producing E. coli.

In embodiments, determination that the gut microbiome sample from a human is positive for a colibactin-producing E. coli and also at risk of a future prostate cancer, indicates that the subject is at a relatively increased risk of a future prostate cancer compared to a subject at risk of a future prostate cancer but negative for colibactin-producing E. coli.

A method for reducing a risk of a prostate cancer or risk of a future prostate cancer or clinically significant prostate cancer comprising determining, or receiving a determination that, a human subject is at risk of a future prostate cancer by a method described herein, and then administering to the subject one or more agents effective to modify the numerical microbiome score of the gut microbiome of the subject so as to thereby reduce a risk of a future prostate cancer or risk of a future clinically significant prostate cancer in the subject.

In embodiments, wherein the subject is positive for colibactin-producing E. coli, in addition to one or more agents effective to modify the numerical microbiome score of the gut microbiome the subject is administered an anti-colibactin positive E. coli treatment.

In embodiments, the one or more agents effective to modify the numerical microbiome score of the gut microbiome of the subject include a Fecal Microbiota Transplant (FMT); a probiotic supplement; a prebiotic; a phage therapy to selectively eliminate one or more gut bacteria that increase the microbiome score; a Microbiota-Accessible Carbohydrates (MAC) to feed one or more beneficial gut bacteria that decrease the microbiome score; a CRISPR-Cas9 editing agent to modify one or more gut bacteria to decrease the microbiome score; a microbiome banking sample previously taken from the subject when they had a healthy microbiome for future therapeutic use; or a nutritional diet that reduce one or more gut bacteria that increase the microbiome score and/or increase one or more beneficial gut bacteria that decrease the microbiome score.

In embodiments, the anti-colibactin positive E. coli treatment comprises an antibiotic that kills pks-carrying E. coli strain(s), a phage therapy to selectively eliminates pks-carrying E. coli strain(s), a CRISPR-Cas9 editing agent to selectively inhibit colibactin production by pks-carrying E. coli strain(s); a nutritional, FMT, prebiotic or probiotic, that increases non-pks-carrying E. coli strain(s) relative to pks-carrying E. coli strain(s).

In embodiments, the method comprises reducing the numerical microbiome score of the gut microbiome of the subject below 0.25.

In embodiments, the method may be executed based on computer code stored in the one or more memories and executed by the one or more processors. In embodiments, the computer system may be local to a user, remote from the user, or both local and remote (e.g., aspects may involve cloud computing).

In embodiments, the one or more processors may actually perform one or more of the steps of the method and/or cause one or more of the steps of the method to be performed.

In embodiments, the subject, or subjects, is/are human. In preferred embodiments, all data sets and data subsets are obtained from human subjects.

In embodiments, samples have been obtained by a medical professional and not the patient subject. In embodiments, samples have been obtained the patient subject.

In embodiments, the methods can be performed serially. In embodiments, the methods can be performed sequentially on the same subject, or on different subjects.

A predefined condition is a condition or status decided or determined beforehand.

In embodiments, where a numerical range is provided herein, it is understood that all numerical subsets of that range, and all the individual integers contained therein, are provided as embodiments of the invention. For example, 30 to 70 includes the subset of 30 to 35, the subset of 40-60, etc. as well as every individual integer value, e.g. 30, 31, 32, 33, and so on.

“And/or” as used herein, for example with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

One skilled in the art will readily appreciate that the specific methods and results discussed herein are merely illustrative of embodiments of the invention as described more fully in the claims that follow thereafter.

### EXAMPLES

### Study Design and Population

A prospective, observational clinical study design was used collecting fecal samples for gut microbiome analysis and link the microbiome data to the diagnosis of prostate cancer. Two populations were used to obtain fecal samples and prostate cancer status. One group included men presenting for prostate cancer screening as part of our San Antonio Biomarkers of Risk (SABOR) cohort. SABOR is an 18-year prospective 4,174-subject cohort undergoing regular serum prostate-specific antigen (PSA) testing as part of a clinical validation site for the National Cancer Institute's Early Detection Research Network (EDRN). [10] The SABOR cohort represents a usual risk patient population engaged in prostate cancer screening. Men were also enrolled at the point of deciding to proceed to a prostate biopsy, before prophylactic antibiotics are used. The second group of patients are part of a genitourinary repository consisting of men proceeding to a prostate biopsy (IRB #: HSC20050234H), before prophylactic antibiotics are used.

### Outcomes

Our primary outcome was defined as clinically significant prostate cancer defined a grade group 2 (Gleason 3+4) prostate cancer or greater. The primary predictor of prostate cancer is the microbiome score. Our secondary outcome was any prostate cancer to include low-risk (i.e., Grade Group 1) prostate cancer.

### Sample Collection and Preparation

All specimens were collected prospectively, especially before antibiotic use in the biopsy cohort. Both rectal swabs and glove tips were used for fecal microbiome collection. We have studied both methods and found them to be highly correlated with results and DNA yield.[17] The rectal swab technique involved the urology provider by using sterile Medline E-Z Lubricating Jelly and placing the swab approximately 2 inches into the rectum and turning the swab 360 degrees. Once collection was complete, the rectal swab was removed and examined for stool. The physician reinserted the swab if there was no material collected. The physician placed the swab in a 15 mL sterile centrifuge tube containing 1 mL of phosphate-buffered saline (PBS). The swab specimen was stored at 4° C. during transport to the laboratory within 4 hours of collection. Swab was saved and PBS material at −20° C. until and then performed DNA isolation.

### DNA Isolation and Sequencing

For sample input, we attempted to provide a visible amount of stool on the glove tips and swabs. DNA was isolated from fecal samples using our standard operating procedure. [18] A purification was performed of genomic DNA from these respective fecal samples using the QIAamp® Fast DNA Stool Mini Kit according to the kit protocol (Qiagen, Germantown, MD). The DNA concentration was measured using the Thermo Scientific NanoDrop. Genomic DNA was used for amplification of V1-V2 variable region of the 16S rRNA genes with custom-designed primers (F27/R534) and V3-V4 variable region of the 16S rRNA genes following the Illumina 16S metagenomic library preparation guide.[19] Final libraries were quantified, normalized, pooled together, and sequenced by Paired-end sequencing (2×300 bp) using Illumina Miseq platform. The average of 264,727 raw pair reads per sample were generated with read length of 300 bp. The sequences were exported as FASTQ files.

### Statistical Analysis and Bioinformatics

Microbial functional imputation of the microbiome signature: PICRUSt was applied to rarefied taxonomic profiles (15,000 sequences per sample) to infer functional categories associated with taxonomic composition. Computational analyses were performed on V1V2 and V3V4 amplicon regions separately. Alpha diversity calculations were performed using QIIME [20]. Beta-diversity comparative analysis utilized the vegan R package for PERMANOVA calculations. LEfSe was executed via Galaxy server for comparison of groups of interest [21, 22]. Taxa identified as significant across group comparisons were selected for machine learning model training by random forest using the caret R package requiring 10-fold cross-validation. Filtered results using taxa identified by LEfSe were summarized by PICRUSt analysis as KEGG Level 3 categories and analyzed for differential abundance between patient groups of interest using the nonparametric Mann-Whitney test with False Discovery Rate adjustment of P-values.

Statistical analysis Univariant analysis for demographics included chi squared or Fischer's exact test for categorical variables and Student's T test for continuous variable. ANOVA was used for multipole comparisons without pos hoc analysis. Logistic regression was used for multivariant comparison specifically to determine if the microbiome score was significant for predicting the dichotomous outcome of clinically significant cancer when controlling for other factors including PSA. A decision curve analysis will be estimated for the microbiome score values using 5-fold cross validation. Bootstrap 95% confidence intervals were constructed for the differences in net benefit. If the predictive model or biomarker has a 95% CI for the difference exceeds 0 relative to another, then this would be preferred. Using logistic regression, the incremental benefit of the microbiome score values will be evaluated.

### Quality Assurance

### Sequencing

the 16 s V1V2 variable regions were collected and sequenced using. Later in the study sequencing V3V4 regions was also done in addition to V1V2 to calculate the score using both sequencing techniques for correlation.

### Validation

Cross-validation usually performs better than a training and testing set. Therefore, the cohort was divide into a training (2/3) and testing (1/3) to determine performance. The importance scores of each taxon were then compared for similarity between the two model training versions, finding a significant positive correlation (Pearson, r=0.79; P<0.001).

### Change Over Time

Some subjects have had at least one additional collection. The stability of the microbiome signature is compared over time based on these two collections.

### Microbiome Score Performance

The microbiome score was correlated to standard of care risk measurements to include PSA and the Prostate Cancer Prevention Trial risk calculator.

### Decision Analysis Curves

The clinical benefit of the microbiome predictors was examined for csPCa and any grade prostate cancer using decision curve analysis (DCA) implemented by the R package dcurves [23]. The DCA compares a predictor's benefit as a basis for treatment decision relative to treatment for all and treatment for none for a range of probability thresholds. The clinical net benefit is the true positive rate minus the false positive rate scaled by the odds of disease prevalence. Using the validation data set, decision curves were computed for GG2+ and any prostate cancer for microbiome predictors and compared their net benefit relative to PSA.

### Time to Event Analysis

Ability of the microbiome score threshold of 0.25 to group men into high risk or low risk categories was tested. Men diagnosed with prostate cancer within 6 months of the microbiome collection were excluded. The Kaplan-Meier curve was then used for visualization and the log-rank statistical test to determine if there was a significant difference in the incidence of prostate cancer and clinically significant prostate cancer.

### Microbiome Score as a Modifiable Biomarker

Due to the increased risk of prostate cancer in patients with inflammatory bowel disease (IBD), who have aberrant microbiomes, it was sought to first obtain the 16s RNA publicly available data from the Human Microbiome Project 2 (HMP2) subset with patients with IBD. The data between men and women and people with and without IBD will be investigated. Groups will be compared using the Mann-Whitney test and visualize the microbiome score using box and whisker plots. Subsequently, the publicly available data was obtained from a randomized controlled study of fecal transplant for men with IBD. This data will determine if the microbiome score is modifiable. We will calculate the microbiome score of the donors and both intervention and control groups over the period of the study. The microbiome score will be compared before and after intervention using the Mann-Whitney test.

### Results

The results are as set forth in the attached figures and descriptions thereof, and also as additionally described below.

### Characterization of the Study Population

After IRB approvals, a total of 2,028 samples were collected from February 2016 to December 2021. 451 individuals were analyzed from the SABOR screening cohort and 373 individuals from biopsy cohort for a total of 824 (CONSORT diagram). Demographics are shown in Table 1. The groups are broken down into cohorts by prostate biopsy and screening cohorts as well as racial categories.

TABLE 1
Cancer Groups (by grade)

| | GG2+ | GG1 | Negative Biopsy | Low Risk | P |
|---|---|---|---|---|---|
| | (N = 125) | (N = 106) | (N = 143) | (N = 450) | value |
| Age | 68.0 [37.0, 81.0] | 66.0 [47.0, 79.0] | 66.0 [37.0, 81.0] | 68.5 [40.0, 87.0] | <0.001 |
| Body Mass Index | 28.8 [19.3, 46.2] | 29.0 [18.7, 45.9] | 29.1 [18.7, 47.8] | 27.6 [14.6, 71.2] | 0.00166 |
| DRE | | | | | |
| Normal | 64 (51.2%) | 79 (74.5%) | 102 (71.3%) | 404 (89.8%) | <0.001 |
| Abnormal | 57 (45.6%) | 22 (20.8%) | 19 (13.3%) | 34 (7.6%) | |
| Not Performed | 4 (3.2%) | 5 (4.7%) | 22 (15.4%) | 12 (2.7%) | |
| Race | | | | | |
| White | 74 (59.2%) | 54 (50.9%) | 74 (51.7%) | 395 (87.8%) | <0.001 |
| Black | 38 (30.4%) | 35 (33.0%) | 50 (35.0%) | 50 (11.1%) | |
| Other | 13 (10.4%) | 17 (16.0%) | 19 (13.3%) | 5 (1.1%) | |
| Ethnicity | | | | | |
| Non-Hispanic | 89 (71.2%) | 83 (78.3%) | 108 (75.5%) | 309 (68.7%) | <0.001 |
| Hispanic | 34 (27.2%) | 20 (18.9%) | 30 (21.0%) | 141 (31.3%) | |
| Other | 2 (1.6%) | 3 (2.8%) | 5 (3.5%) | 0 (0%) | |
| Family History of PCa | | | | | |
| No | 106 (84.8%) | 83 (78.3%) | 121 (84.6%) | 380 (84.4%) | <0.001 |
| Yes | 3 (2.4%) | 5 (4.7%) | 4 (2.8%) | 69 (15.3%) | |
| Not Reported | 16 (12.8%) | 18 (17.0%) | 18 (12.6%) | 1 (0.2%) | |
| Prostate Specific | 8.06 | 5.89 | 6.33 | 1.40 | <0.001 |
| Antigen Level ng/mL | [0.140, 295] | [0.270, 27.6] | [0.800, 19.3] | [0.100, 54.9] | |
| (PSA) | | | | | |
| Observed Microbial | 62.0 | 63.0 | 63.0 | 63.0 | 0.0582 |
| Species | [29.0, 85.0] | [43.0, 84.0] | [34.0, 94.0] | [38.0, 158] | |
| Shannon Diversity | 3.56 | 3.61 | 3.54 | 3.54 | 0.49 |
| | [1.21, 4.43] | [0.952, 4.39] | [1.12, 4.39] | [1.90, 4.59] | |
| PRIMUS | 0.376 | 0.365 | 0.364 | 0.216 | <0.001 |
| Microbiome Score | [0.07, 0.70] | [0.05, 0.67] | [0.06, 0.66] | [0.02, 0.69] | |

### Quality Control

Amplicon sequenced libraries were submitted for quality trimming, paired-end read merging, chimera removal, host-genome detection removal and other common artifacts associated with 16S rRNA processing (see Methods). Overall, 60 sequenced samples (4.86%) were excluded from consideration due to low coverage after quality control, with statistically similar frequencies of complete subject exclusion between the SABOR and biopsy cohorts (P=0.99; Fisher's Exact Test).

### Microbiome Signature Identification

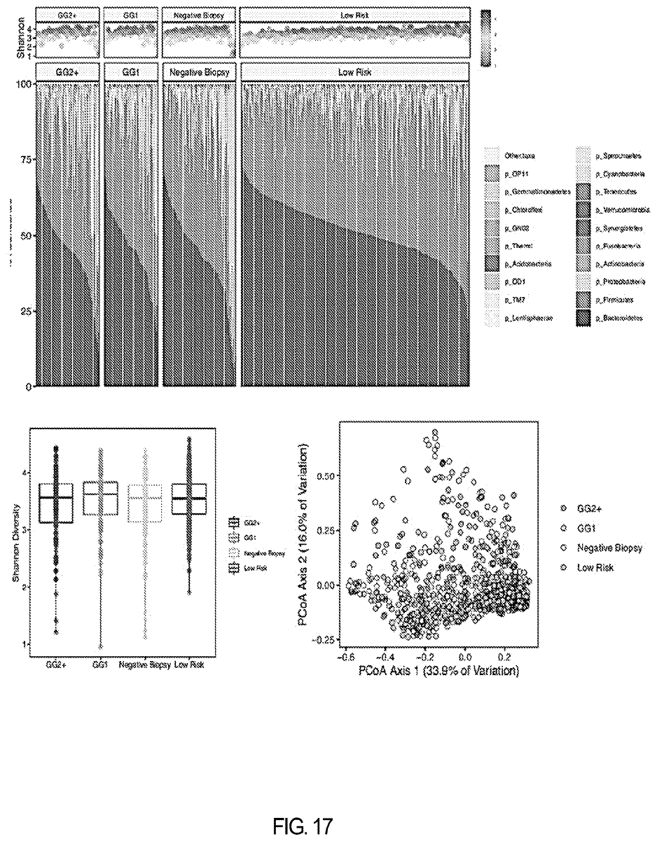

After sequencing, quality check, and metadata hygiene a random forest plot was used to assess the microbiome in various groups to provide a microbiome score (FIG. 17A). The score is a number from 0 to 1 and increasing number is associated with prostate cancer. A cladogram was used to delineate the GG2 microbiome profiles more clearly from GG1 or non-cancer groups. Linear discriminant analysis (LDA) of >2.4 was used as the cut off for valuable microbial profiles. One identified; they were plotted using a circular branching diagram showing the cladistic relationship between several species enriched for GG2 prostate cancer compared to no GG2 prostate cancer. Lastly, an AUC was used and calculated for operational characteristics of the microbiome score. The microbiome score was statically significant whether using either the 10-fold cross validation or training/validation AUC of 69.7 (95% CI 69.4-69.7). The threshold used to call a positive test was a microbiome score of 0.158 that led to a sensitivity, specificity, positive predictive value (PPV) and negative predictive value (NPV) of 75.4%, 57%, 24.17% and 92.72%, respectively (FIG. 17C). The top left shows the top 15 microbes and their importance level in the model. The bottom two histograms show the microbiomes scores of those without cancer or grade group 1 prostate cancer on top (green) compared to the distribution of men with grade group 2 or higher prostate cancer on the bottom (orange).

### Alpha and Beta Diversity

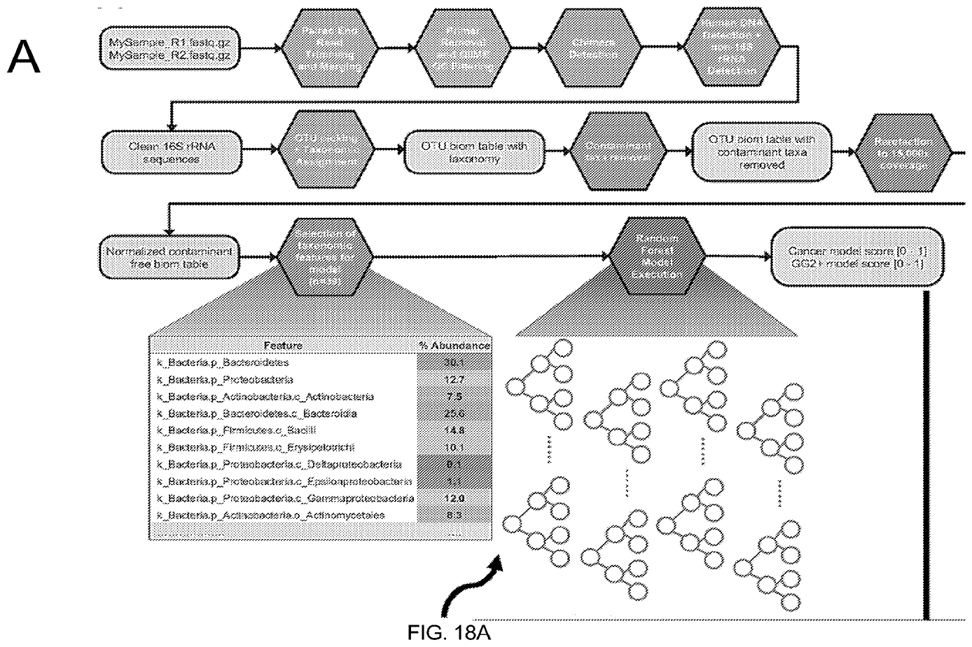

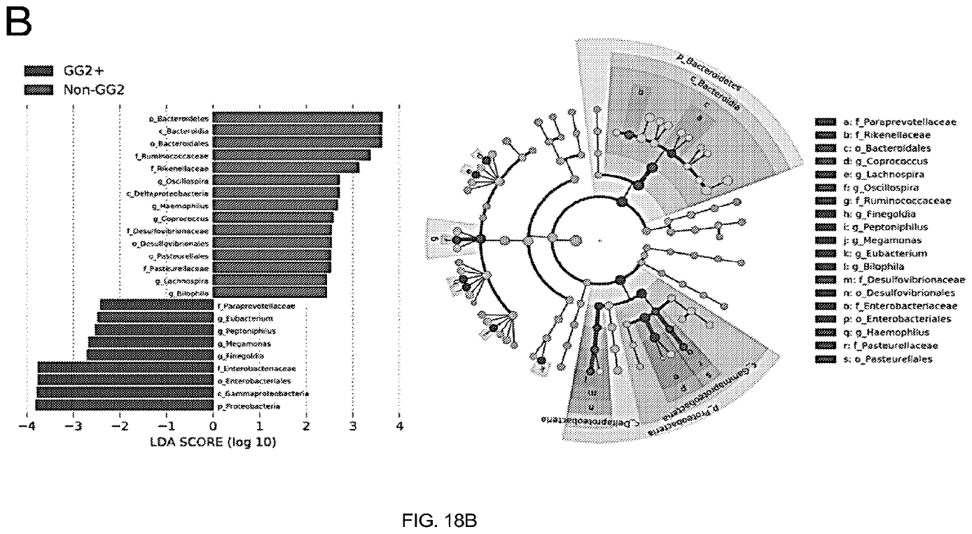

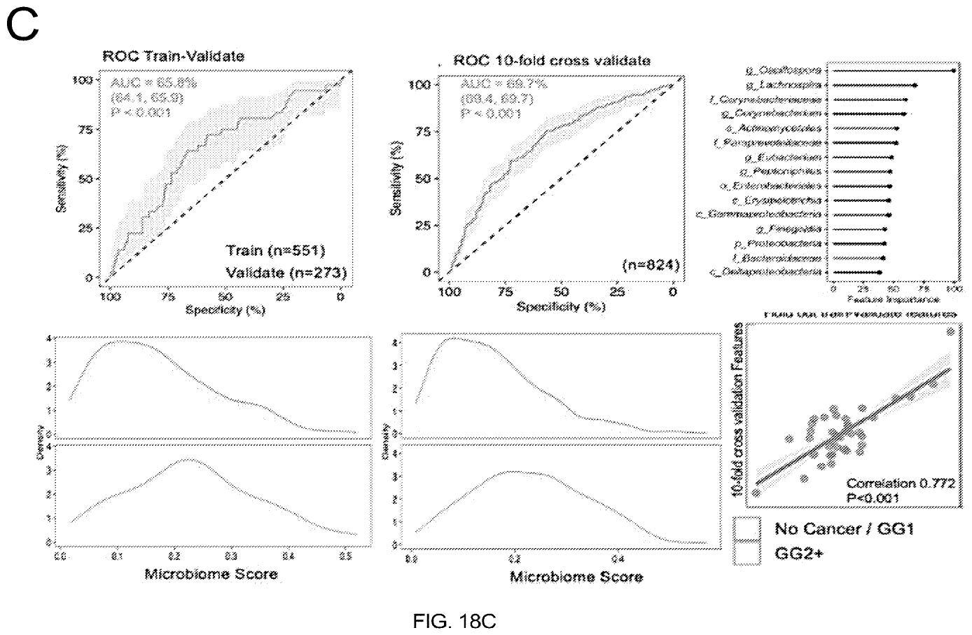

The overall diversity profiles in the four groups corresponding to Table 1 (GG2+, GG1, negative biopsy, and low risk) was displayed. The alpha diversity between groups was not different (FIG. 18A shows the groups and subsequent subgroup analysis of cancer status and race were not statistically different (all p>0.05). However, when comparing beta diversity, differences were noticed between cancer status (Curtis Bray distance, PERMANOVA p<0.01, respectively). Also investigated were populations and race/ethnicity and beta diversity differences noted between groups (both PERMANOVA p<0.01, respectively).

Multivariant Analysis with a Microbiome Signature

There is no difference in number of microbial species across groups when comparing across risk groups. The microbiome score across these groups noted the lowest median score for the low-risk group and the highest median score for the GG2+ group (P<0.001). In Table 2, the results of multivariate analysis with the microbiome score is shown. Controlling for PSA and abnormal prostate exam, the microbiome score was a statistically significant predictor of prostate cancer (p<0.001) controlling for other risk factors.

TABLE 2
Multivariable logistic regression for men that underwent prostate biopsy

| | Clinically Significant | | | Clinically Significant | | | | | |
|---|---|---|---|---|---|---|---|---|---|
| | Prostate Cancer vs. No | | | Prostate Cancer vs. (No | | | Any Grade Prostate | | |
| | Cancer | | | Cancer/Grade 1 Cancer) | | | Cancer vs. No Cancer | | |
| | (n = 125 vs. 593) | | | (n = 125 vs. 699) | | | (n = 231 vs. 593) | | |
| | | 95% | p | | 95% | p | | 95% | p |
| Characteristic | OR¹ | CI¹ | value | OR¹ | CI¹ | value | OR¹ | CI¹ | value |
| Age | 0.98 | 0.95, | 0.2 | 0.99 | 0.96, | 0.5 | 0.98 | 0.96, | 0.1 |
| | | 1.01 | | | 1.02 | | | 1.00 | |
| BMI | 1.05 | 1.00, | 0.043 | 1.04 | 1.00, | 0.044 | 1.04 | 1.00, | 0.051 |
| | | 1.10 | | | 1.09 | | | 1.07 | |
| DRE | | | | | | | | | |
| Normal | — | — | | — | — | | — | — | |
| Abnormal | 5.7 | 3.07, | <0.001 | 4.14 | 2.40, | <0.001 | 3.58 | 2.16, | <0.001 |
| | | 10.7 | | | 7.13 | | | 5.96 | |
| Not Performed | 0.3 | 0.06, | 0.086 | 0.41 | 0.09, | 0.2 | 0.31 | 0.12, | 0.016 |
| | | 1.03 | | | 1.26 | | | 0.77 | |
| Race | | | | | | | | | |
| White | — | — | | — | — | | — | — | |
| Black | 1.84 | 0.95, | 0.069 | 1.52 | 0.84, | 0.2 | 1.81 | 1.11, | 0.018 |
| | | 3.57 | | | 2.75 | | | 2.95 | |
| Other | 2.18 | 0.85, | 0.1 | 1.38 | 0.57, | 0.5 | 2.78 | 1.37, | 0.005 |
| | | 5.41 | | | 3.13 | | | 5.68 | |
| Ethnicity | | | | | | | | | |
| Non-Hispanic | — | — | | — | — | | — | — | |
| Hispanic | 1.4 | 0.74, | 0.3 | 1.39 | 0.77, | 0.3 | 1.09 | 0.67, | 0.7 |
| | | 2.61 | | | 2.48 | | | 1.76 | |
| Other | 1.5 | 0.15, | 0.7 | 1.16 | 0.14, | 0.9 | 1.1 | 0.23, | 0.9 |
| | | 10.2 | | | 6.31 | | | 5.07 | |
| Family History | | | | | | | | | |
| PCa | | | | | | | | | |
| N | — | — | | — | — | | — | — | |
| Y | 0.38 | 0.06, | 0.2 | 0.34 | 0.05, | 0.2 | 0.53 | 0.20, | 0.2 |
| | | 1.37 | | | 1.21 | | | 1.20 | |
| Not Reported | 3.08 | 1.33, | 0.008 | 2.16 | 1.03, | 0.037 | 3.91 | 2.01, | <0.001 |
| | | 7.08 | | | 4.41 | | | 7.80 | |
| PSA | 1.17 | 1.11, | <0.001 | 1.14 | 1.10, | <0.001 | 1.18 | 1.12, | <0.001 |
| | | 1.23 | | | 1.19 | | | 1.23 | |
| Microbiome | 3.5 | 1.87, | <0.001 | 2.87 | 1.58, | <0.001 | 2.79 | 1.80, | <0.001 |
| Score² | | 6.90 | | | 5.51 | | | 4.41 | |

¹OR = Odds Ratio, CI = Confidence Interval,
²based on a threshold of 0.25

We display three different analyses to provide comparisons relevant to our analysis. The left column is csPCa (GG2+) compared to no cancer. These two groups should have the most different demographics including the microbiome. The second group compare csPCa to no csPCA which includes both no cancer and GG1 PCa. Some groups are advocating downgrade GG1 and not consider this a cancer, so we provide that analysis to provide a transparency as well as a larger sample size. The right column compares any grade prostate cancer (GG1 and GG2+) compared to no cancer or a negative biopsy to compare the traditional consideration of the PCa pathologic definition.

### Internal Tests of Robustness

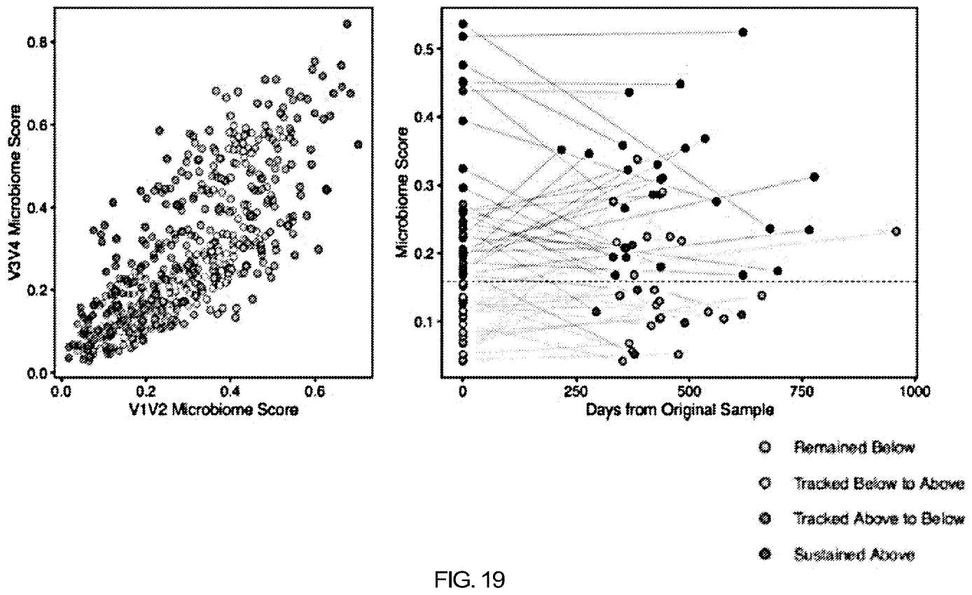

We used two methods to check the robustness of the microbiome score to include comparing the score obtained from sequencing different variable regions and consistency of the microbiome score over time without intervention. We investigate the correlation between the microbiome score generated from variable regions 1 and 2 compared to variable regions 3 and 4 to determine the reproducibility across sequencing techniques. In FIG. 19A, we display a scatter plot with a fit line showing a positive correlation (Pearson correlation=0.77, P<0.001). We also measured the microbiome score for stability of time. Fifty-three subjects had at least two time points to compare. All subjects had V1-V2 variable regions sequenced for consistency. In FIG. 19B, we show the individuals baseline microbiome score and connected them with their future score with a line. We compared the baseline and future microbiome scores noting no statistically significant difference (paired t test, p=0.77). However, 25% (13/53) subjects changed their microbiome score using a threshold 0.158 (GG2+ microbiome score) without intervention (McNemar, p<0.6). We can therefore assume a variation of 25% of score variation above or below the 0.158 threshold over a median of one year.

Microbiome Score Correlated with Clinically Significant Prostate Cancer Risk

The GG2+ microbiome score is correlated with PSA, overall PCPT cancer risk, and PCPT high-risk prostate cancer (r=0.32, 0.29, and 0.32, all p<0.001). The prostate cancer microbiome score indicating risk of any prostate cancer is correlated with PSA, overall PCPT cancer risk, and PCPT high-risk prostate cancer (r=0.40, 0.37, and 0.39, all p<0.001).

### Predicted Functional Analysis

Using Linear discriminant analysis Effect Size (LEfSe) we note caffeine metabolism and glycan metabolism as important aspects of associated with those without GG2 prostate cancer. We found two very strong association with GG2 prostate cancer are similar to those bacteria also associated colon, small cell lung, and bladder cancer.

### Analysis for Secondary Aim of any Prostate Cancer

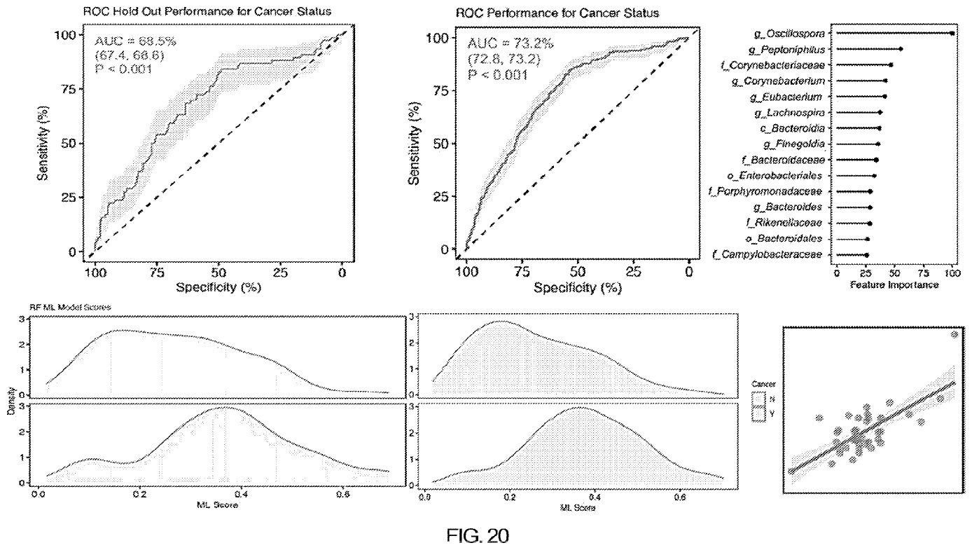

We performed the same analysis on a model where all prostate cancer was compared to no prostate cancer for completeness (Secondary Analysis). The model built for any prostate cancer that includes low risk prostate cancer compared to no cancer. The model required the same base of 39 microbes as the GG2+ model; however, had a different output in machine learning random forest analysis. Using the microbiome score output threshold of 0.256 for the diagnosis of any prostate cancer. Using a training/validation set the microbiome score achieved an AUC of 0.685 (95% CI 0.674-0.686, p<0.001). In a second analysis using a 10-fold cross validation approach the microbiome score attained an AUC of 0.732 (95% CI 0.728-0.732, p<0.001). The operational characteristics of sensitivity, specificity, positive predictive value, and negative predictive value at this threshold are 84.4%, 53.9%, 41.9%, and 89.8%, respectively. A histogram of each microbiome score shown in blue for negative and red for positive for cancer. Because the two scores have different methods of generation, we compared the outputs on a scatter plot with correlation (Pearson 0.78, p<0.001). In univariant analysis, the microbiome score (any prostate cancer vs. no prostate cancer) was statistically significant (p<0.001) and in logistic regression had an odds ratio of 11.7 (95% CI 4.36-33.2, P<0.001).). See FIG. 20.

Microbiome Score Correlated with Clinically Significant Prostate Cancer Risk

The GG2+ microbiome score is correlated with PSA, overall PCPT cancer risk, and PCPT high-risk prostate cancer (r=0.32, 0.29, and 0.32, all p<0.001). The prostate cancer microbiome score indicating risk of any prostate cancer is correlated with PSA, overall PCPT cancer risk, and PCPT high-risk prostate cancer (r=0.40, 0.37, and 0.39, all p<0.001).

### Decision Curve Analysis

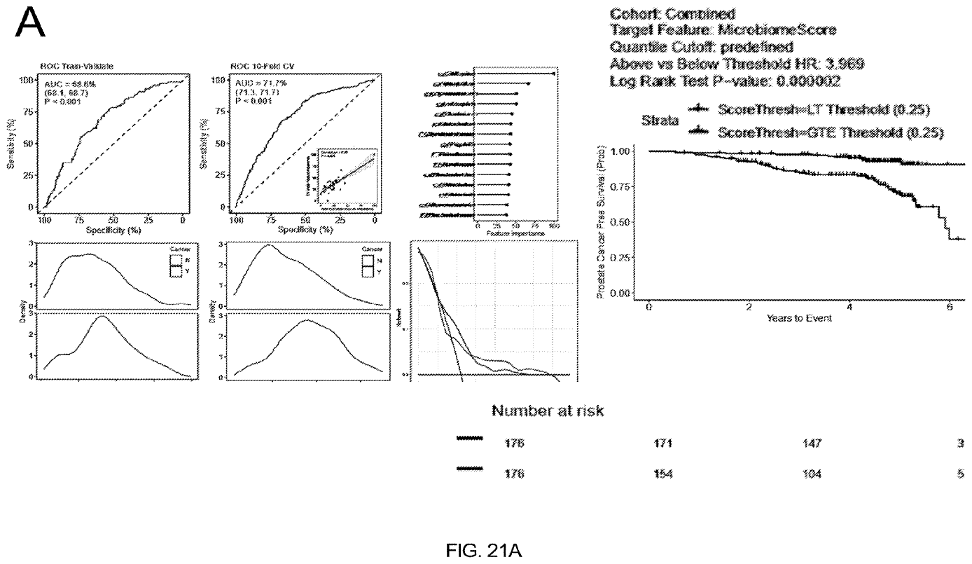

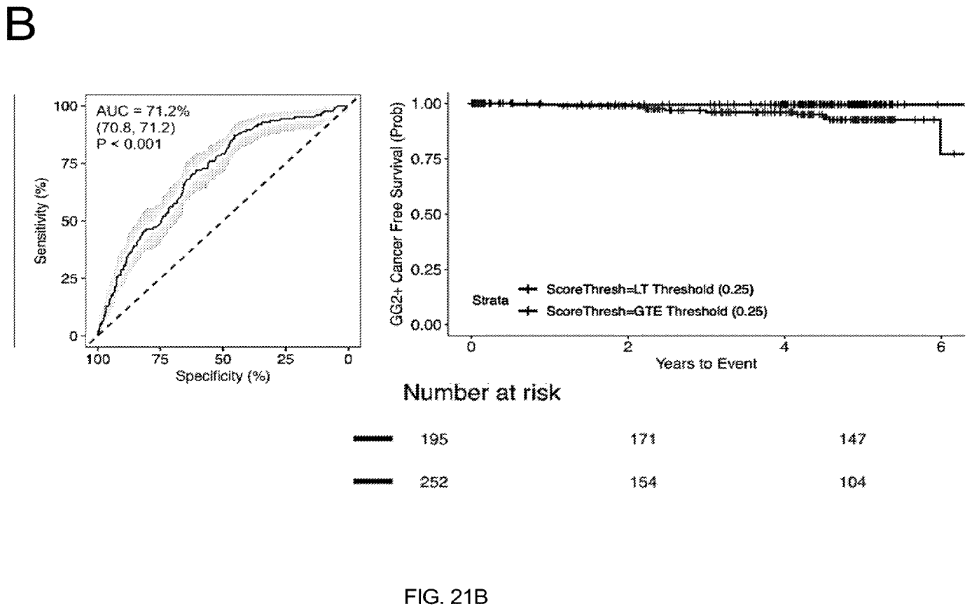

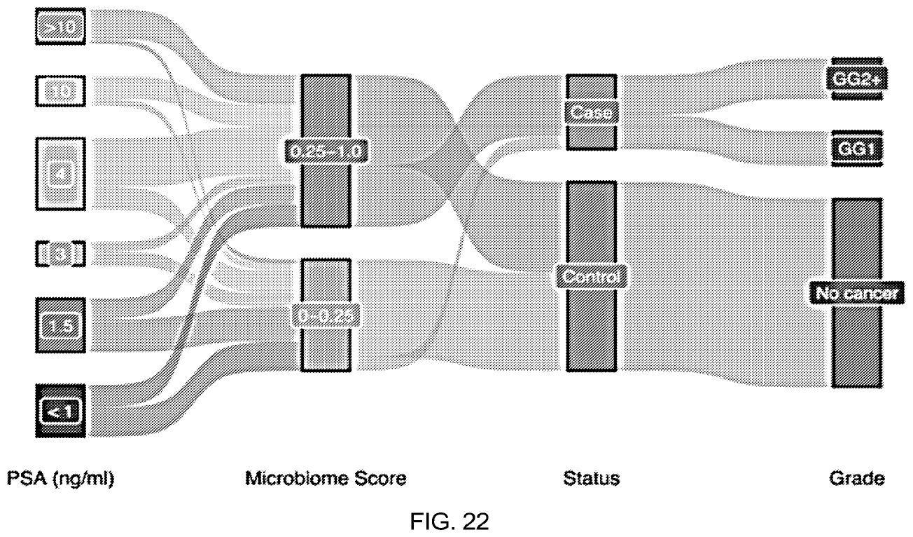

For the decision curve using the microbiome score to predict any prostate cancer, we used thresholds from 10% to 35% which are clinically reasonable thresholds for biopsy (treatment) decisions. In this range, the microbiome score has a greatest benefit relative to PSA, biopsy all or biopsy none. PSA has the greatest net benefit beyond 35% near the intersection of PSA and microbiome score curves, at which point the PSA provides a greater than benefit than the microbiome score. (FIG. 21, iv).

### Time to Event Analysis

We performed two time to event analysis using the same microbiome signature to predict prostate cancer free survival and GG2+ prostate cancer free survival. (i) We display the Kaplan-Meier curves for prostate cancer free survival (PCFS) using the microbiome signature threshold of 0.25 previously discussed that showed a significant reduction in PCFS for those with a high baseline microbiome signature (HR 3.97, 95% CI 2.14-7.36, p<0.001), FIG. 21, v). We also display the Kaplan-Meier curves using the same microbiome signature, but for the endpoint of GG2+ prostate cancer free survival also noting a significant reduction in PCFS in men with a high baseline microbiome signature (HR 11.1, 95% CI 1.42-87.17, P<0.001, FIG. 21, vii). Neither group reached the 50% median endpoints.

### Modification of the Microbiome Score

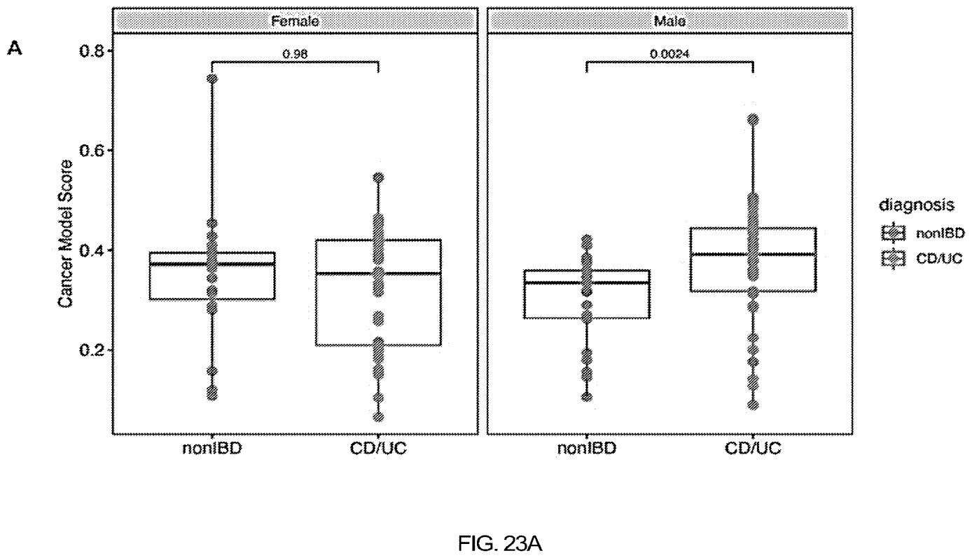

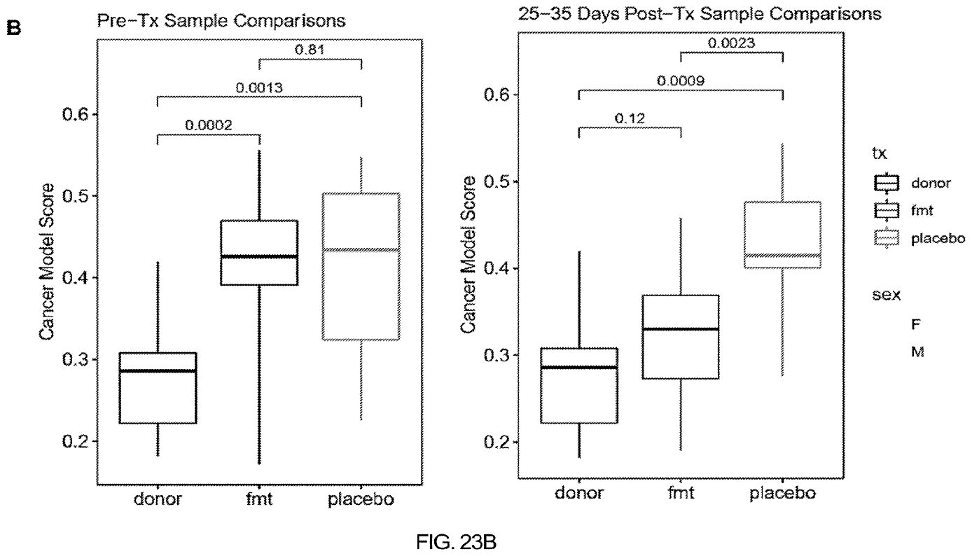

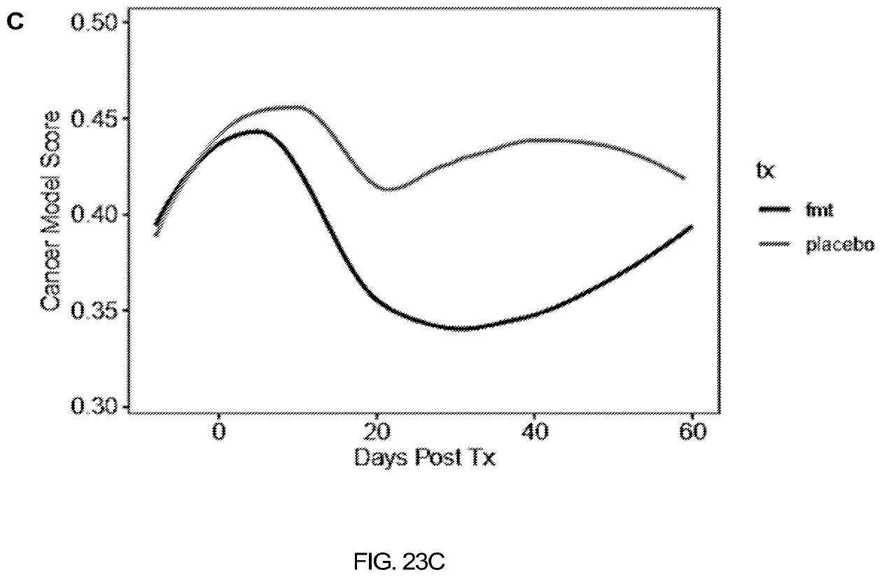

We initially wanted to inspect the microbiomes signature for prostate cancer in patient with inflammatory bowel disease. We obtained the sequencing from the integrative human microbiome project sub study on IBD. [24]. We obtained the publicly available data from the world wide web at hmpdacc.org/ihmp/. They used V4 (MiSeq platform (Illumina) using the 2×250 bp paired end). We analyzed the score comparing IBD vs. non-IBD subjects and divided them by men and women. We showed a significantly higher microbiome signature for prostate cancer in men with IBD compared to controls (p=0.0025), but the scores not different between IBD and non-IBD in women. We then obtained data from Crothers et al. clinical trial using fecal microbiota transplant for patients with inflammatory bowel disease. They used 16s V4 sequencing with 2 donors and 12 participants (6 intervention and 6 control). The two donors were selected for high (top quintile, 11.11 mmol/g and 12.67 mmol/g stool) of butyrate production. We show that the donor stool had a significantly low microbiome score and compared to both groups (international and control) with IBD (both, p<0.001). Then comparing the microbiome signatures after 25-35 days of FMT the intervention group assumed a similar low microbiome score as the donors (p=0.12), while the placebo group was significantly higher than the intervention group (p=0.002). FIGS. 23A-23C.

Colibactin Exposure is Associated with Prostate Cancer Diagnosis

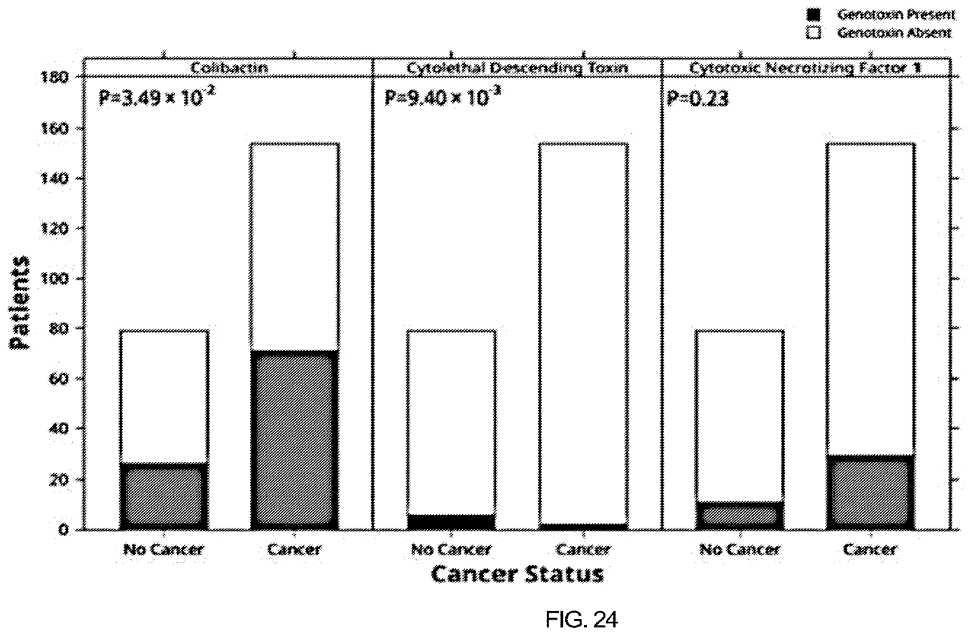

While pks+ E. coli producing colibactin are associated with colorectal tumors, there remains few investigations into their potential role in cancers at other microbiota niches. To assess the link between pks+ E. coli with tumorigenesis in prostate cancer, a retrospective analysis of two observational cohorts of individuals at elevated risk for prostate cancer was conducted. The two clinical cohorts (PSA and San Antonio Center of Biomarkers of Risk for Prostate Cancer [SABOR]) collectively enrolled 388 individuals for whom we have diagnosis results. Table 1 lists the distribution of demographics and subgroups included in the combined cohort and associated clinical measurements. Colibactin exposure was defined as presence of a pks+gene island by PCR. Colibactin exposure was significantly positively associated with prostate cancer diagnosis (OR=1.74, P=0.036, one-sided Fisher's Exact Test). There was no significant positive association between either cytolethal distending toxin or cytotoxic necrotizing factor 1 with prostate cancer diagnosis of any grade (FIG. 24).

TABLE 3
Demographics of the two study population cohorts.

| | Biopsy Cohort | SABOR Cohort | |
|---|---|---|---|
| | (n = 373) | (n = 451) | P value |
| Age | 67.0 [37.0, 81.0] | 69.0 [40.0, 87.0] | <0.001 |
| Body mass index | 29.0 [18.7, 47.8] | 27.6 [14.6, 71.2] | <0.001 |
| Digital rectal exam | | | <0.001 |
| Normal | 244 (65.4%) | 405 (89.8%) | |
| Abnormal | 98 (26.3%) | 34 (7.5%) | |
| Not performed | 31 (8.3%) | 12 (2.7%) | |
| Race | | | <0.001 |
| White | 202 (54.2%) | 395 (87.6%) | |
| Black | 122 (32.7%) | 51 (11.3%) | |
| Other | 49 (13.1%) | 5 (1.1%) | |
| Ethnicity | | | <0.001 |
| Non-Hispanic | 279 (74.8%) | 310 (68.7%) | |
| Hispanic | 84 (22.5%) | 141 (31.3%) | |
| Other | 10 (2.7%) | 0 (0%) | |
| Family History of Prostate | | | <0.001 |
| Cancer | | | |
| No | 309 (82.8%) | 381 (84.5%) | |
| Yes | 12 (3.2%) | 69 (15.3%) | |
| Not Reported | 52 (13.9%) | 1 (0.2%) | |
| Prostate Specific Antigen (PSA) | 6.79 [0.140, 295] | 1.40 [0.100, 54.9] | <0.001 |
| Cancer | | | <0.001 |
| No | 143 (38.3%) | 450 (99.8%) | |
| Yes | 230 (61.7%) | 1 (0.2%) | |
| Grade Group | | | <0.001 |
| GG1 (Gleason 3 + 3) | 105 (28.2%) | 1 (0.2%) | |
| GG2 (Gleason 3 + 4) | 59 (15.8%) | 0 (0%) | |
| GG3 (Gleason 4 + 3) | 26 (7.0%) | 0 (0%) | |
| GG4 (Gleason 4 + 4) | 24 (6.4%) | 0 (0%) | |
| GG5 (Gleason 4 + 5, 5 + 4, or 5 + 5) | 16 (4.3%) | 0 (0%) | |
| Negative | 143 (38.3%) | 450 (99.8%) | |
| Observed microbial species | 63.0 [29.0, 94.0] | 63.0 [38.0, 158] | 0.0115 |
| Shannon diversity | 3.57 [0.952, 4.43] | 3.54 [1.90, 4.59] | 0.625 |
| Microbiome Score | 0.370 [0.050, 0.702] | 0.216 [0.0240, 0.696] | <0.001 |

### Discussion

Our findings have identified a PSA-independent, gut microbiome-based signature associated with clinically significant prostate cancer. We also discovered that a baseline microbiome signature assessment may have increased risk of prostate cancer over the next 4-6 years, which coincided with minimal change on repeat testing up to 1 year apart. Lastly, the microbiome signature is potentially modifiable, opening a new area of clinical investigation to determine if microbiome modification could be a biomarker in prostate cancer prevention clinical trials.

While other studies have identified a few associated bacteria, our study is the first to attribute a predictive microbial signature. Matsushita and colleagues published an 18 microbe score termed the Fecal Microbiome prostate index (n=152) in a Japanese cohort noted AUC of 0.85 for high risk and 0.70 for any cancer.[26] It may be difficult to extrapolate these findings due to the uniqueness of the Japanese microbiome.[27] Our signature reached a similar AUC of 0.71 with good sensitivity (83%) and negative predictive value (89%). While current pre-biopsy biomarkers have better performance metrics, the unique ability of a microbiome signature is the ability to individual lifestyle recommendations. However, we do not know the exact mechanism of the gut microbiome's influence on prostate cancer nor that manipulating the microbiome reduces prostate cancer risk. Using several animal models our working hypothesis is that there is a systemic component that could be related to metabolites or immune alterations or both. [28]

Reichard et al. published that several gut microbe-produced metabolites are associated with lethal prostate cancer. [29] Our group previously published on predicted metabolites associated with prostate cancer. [16] However, new research has investigated microbial interactions with the immune system that may increase the risk of cancer. For instance, the microbiome may interact closely with colonic cells to influence Treg cell differentiation and Tregs have been implicated in prostate cancer formation. [30,31] We used the example of IBD as an extreme example of dysbiosis and the increased risk of prostate cancer. The mechanism of increased cancer in this group has been suspected to be increased inflammation leading to elevated levels of TNF.[32-34]

We acknowledge limitations of our study including the enrollment of two major groups of patients noted differences in demographics. We took this into account in the quality section of the manuscript. We also used different collection techniques between stool, rectal swab, and glove tip techniques, which has previously been published to not significantly effect microbiome analysis [17]. Due to prostate needle traversing the colon to obtain a prostate biopsy in most cases, we did not compare gut microbes to prostate cancer tissue as there may DNA contamination. Other groups have focused on the gut microbiome and treatment outcomes [35]; however, the current study remains the largest and most comprehensive study on gut microbiome, and its association with prostate cancer and prostate cancer risk.

In conclusion, we have developed a PSA independent, gut-microbiome based prostate cancer biomarker. We anticipate such a biomarker may be used to explore the interaction of the gut microbiome with current and future therapeutic strategies as a potential targetable biomarker. Currently there are no prevention strategies for those a genetic or regular risk to follow and reduce their prostate cancer. While this study does not provide causality, our future investigations plan to develop clinical trials to answer these new questions.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly. Those of ordinary skill in the art will recognize that the method and apparatus of the present invention described herein and others implied have many applications; therefore, the present invention which is the subject of this application is not limited by or to the representative examples and/or methods disclosed herein, nor limited by or to the preferred embodiments described herein. Moreover, various other embodiments and modifications to these exemplary embodiments may be made by those skilled in the relevant art without departing from the scope or spirit of these inventions. Accordingly, the inventions are not to be limited by the foregoing specification, except as by the appended claims.

### REFERENCES

1 Siegel, R. L., Miller, K. D., Wagle, N. S. & Jemal, A. Cancer statistics, 2023. CA Cancer J Clin 73, 17-48 (2023). https://doi.org/10.3322/caac.21763
2 Force, U. S. P. S. T. et al. Screening for Prostate Cancer: US Preventive Services Task Force Recommendation Statement. JAMA 319, 1901-1913 (2018). https://doi.org/10.1001/jama.2018.3710
3 Miller, K. D. et al. Cancer treatment and survivorship statistics, 2022. CA Cancer J Clin 72, 409-436 (2022). https://doi.org/10.3322/caac.21731
4 Carlsson, S. V. & Kattan, M. W. Prostate cancer: Personalized risk-stratified screening or abandoning it altogether? Nat Rev Clin Oncol 13, 140-142 (2016). https://doi.org/10.1038/nrclinonc.2016.11
5 Kachuri, L. et al. Genetically adjusted PSA levels for prostate cancer screening. Nat Med 29, 1412-1423 (2023). https://doi.org/10.1038/s41591-023-02277-9
6 McBride, C. M. et al. Understanding the role of cancer worry in creating a “teachable moment” for multiple risk factor reduction. Soc Sci Med 66, 790-800 (2008). https://doi.org/10.1016/j.socscimed.2007.10.014
7 Lippman, S. M. et al. Effect of selenium and vitamin E on risk of prostate cancer and other cancers: the Selenium and Vitamin E Cancer Prevention Trial (SELECT). JAMA 301, 39-51 (2009). https://doi.org/10.1001/jama.2008.864
8 Thompson, I. M. et al. The influence of finasteride on the development of prostate cancer. N Engl J Med 349, 215-224 (2003). https://doi.org/10.1056/NEJMoa030660
9 Ziglioli, F. et al. Impact of modifiable lifestyle risk factors for prostate cancer prevention: a review of the literature. Front Oncol 13, 1203791 (2023). https://doi.org/10.3389/fonc.2023.1203791
10 Qin, J. et al. A human gut microbial gene catalogue established by metagenomic sequencing. Nature 464, 59-65 (2010). https://doi.org/10.1038/nature08821
11 Turnbaugh, P. J. et al. The human microbiome project. Nature 449, 804-810 (2007). https://doi.org/10.1038/nature06244
12 Sepich-Poore, G. D. et al. The microbiome and human cancer. Science 371 (2021). https://doi.org/10.1126/science.abc4552
13 Markowski, M. C. et al. The Microbiome and Genitourinary Cancer: A Collaborative Review. Eur Urol 75, 637-646 (2019). https://doi.org/10.1016/j.eururo.2018.12.043
14 Liss, M. A. et al. Metabolic Biosynthesis Pathways Identified from Fecal Microbiome Associated with Prostate Cancer. Eur Urol 74, 575-582 (2018). https://doi.org/10.1016/j.eururo.2018.06.033
15 Matsushita, M. et al. The gut microbiota associated with high-Gleason prostate cancer. Cancer Sci 112, 3125-3135 (2021). https://doi.org/10.1111/cas.14998
16 Nishijima, S. et al. The gut microbiome of healthy Japanese and its microbial and functional uniqueness. DNA Res 23, 125-133 (2016). https://doi.org/10.1093/dnares/dsw002
17 Kustrimovic, N., Bombelli, R., Baci, D. & Mortara, L. Microbiome and Prostate Cancer: A Novel Target for Prevention and Treatment. Int J Mol Sci 24 (2023). https://doi.org/10.3390/ijms24021511
18 Biragyn, A. & Ferrucci, L. Gut dysbiosis: a potential link between increased cancer risk in ageing and inflammaging. Lancet Oncol 19, e295-e304 (2018). https://doi.org/10.1016/S1470-2045 (18) 30095-0
19 Wheeler, K. M. & Liss, M. A. The Microbiome and Prostate Cancer Risk. Curr Urol Rep 20, 66 (2019). https://doi.org/10.1007/s11934-019-0922-4
20 Liss, M. A. et al. Higher baseline dietary fat and fatty acid intake is associated with increased risk of incident prostate cancer in the SABOR study. Prostate Cancer Prostatic Dis 22, 244-251 (2019). https://doi.org/10.1038/s41391-018-0105-2
21 Liss, M. A. et al. A diagnosis of prostate cancer and pursuit of active surveillance is not followed by weight loss: potential for a teachable moment. Prostate Cancer Prostatic Dis 19, 390-394 (2016). https://doi.org/10.1038/pcan.2016.28
22 Francescone, R., Hou, V. & Grivennikov, S. I. Microbiome, inflammation, and cancer. Cancer J 20, 181-189 (2014). https://doi.org/10.1097/PPO.0000000000000048
23 Porter, C. M., Shrestha, E., Peiffer, L. B. & Sfanos, K. S. The microbiome in prostate inflammation and prostate cancer. Prostate Cancer Prostatic Dis 21, 345-354 (2018). https://doi.org/10.1038/s41391-018-0041-1
24 Thaiss, C. A., Zmora, N., Levy, M. & Elinav, E. The microbiome and innate immunity. Nature 535, 65-74 (2016). https://doi.org/10.1038/nature18847
25 Chen, J., Pitmon, E. & Wang, K. Microbiome, inflammation and colorectal cancer. Semin Immunol 32, 43-53 (2017). https://doi.org/10.1016/j.smim.2017.09.006
26 Ge, Y., Shi, Q., Yao, W., Cheng, Y. & Ma, G. The association between inflammatory bowel disease and prostate cancer risk: a meta-analysis. Prostate Cancer Prostatic Dis 23, 53-58 (2020). https://doi.org/10.1038/s41391-019-0177-7
27 Burns, J. A. et al. Inflammatory Bowel Disease and the Risk of Prostate Cancer. Eur Urol 75, 846-852 (2019). https://doi.org/10.1016/j.eururo.2018.11.039
28 Matsushita, M., Fujita, K. & Nonomura, N. Influence of Diet and Nutrition on Prostate Cancer. Int J Mol Sci 21 (2020). https://doi.org/10.3390/ijms21041447
29 Short, M. I. et al. Comparison of rectal swab, glove tip, and participant-collected stool techniques for gut microbiome sampling. BMC Microbiol 21, 26 (2021). https://doi.org/10.1186/s12866-020-02080-3
30 Besasie, B. D., Shah, D. P., Leach, R. J. & Liss, M. A. Comparison of in Clinic-Based Fecal Microbiome Collection Techniques for Increase in Study Participation and Utilization of Microbiome Analysis. Open Journal of Urology 9, 51 (2019).
31 Youssef, N. et al. Comparison of species richness estimates obtained using nearly complete fragments and simulated pyrosequencing-generated fragments in 16S rRNA gene-based environmental surveys. Appl Environ Microbiol 75, 5227-5236 (2009). https://doi.org/10.1128/aem.00592-09
32 Bolger, A. M., Lohse, M. & Usadel, B. Trimmomatic: a flexible trimmer for
Illumina sequence data. Bioinformatics 30, 2114-2120 (2014). https://doi.org/10.1093/bioinformatics/btu170
33 Caporaso, J. G. et al. QIIME allows analysis of high-throughput community sequencing data. Nat Methods 7, 335-336 (2010). https://doi.org/10.1038/nmeth.f.303
34 Magoc, T. & Salzberg, S. L. FLASH: fast length adjustment of short reads to improve genome assemblies. Bioinformatics 27, 2957-2963 (2011). https://doi.org/10.1093/bioinformatics/btr507
35 Langmead, B. & Salzberg, S. L. Fast gapped-read alignment with Bowtie 2. Nat Methods 9, 357-359 (2012). https://doi.org/10.1038/nmeth. 1923
36 Kuczynski, J. et al. Using QIIME to analyze 16S rRNA gene sequences from microbial communities. Curr Protoc Bioinformatics Chapter 10, Unit 10 17 (2011). https://doi.org/10.1002/0471250953.bi1007s36
37 Oksanen, J. Vegan: community ecology package. http://vegan.r-forge.r-project.org/(2010).
38 Langille, M. G. et al. Predictive functional profiling of microbial communities using 16S rRNA marker gene sequences. Nat Biotechnol 31, 814-821 (2013). https://doi.org/10.1038/nbt.2676
39 Benjamini, Y., Drai, D., Elmer, G., Kafkafi, N. & Golani, I. Controlling the false discovery rate in behavior genetics research. Behav Brain Res 125, 279-284 (2001).
40 Jeng, L. L., Moore, R. M., Jr., Kaczmarek, R. G., Placek, P. J. & Bright, R.
A. How frequently are home pregnancy tests used? Results from the 1988 National Maternal and Infant Health Survey. Birth 18, 11-13 (1991). https://doi.org/10.1111/j.1523-536×.1991.tb00046.x
41 Segata, N. et al. Metagenomic biomarker discovery and explanation. Genome Biol 12, R60 (2011). https://doi.org/10.1186/gb-2011-12-6-r60
42 Kuhn, M. Building Predictive Models in R Using the caret Package. Journal of Statistical Software 28, 1-26 (2008). https://doi.org/10.18637/jss.v028.i05
43 Vickers, A. J., Van Calster, B. & Steyerberg, E. W. Net benefit approaches to the evaluation of prediction models, molecular markers, and diagnostic tests. Bmj 352, i6 (2016). https://doi.org/10.1136/bmj.i6
44 Gelfond, J. A. et al. Prediction of future risk of any and higher-grade prostate cancer based on the PLCO and SELECT trials. BMC Urol 22, 45 (2022). https://doi.org/10.1186/s12894-022-00986-w
45 The Integrative Human Microbiome Project: dynamic analysis of microbiome-host omics profiles during periods of human health and disease. Cell Host Microbe 16, 276-289 (2014). https://doi.org/10.1016/j.chom.2014.08.014
46 Crothers, J. W. et al. Daily, oral FMT for long-term maintenance therapy in ulcerative colitis: results of a single-center, prospective, randomized pilot study. BMC Gastroenterol 21, 281 (2021). https://doi.org/10.1186/s12876-021-01856-9
47 Vickers, A. J. & Elkin, E. B. Decision curve analysis: a novel method for evaluating prediction models. Med Decis Making 26, 565-574 (2006). https://doi.org/10.1177/0272989X06295361
48 Ankerst, D. P. et al. Evaluating the Prostate Cancer Prevention Trial High Grade Prostate Cancer Risk Calculator in 10 international biopsy cohorts: results from the Prostate Biopsy Collaborative Group. World J Urol 32, 185-191 (2014). https://doi.org/10.1007/s00345-012-0869-2
49 Bryant, A. K. et al. Association of Prostate-Specific Antigen Screening Rates With Subsequent Metastatic Prostate Cancer Incidence at US Veterans Health Administration Facilities. JAMA Oncology 8, 1747-1755 (2022). https://doi.org/10.1001/jamaoncol.2022.4319
50 Prevention, C. f. D. C. a. National Health Interview Survey, 2005-2021., <https://progressreport.cancer.gov/detection/prostate_cancer#:~:text=In%202021%2C%203 7.1%25%20of%20men,test%20within%20the%20past%20 year.> (2023).
51 Golombos, D. M. et al. The Role of Gut Microbiome in the Pathogenesis of Prostate Cancer: A Prospective, Pilot Study. Urology 111, 122-128 (2018). https://doi.org/10.1016/j.urology.2017.08.039
52 Bultman, S. J. The microbiome and its potential as a cancer preventive intervention. Semin Oncol 43, 97-106 (2016). https://doi.org/10.1053/j.seminoncol.2015.09.001
53 Liu, Y., Yang, C., Zhang, Z. & Jiang, H. Gut Microbiota Dysbiosis Accelerates Prostate Cancer Progression Through Increased LPCAT1 Expression and Enhanced DNA Repair Pathways. Front Oncol 11, 679712 (2021). https://doi.org/10.3389/fonc.2021.679712
54 Xue, X. et al. The role of the symbiotic microecosystem in cancer: gut microbiota, metabolome, and host immunome. Front Immunol 14, 1235827 (2023). https://doi.org/10.3389/fimmu.2023.1235827
55 Pernigoni, N. et al. Commensal bacteria promote endocrine resistance in prostate cancer through androgen biosynthesis. Science 374, 216-224 (2021). https://doi.org/10.1126/science.abf8403

1. A method of determining whether a gut microbiome sample from a human subject is associated with a clinically significant cancer comprising:
(a) obtaining or having obtained a gut microbiome-containing sample from a human subject;
(b) performing a genetic analysis on the gut microbiome-containing sample so as to determine a (i) presence or not and (ii) abundance value of each of the following taxa:
g_Oscillospora;
g_Lachnospira;
f_Corynebacteriaceae;
g_Corynebacterium;
o_Actinomycetales;
f Paraprevotellaceae;
g_Eubacterium;
g_Peptoniphilus;
o_Enterobacteriales;
c_Erysipelotrichi;
c_Gammaproteobacteria;
g_Finegoldia;
p_Proteobacteria,
f Bacteroidaceae; and
c_Deltaproteobacteria;
(c) generating a numerical microbiome score from the genetic analysis performed in (b);
(d) comparing the numerical microbiome score generated in (c) with a predetermined reference value which delineates a cutoff between (i) clinically significant cancer and (ii) low-risk cancer/negative biopsy, so as to determine if the gut microbiome sample is associated with a clinically significant cancer,
wherein a numerical microbiome score equal to or greater than the predetermined reference value indicates the sample comes from a human subject at risk of clinically significant cancer and a numerical microbiome score less than the predetermined reference value indicates the sample comes from a human subject with low-risk of cancer/negative biopsy.

2. A method of detecting the presence or absence of a clinically significant cancer in a human subject, the method comprising:
(a) obtaining or having obtained a gut microbiome-containing sample from a human subject;
(b) performing a genetic analysis on the gut microbiome-containing sample, so as to determine a (i) presence or not and (ii) abundance value of each of the following taxa:
g_Oscillospora;
g_Lachnospira;
f_Corynebacteriaceae;
g_Corynebacterium;
o_Actinomycetales;
f Paraprevotellaceae;
g_Eubacterium;
g_Peptoniphilus;
o_Enterobacteriales;
c_Erysipelotrichi,
c_Gammaproteobacteria;
g_Finegoldia,
p_Proteobacteria,
f Bacteroidaceae; and
c_Deltaproteobacteria;
(c) generating a numerical microbiome score from the genetic analysis performed in (b);
(d) detecting:
(i) the presence of a clinically significant cancer if the numerical microbiome score generated in (c) is equal to or greater than a predetermined reference value which delineates a cutoff between clinically significant cancer and low-risk cancer/negative biopsy; and
(ii) the absence of a clinically significant cancer, defined as low-risk of cancer/negative biopsy, if the numerical microbiome score generated in (c) is less than the predetermined reference value.

3. (canceled)

4. (canceled)

5. The method of claim 1, wherein the numerical microbiome score generated in (c) is generated by:
(Y) for each bacterial taxon listed in (b):
tabulating the bacterial taxon abundance value according to its read count and converting to a percentage abundance value; and
providing the percentage abundance value to a random forest model, wherein each tree in the random forest model applies its own threshold(s) to produce a classification output;
and
(Z) summing all classification outputs produced in (Y) from all the bacterial taxa so as to create a numerical microbiome score reflecting the majority vote for the sample.

6. The method of claim 5, wherein the percentage abundance values are provided to a random forest model associated with determining a risk of a clinically significant cancer.

7. The method of claim 5, wherein the percentage abundance values are provided to a random forest associated with determining a risk of a cancer.

8. The method of claim 1, wherein the method further comprises determining whether a gut microbiome sample from a human subject is associated with a risk of a cancer and wherein a second numerical microbiome score is generated by:
(W) for each bacterial taxon listed in (b):
tabulating the bacterial taxon abundance value according to its read count and converting to a percentage abundance value; and
providing the percentage abundance value to a random model associated with determining a risk of cancer, wherein each tree in the random forest model applies its own threshold(s) to produce a classification output;
and
(X) summing all classification outputs produced in (W) from all the bacterial taxa so as to create a second numerical microbiome score reflecting the majority vote for the sample,
wherein a second numerical microbiome score equal to or greater than a second predetermined reference value which delineates a cutoff between (i) a risk of a cancer and (ii) no current risk of cancer indicates the sample comes from a human subject at risk of a cancer and a second numerical microbiome score less than the second predetermined reference value indicates the sample comes from a human subject not currently at risk of cancer.

9. The method of claim 1, wherein the method further comprises whether a gut microbiome sample from a human subject is associated with a risk of a clinically significant cancer and wherein a second numerical microbiome score is generated by:
(U) for each bacterial taxon listed in (b):
tabulating the bacterial taxon abundance according to its read count and converting to a percentage abundance value; and
providing the percentage abundance value to a random model associated with determining a clinically significant cancer, wherein each tree in the random forest model applies its own threshold(s) to produce a classification output;
and
(V) summing all tree classification outputs produced in (U) from all the bacterial taxa so as to create a second numerical microbiome score reflecting the majority vote for the sample,
wherein a second numerical microbiome score equal to or greater than a second predetermined reference value which delineates a cutoff between (i) a clinically significant cancer and (ii) low-risk cancer/negative biopsy indicates the sample comes from a human subject at risk of a clinically significant cancer and a numerical microbiome score less than the second predetermined reference value indicates the sample comes from a human subject of low-risk cancer/negative biopsy.

10. The method of claim 1, wherein the following taxa are the subject of the genetic analysis:
k_Bacteria.p_Proteobacteria
k_Bacteria.p_Actinobacteria.c_Actinobacteria
k_Bacteria.p_Bacteroidetes.c_Bacteroidia
k_Bacteria.p_Firmicutes.c_Bacilli
k_Bacteria.p_Firmicutes.c_Erysipelotrichi
k_Bacteria.p_Proteobacteria.c_Deltaproteobacteria
k_Bacteria.p_Proteobacteria.c_Epsilonproteobacteria
k_Bacteria.p_Proteobacteria.c_Gammaproteobacteria
k_Bacteria.p_Actinobacteria.c_Actinobacteria.o_Actinomycetales
k_Bacteria.p_Bacteroidetes.c_Bacteroidia.o_Bacteroidales
k_Bacteria.p_Firmicutes.c_Bacilli.o_Lactobacillus
k_Bacteria.p_Firmicutes.c_Erysipelotrichi.o_Erysipelotrichales
k_Bacteria.p_Proteobacteria.c_Deltaproteobacteria.o_Desulfovibrionales
k_Bacteria.p_Proteobacteria.c_Epsilonproteobacteria.o_Campylobacterales
k_Bacteria.p_Proteobacteria.c_Gammaproteobacteria.o_Enterobacteriales
k_Bacteria.p_Actinobacteria.c_Actinobacteria.o_Actinomycetales.f_Corynebacteriaceae
k_Bacteria.p_Bacteroidetes.c_Bacteroidia.o_Bacteroidales.f_Bacteroidaceae
k_Bacteria.p_Bacteroidetes.c_Bacteroidia.o_Bacteroidales.f_Odoribacteraceae
k_Bacteria.p_Bacteroidetes.c_Bacteroidia.o_Bacteroidales.f_Paraprevotellaceae
k_Bacteria.p_Bacteroidetes.c_Bacteroidia.o_Bacteroidales.f_Porphyromonadaceae
k_Bacteria.p_Bacteroidetes.c_Bacteroidia.o_Bacteroidales.f_Rikenellaceae
k_Bacteria.p_Firmicutes.c_Clostridia.o_Clostridiales.f_Ruminococcaceae
k_Bacteria.p_Firmicutes.c_Clostridia.o_Clostridiales.f_Tissierellaceae
k_Bacteria.p_Firmicutes.c_Erysipelotrichi.o_Erysipelotrichales.f Erysipelotrichaceae
k_Bacteria.p_Proteobacteria.c_Deltaproteobacteria.o_Desulfovibrionales.f Desulfovibrionaceae
k_Bacteria.p_Proteobacteria.c_Epsilonproteobacteria.o_Campylobacterales.f_Campylobacteraceae
k_Bacteria.p_Proteobacteria.c_Gammaproteobacteria.o_Enterobacteriales.f_Enterobacteriaceae
k_Bacteria.p_Actinobacteria.c_Actinobacteria.o_Actinomycetales.f_Corynebacteriaceae.g_Corynebacterium
k_Bacteria.p_Bacteroidetes.c_Bacteroidia.o_Bacteroidales.f_Bacteroidaceae.g_Bacteroides
k_Bacteria.p_Firmicutes.c_Clostridia.o_Clostridiales.f_Lachnospiraceae.g_Coprococcus
k_Bacteria.p_Firmicutes.c_Clostridia.o_Clostridiales.f_Lachnospiraceae.g_Lachnospira k_Bacteria.p_Firmicutes.c_Clostridia.o_Clostridiales.f_Ruminococcaceae.g_Oscillospira
k_Bacteria.p_Firmicutes.c_Clostridia.o_Clostridiales.f_Tissierellaceae.g Finegoldia
k_Bacteria.p_Firmicutes.c_Clostridia.o_Clostridiales.f_Tissierellaceae.g_Peptoniphilus
k_Bacteria.p_Firmicutes.c_Clostridia.o_Clostridiales.f_Veillonellaceae.g Dialister
k_Bacteria.p_Firmicutes.c_Erysipelotrichi.o_Erysipelotrichales.f_Erysipelotrichaceae.g_Eubacterium
k_Bacteria.p_Proteobacteria.c_Epsilonproteobacteria.o_Campylobacterales.f_Campylobacteraceae.g_Campylobacter,
k_Bacteria.p_Proteobacteria.c_Gammaproteobacteria.o_Pasteurellales.f_Pasteurellaceae.g_Haemophilus).

11. The method of claim 1, wherein the cancer is prostate cancer.

12. The method of claim 1, wherein the clinically significant cancer is Grade Group 2 (GG2) or greater and the low-risk cancer/negative biopsy is Grade Group 1 (GG1) or below.

13. The method of claim 1, wherein the cancer is colon cancer or lung cancer.

14. The method of claim 1, wherein the sample is a fecal sample.

15. (canceled)

16. The method of claim 1, wherein the sample has been obtained by the subject.

17. The method of claim 1, wherein the genetic analysis is performed for a variable region of bacterial 16S rRNA genes.

18. The method of claim 1, wherein the genetic analysis comprises one or more of paired end read trimming and merging; primer removal; QIIME; QC filtering; chimera detection; human DNA detection; and non-16S rRNA gene detection, so as to produce a set of 16S rRNA gene sequences for analysis.

19. The method of claim 1, further comprising filtering out from the analysis genetic analysis, presence indication, and/or abundance values of the following background taxa prior to calculating the numerical microbiome score:
c_Chloroplast
c Alphaproteobacteria
f_Propionibacteriaceae
o_Oceanospirillales
f_Intrasporangiaceae
f Micrococcaceae
f_Cytophagaceae
o_Hydrogenophilales
f_Alcaligenaceae
f_Burkholderiaceae
f_Burkholderiales_incertae_sedis
f_Comamonadaceae
f_Oxalobacteraceae.

20. The method of claim 1, wherein the genetic analysis is performed on variable regions 1 and 2 and/or variable regions 3 and 4 of the 16S rRNA gene of each taxon.

21. The method of claim 1, wherein the numerical microbiome score generated in (c) is generated by, for each bacterial taxon listed in (b):
(i) ascribing a 1 or a 0 if present or not present in the sample, respectively, so as to generate a presence value;
(ii) ascribing a predetermined weighting value to each bacterial taxon, so as to generate a weighted bacteria taxon value;
(iii) modifying the abundance value determined by a predetermined weighting for each bacterial taxon, so as to generate a weighted abundance value; and
(iv) inputting the presence value, weighted abundance value, and weighted bacterial taxon value into a formula and applying the formula so as to generate an output which is the numerical microbiome score.

22. (canceled)

23. (canceled)

24. (canceled)

25. (canceled)

26. (canceled)

27. (canceled)

28. The method of claim 26, wherein the numerical microbiome score generated in b) is generated by:
(Y) for each bacterial taxon listed in a):
tabulating the bacterial taxon abundance value according to its read count and converting to a percentage abundance value; and
providing the percentage abundance value to a random forest model, wherein each tree in the random forest model applies its own threshold(s) to produce a classification output;
and
(Z) summing all classification outputs produced in (Y) from all the bacterial taxa so as to create a numerical microbiome score reflecting the majority vote for the sample.

29. The method of claim 28, wherein the percentage abundance values are provided to a random forest model associated with determining a clinically significant cancer.

30-82. (canceled)
